(19) United States
(12) Patent Application Publication
(10) Pub. No.: US 2022/0363758 A1
(43) Pub. Date: Nov. 17, 2022

(54) Heterodimeric Antibodies That Bind to CD38 and CD3

(71) Applicant: Sorrento Therapeutics, Inc., San Diego, CA (US)

(72) Inventors: Xiao He, Lincolnshire, IL (US); Yanliang Zhang, San Diego, CA (US); Yun Wei Lai, San Diego, CA (US); Gunnar F. Kaufmann, San Diego, CA (US); Barbara A. Swanson, Encinitas, CA (US); Lisa Diane Kerwin, San Diego, CA (US); Susan M. Richards, San Diego, CA (US)

(73) Assignee: Sorrento Therapeutics, Inc., San Diego, CA (US)

(21) Appl. No.: 17/622,087

(22) PCT Filed: Jun. 30, 2020
(86) PCT No.: PCT/US2020/040360

§ 371 Date: Dec. 22, 2021

Related U.S. Application Data
(60) Provisional application No. 62/869,343, filed on Jul. 1, 2019.
(60) Provisional application No. 62/890,163, filed on Aug. 22, 2019.
(60) Provisional application No. 62/945,350, filed on Dec. 9, 2019.

(51) Int. Cl. C07K 16/28; A61P 35/02

(52) U.S. Cl. CPC C07K 16/2809; C07K 16/2896; A61P 35/02; A61K 2039/505

(57) ABSTRACT

The present disclosure provides heterodimeric antibodies that bind to two different target antigens at the same time. In one embodiment, the heterodimeric antibodies are bispecific antibodies. In one embodiment, the heterodimeric antibodies comprise three polypeptides including: a first polypeptide comprising an scFv-Fc fusion polypeptide; a second polypeptide comprising an immunoglobulin heavy chain; and a third polypeptide comprising an immunoglobulin light chain. In one embodiment, the first polypeptide includes one or more point mutations that confer increased thermal-stability to the first polypeptide.

### CROSS-REFERENCE TO RELATED APPLICATIONS

This application claims the benefit of priority under 35 U.S.C. § 119 to U.S. provisional application No. 62/869,343, filed Jul. 1, 2019, U.S. provisional application No. 62/890,163, filed Aug. 22, 2019, and U.S. provisional application No. 62/945,350, filed Dec. 9, 2019. The disclosures of all of the aforementioned applications are incorporated by reference in their entireties.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### TECHNICAL FIELD

The present disclosure provides heterodimeric antibodies that bind to two different target antigens at the same time (e.g., simultaneously). The present disclosure provides heterodimeric antibodies, nucleic acids encoding the heterodimeric antibodies, host cells expressing the heterodimeric antibodies, and methods of use.

### INTRODUCTION

Monoclonal antibody-based therapeutics have been used successfully to treat cancer and autoimmune/inflammatory disorders. The use of combination therapies, involving administration of two different monoclonal antibodies is also an accepted therapeutic method for treating diseases. Combination therapy may also employ bispecific antibodies, where the bispecific antibody is single immunoglobulin molecule designed to co-engage two different antigens. Bispecific antibodies that are designed to generate immune cell synapse can bind antigens on effector cells and tumor-associated antigens expressed by tumors which leads to tumor-selective cytotoxic cell killing. The present disclosure provides heterodimeric antibodies that bind to two different target antigens at the same time. In one embodiment, the heterodimeric antibodies are bispecific antibodies.

### SUMMARY

The present disclosure provides heterodimeric antibodies that bind to two different target antigens at the same time (e.g., simultaneously).

The present disclosure provides a heterodimeric antibody that binds a tumor-associated antigen and a T cell receptor, the antibody comprising: (a) a first polypeptide comprising an scFv and a first Fc region, wherein the scFv forms a first antigen binding domain that binds the T cell receptor; (b) a second polypeptide comprising a heavy chain variable region and a second Fc region; and (c) a third polypeptide comprising a light chain variable region, wherein the heavy chain variable region of the second polypeptide and the light chain variable region of the third polypeptide form a second antigen binding domain that binds the tumor-associated antigen.

The present disclosure provides a heterodimeric antibody that binds a tumor-associated antigen and a T cell receptor, the antibody comprising: (a) a first polypeptide comprising an scFv and a first Fc region, wherein the scFv includes a first heavy chain variable region (VHa) and a first light chain variable region (VLa) joined together by a peptide linker, and wherein the first heavy chain variable region (VHa) and a first light chain variable region (VLa) form a first antigen binding domain that binds the T cell receptor; (b) a second polypeptide comprising a second heavy chain variable region (VHb) and a second Fc region; and (c) a third polypeptide comprising a second light chain variable region (VLb), wherein the second heavy chain variable region (VHb) of the second polypeptide and the second light chain variable region (VLb) of the third polypeptide form a second antigen binding domain that binds the tumor-associated antigen.

The present disclosure provides a heterodimeric antibody that binds a tumor-associated antigen and a T cell receptor, the antibody comprising: (a) a first polypeptide comprising an scFv and a first Fc region, wherein the scFv includes a first heavy chain variable region (VHa) and a first light chain variable region (VLa) joined together by a peptide linker, wherein the scFv is joined to the first Fc region by a first hinge region, and wherein the first heavy chain variable region (VHa) and a first light chain variable region (VLa) form a first antigen binding domain that binds the T cell receptor; (b) a second polypeptide comprising a second heavy chain variable region (VHb) and a second Fc region, wherein the second heavy chain variable region (VHb) is joined to the second Fc region by a second hinge region, and wherein the first and second hinge regions form a disulfide bond; and (c) a third polypeptide comprising a second light chain variable region (VLb), wherein the second heavy chain variable region (VHb) of the second polypeptide and the second light chain variable region (VLb) of the third polypeptide form a second antigen binding domain that binds the tumor-associated antigen.

In one embodiment, the heterodimeric antibody binds a tumor-associated antigen which comprises a CD38 antigen.

In one embodiment, the heterodimeric antibody binds a T cell receptor which comprises a CD3 antigen.

In one embodiment, the first heavy chain variable region (VHa) and the first light chain variable region (VLa) of the first polypeptide comprise fully human immunoglobulin sequences.

In one embodiment, the second heavy chain variable region (VHb) of the second polypeptide comprise fully human immunoglobulin sequences, and the second light chain variable region (VLb) of the third polypeptide comprise fully human immunoglobulin sequences.

In one embodiment, the first heavy chain variable region (VHa) of the first polypeptide comprises an amino acid sequence that is at least 95% identical to one of the sequences selected from SEQ ID NOS:22, 28, 30, 32, 34, 36 and 38.

In one embodiment, the first light chain variable region (VLa) of the first polypeptide comprises an amino acid sequence that is at least 95% identical to one of the sequences selected from SEQ ID NOS:24, 29, 31, 33, 35, 37 and 39.

In one embodiment, the first heavy chain variable region (VHa) of the first polypeptide comprises the CDR-H1, CDR-H2, and CDR-H3 sequences of a heavy chain variable region sequence selected from SEQ ID NOS:22, 28, 30, 32, 34, 36 and 38.

In one embodiment, the first light chain variable region (VLa) of the first polypeptide comprises the CDR-L1, CDR-L2, and CDR-L3 sequences of a light chain variable region sequence selected from SEQ ID NOS:24, 29, 31, 33, 35, 37 and 39. In one embodiment, the first heavy chain variable region (VHa) of the first polypeptide comprises the CDR-H1, CDR-H2, and CDR-H3 sequences of a heavy chain variable region sequence selected from SEQ ID NOS:22, 28, 30, 32, 34, 36 and 38 and the first light chain variable region (VLa) of the first polypeptide comprises the CDR-L1, CDR-L2, and CDR-L3 sequences of a light chain variable region sequence selected from SEQ ID NOS:24, 29, 31, 33, 35, 37 and 39.

In one embodiment, the second heavy chain variable region (VHb) of the second polypeptide comprises the CDR-H1, CDR-H2, and CDR-H3 sequences of a heavy chain variable region sequence selected from SEQ ID NOS:1, 6, 8, 10, 12, 14, 40, 42, 44, 46, 48, 50, 52, 54, 56, 58, 60, 62 and 64.

In one embodiment, the second light chain variable region (VLb) of the third polypeptide comprises the CDR-L1, CDR-L2, and CDR-L3 sequences of a light chain variable region sequence selected from SEQ ID NOS:16, 18, 20, 41, 43, 45, 47, 49, 51, 53, 55, 57, 59, 61, 63 and 65. In one embodiment, the second heavy chain variable region (VHb) of the second polypeptide comprises the CDR-H1, CDR-H2, and CDR-H3 sequences of a heavy chain variable region sequence selected from SEQ ID NOS:1, 6, 8, 10, 12, 14, 40, 42, 44, 46, 48, 50, 52, 54, 56, 58, 60, 62 and 64 and the second light chain variable region (VLb) of the third polypeptide comprises the CDR-L1, CDR-L2, and CDR-L3 sequences of a light chain variable region sequence selected from SEQ ID NOS:16, 18, 20, 41, 43, 45, 47, 49, 51, 53, 55, 57, 59, 61, 63 and 65.

In one embodiment, the first heavy chain variable region (VHa) of the first polypeptide comprises the CDR-H1, CDR-H2, and CDR-H3 sequences of a heavy chain variable region sequence selected from SEQ ID NOS:22, 28, 30, 32, 34, 36 and 38 and the first light chain variable region (VLa) of the first polypeptide comprises the CDR-L1, CDR-L2, and CDR-L3 sequences of a light chain variable region sequence selected from SEQ ID NOS:24, 29, 31, 33, 35, 37 and 39; and the second heavy chain variable region (VHb) of the second polypeptide comprises the CDR-H1, CDR-H2, and CDR-H3 sequences of a heavy chain variable region sequence selected from SEQ ID NOS:1, 6, 8, 10, 12, 14, 40, 42, 44, 46, 48, 50, 52, 54, 56, 58, 60, 62 and 64 and the second light chain variable region (VLb) of the third polypeptide comprises the CDR-L1, CDR-L2, and CDR-L3 sequences of a light chain variable region sequence selected from SEQ ID NOS:16, 18, 20, 41, 43, 45, 47, 49, 51, 53, 55, 57, 59, 61, 63 and 65.

In one embodiment, the second heavy chain variable region (VHb) of the second polypeptide comprises an amino acid sequence that is at least 95% identical to one of the sequences selected from SEQ ID NOS:1, 6, 8, 10, 12, 14, 19, 40, 42, 44, 46, 48, 50, 52, 54, 56, 58, 60, 62, 64 and 101.

In one embodiment, the second light chain variable region (VLb) of the third polypeptide comprises an amino acid sequence that is at least 95% identical to one of the sequences selected from SEQ ID NOS:16, 18, 20, 41, 43, 45, 47, 49, 51, 53, 55, 57, 59, 61, 63 and 65. In one embodiment, the second heavy chain variable region (VHb) of the second polypeptide comprises an amino acid sequence that is at least 95% identical to one of the sequences selected from SEQ ID NOS:1, 6, 8, 10, 12, 14, 19, 40, 42, 44, 46, 48, 50, 52, 54, 56, 58, 60, 62, 64 and 101 and the second light chain variable region (VLb) of the third polypeptide comprises an amino acid sequence that is at least 95% identical to one of the sequences selected from SEQ ID NOS:16, 18, 20, 41, 43, 45, 47, 49, 51, 53, 55, 57, 59, 61, 63 and 65.

In one embodiment, the first heavy chain variable region (VHa) of the first polypeptide comprises the amino acid sequence of any one of SEQ ID NOS:22, 28, 30, 32, 34, 36 and 38 and/or the first light chain variable region (VLa) of the first polypeptide comprises the amino acid sequence of any one of SEQ ID NOS:24, 29, 31, 33, 35, 37 and 39.

In one embodiment, the second heavy chain variable region (VHb) of the second polypeptide comprises the amino acid sequence of any one of SEQ ID NOS:1, 6, 8, 10, 12, 14, 40, 42, 44, 46, 48, 50, 52, 54, 56, 58, 60, 62 and 64; and/or the second light chain variable region (VLb) of the third polypeptide comprises the amino acid sequence of any one of SEQ ID NOS:16, 18, 20, 41, 43, 45, 47, 49, 51, 53, 55, 57, 59, 61, 63 and 65.

In one embodiment, the first heavy chain variable region (VHa) of the first polypeptide comprises the amino acid sequence of any one of SEQ ID NOS:22, 28, 30, 32, 34, 36 and 38 and the first light chain variable region (VLa) of the first polypeptide comprises the amino acid sequence of any one of SEQ ID NOS:24, 29, 31, 33, 35, 37 and 39; and the second heavy chain variable region (VHb) of the second polypeptide comprises the amino acid sequence of any one of SEQ ID NOS:1, 6, 8, 10, 12, 14, 40, 42, 44, 46, 48, 50, 52, 54, 56, 58, 60, 62 and 64; and the second light chain variable region (VLb) of the third polypeptide comprises the amino acid sequence of any one of SEQ ID NOS:16, 18, 20, 41, 43, 45, 47, 49, 51, 53, 55, 57, 59, 61, 63 and 65

The present disclosure provides a heterodimeric antibody comprising three polypeptide chains, wherein a first polypeptide comprises an amino acid sequence that is at least 95% identical to the amino acid sequence of SEQ ID NO:87, and a second polypeptide comprises an amino acid sequence that is at least 95% identical to the amino acid sequence of SEQ ID NO:89, and a third polypeptide comprises an amino acid sequence that is at least 95% identical to the amino acid sequence of SEQ ID NO:88 (e.g., BZ1).

The present disclosure provides a heterodimeric antibody comprising three polypeptide chains, wherein a first polypeptide comprises an amino acid sequence that is at least 95% identical to the amino acid sequence of SEQ ID NO:90, and a second polypeptide comprises an amino acid sequence that is at least 95% identical to the amino acid sequence of SEQ ID NO:92, and a third polypeptide comprises an amino acid sequence that is at least 95% identical to the amino acid sequence of SEQ ID NO:91 (e.g., BZ1S).

The present disclosure provides a heterodimeric antibody comprising three polypeptide chains, wherein a first polypeptide comprises an amino acid sequence that is at least 95% identical to the amino acid sequence of any one of SEQ ID NOs:71-77, and a second polypeptide comprises an amino acid sequence that is at least 95% identical to the amino acid sequence of SEQ ID NO:79, and a third polypeptide comprises an amino acid sequence that is at least 95% identical to the amino acid sequence of SEQ ID NO:85 (e.g., CD38 A2 series).

The present disclosure provides a heterodimeric antibody comprises three polypeptide chains, wherein a first polypeptide comprises an amino acid sequence that is at least 95% identical to the amino acid sequence of any one of SEQ ID NOs:71-77, and a second polypeptide comprises an amino acid sequence that is at least 95% identical to the amino acid sequence of SEQ ID NO:78, and a third polypeptide comprises an amino acid sequence that is at least 95% identical to the amino acid sequence of SEQ ID NO:84 (e.g., CD38 A2-3H10m1 series).

The present disclosure provides a heterodimeric antibody comprises three polypeptide chains, wherein a first polypeptide comprises an amino acid sequence that is at least 95% identical to the amino acid sequence of any one of SEQ ID NOs:71-77, and a second polypeptide comprises an amino acid sequence that is at least 95% identical to the amino acid sequence of any one of SEQ ID NOS:80-83, and a third polypeptide comprises an amino acid sequence that is at least 95% identical to the amino acid sequence of SEQ ID NO:86 (e.g., CD38 D8 series).

The present disclosure provide a heterodimeric antibody comprises three polypeptide chains, wherein a first polypeptide comprises an amino acid sequence that is at least 95% identical to the amino acid sequence of SEQ ID NO:100, and a second polypeptide comprises an amino acid sequence that is at least 95% identical to the amino acid sequence of SEQ ID NO:106, and a third polypeptide comprises an amino acid sequence that is at least 95% identical to the amino acid sequence of SEQ ID NO:86 (e.g., CD38 D8 bump-in-dent series).

In one embodiment, the first polypeptide comprises the amino acid sequence of SEQ ID NO:87, the second polypeptide comprises the amino acid sequence of SEQ ID NO:89, and the third polypeptide comprises the amino acid sequence of SEQ ID NO:88 (e.g., BZ1).

In one embodiment, the first polypeptide comprises the amino acid sequence of SEQ ID NO:90, the second polypeptide comprises the amino acid sequence of SEQ ID NO:92, and the third polypeptide comprises the amino acid sequence of SEQ ID NO:91 (e.g., BZ1S).

In one embodiment, the peptide linker of the first polypeptide comprises a flexible peptide linker.

In one embodiment, the peptide linker of the first polypeptide comprises the amino acid sequence of SEQ ID NO:23.

In one embodiment, the first hinge region of the first polypeptide comprises an IgG1, IgG2, IgG3 or IgG4 hinge region.

In one embodiment, the first hinge region of the first polypeptide comprises the amino acid sequence of SEQ ID NO:25.

In one embodiment, the second hinge region of the second polypeptide comprises an IgG1, IgG2, IgG3 or IgG4 hinge region.

In one embodiment, the second hinge region of the second polypeptide comprises the amino acid sequence of SEQ ID NO:3.

In one embodiment, the first Fc region of the first polypeptide and the second Fc region of the second polypeptide form an Fc domain that can bind Fc cell surface receptors.

In one embodiment, the first and second Fc regions form an Fc domain that exhibits effector function including complement-dependent cytotoxicity (CDC), antibody-dependent cell-mediated cytotoxicity (ADCC), antibody-dependent phagocytosis (ADP), opsonization and/or cell binding.

In one embodiment, the first and/or second Fc regions include a mutation that reduces Fc effector function.

In one embodiment, the first and/or second Fc regions include a LALA mutation.

In one embodiment, the first and second Fc regions are mutated to form knob-in-hole structures.

In one embodiment, the first and second Fc regions are mutated to form bump-in-dent structures.

In one embodiment, the first Fc region of the first polypeptide comprises the amino acid sequences of SEQ ID NOS:26 and 27.

In one embodiment, the second Fc region of the second polypeptide comprises the amino acid sequences of SEQ ID NOS:4 and 5.

In one embodiment, the first heavy chain variable region (VHa) and/or the first light chain variable region (VLa) of the first polypeptide include one or more point mutations that confer increased thermal-stability to the first polypeptide.

The present disclosure provides one or more nucleic acids encoding the first, second, and third polypeptides of any of the heterodimeric antibodies disclosed herein. The present disclosure provides one or more vectors comprising the one or more nucleic acids encoding the first, second, and third polypeptides of any of the heterodimeric antibodies disclosed herein. In some embodiments, the one or more vectors comprise a first and second vector. In some embodiments, the first vector encodes the first and second polypeptides, and the second vector encodes the third polypeptide. In some embodiments, the first vector encodes the second and third polypeptides, and the second vector encodes the first polypeptide. In some embodiments, the first vector encodes the first and third polypeptides, and the second vector encodes the second polypeptide.

The present disclosure provides a first nucleic acid encoding the first polypeptide.

The present disclosure provides a second nucleic acid encoding the second polypeptide.

The present disclosure provides a third nucleic acid encoding the third polypeptide.

The present disclosure provides a first nucleic acid encoding the first polypeptide, and a second nucleic acid encoding the second polypeptide, and a third nucleic acid encoding the third polypeptide.

The present disclosure provides a first vector (e.g, a first expression vector) comprising the first nucleic acid which encodes the first polypeptide.

The present disclosure provides a second vector (e.g, a second expression vector) comprising the second nucleic acid which encodes the second polypeptide.

The present disclosure provides a third vector (e.g, a third expression vector) comprising the third nucleic acid which encodes the third polypeptide.

The present disclosure provides a first vector (e.g., a first expression vector) comprising (i) the first nucleic acid which encodes the first polypeptide and (ii) the second nucleic acid of which encodes the second polypeptide, and a second vector (e.g., second expression vector) comprising the third nucleic acid which encodes the third polypeptide.

The present disclosure provides a first vector (e.g., a first expression vector) comprising (i) the second nucleic acid which encodes the second polypeptides and (ii) the third nucleic acid which encodes the third polypeptide, and a second vector (e.g., second expression vector) comprising the first nucleic acid which encodes the first polypeptide.

The present disclosure provides a first vector (e.g., a first expression vector) comprising the (i) first nucleic acid which encodes the first polypeptide and (ii) the third nucleic acid which encodes the third polypeptide, and a second vector (e.g., second expression vector) comprising the second nucleic acid which encodes the second polypeptide.

The present disclosure provides a vector (e.g., an expression vector) comprising the first nucleic acid which encodes the first polypeptide, the second nucleic acid which encodes the second polypeptide, and the third nucleic acid which encodes the third polypeptide.

The present disclosure provides a host cell, or a population of host cells, harboring any of the one or more nucleic acids or vectors disclosed herein.

The present disclosure provides a host cell, or a population of host cells, harboring the first expression vector which comprises the first nucleic acid encoding the first polypeptide.

The present disclosure provides host cell, or a population of host cells, harboring the second expression vector which comprises the second nucleic acid encoding the second polypeptide.

The present disclosure provides a host cell, or a population of host cells, harboring the third expression vector which comprises the third nucleic acid encoding the third polypeptide.

The present disclosure provides a host cell, or a population of host cells, harboring the first and second expression vectors, wherein the first expression vector comprises (i) the first nucleic acid encoding the first polypeptide and (ii) the second nucleic acid encoding the second polypeptide, and wherein the second expression vector comprises the third nucleic acid encoding the third polypeptide.

The present disclosure provides a host cell, or a population of host cells, harboring the first and second expression vectors, wherein the first expression vector comprises (i) the second nucleic acid encoding the second polypeptide and (ii) the third nucleic acid encoding the third polypeptide, and wherein the second expression vector comprises the first nucleic acid encoding the first polypeptide.

The present disclosure provides a host cell, or a population of host cells, harboring the first and second expression vectors, wherein the first expression vector comprises (i) the first nucleic acid encoding the first polypeptide and (ii) the third nucleic acid encoding the third polypeptide, and wherein the second expression vector comprises the second nucleic acid encoding the second polypeptide.

The present disclosure provides a host cell, or a population of host cells, harboring the expression vector which comprises (i) the first nucleic acid encoding the first polypeptide and (ii) the second nucleic acid encoding the second polypeptide, (iii) the third nucleic acid encoding the third polypeptide.

The present disclosure provides a method for preparing a heterodimeric antibody, comprising: culturing a population of host cells under conditions suitable for expressing the first, second and third polypeptides by the population of host cells, wherein individual host cells in the population of host cells harbor one or more nucleic acids or one or more vectors disclosed herein that encode the heterodimeric antibody, optionally wherein the one or more vectors comprise (i) a first vector encoding the first polypeptide and the second polypeptide and (ii) a second vector encoding the third polypeptide.

The present disclosure provides a method for preparing a heterodimeric antibody, comprising: (a) culturing a first population of host cells under conditions suitable for expressing the first polypeptide by the first population of host cells, wherein individual host cells in the first population of host cells harbor the first vector which comprises the first nucleic acid encoding the first polypeptide; (b) culturing a second population of host cells under conditions suitable for expressing the second polypeptide by the second population of host cells, wherein individual host cells in the second population of host cells harbor the second vector which comprises the second nucleic acid encoding the second polypeptide; and (c) culturing a third population of host cells under conditions suitable for expressing the third polypeptide by the third population of host cells, wherein individual host cells in the third population of host cells harbor the third vector which comprises the third nucleic acid encoding the third polypeptide. In one embodiment, the method further comprises: isolating the first, second and third polypeptides from the first, second and third population of cells, respectively. In one embodiment, the method further comprises subjecting the first, second and third polypeptides to conditions suitable for associating the first, second and third polypeptides with each other to form the heterodimeric antibody.

The present disclosure provides a method for preparing a heterodimeric antibody, comprising: culturing a population of host cells under conditions suitable for expressing the first, second and third polypeptides by the population of host cells, wherein individual host cells in the population of host cells harbor the first and second vectors, wherein the first vector (e.g., a first expression vector) comprises (i) the first nucleic acid which encodes the first polypeptide and (ii) the second nucleic acid which encodes the second polypeptide, wherein the second vector comprises the third nucleic acid which encodes the third polypeptide. In one embodiment, the method further comprises isolating the first, second and third polypeptides from the population of cells. In one embodiment, the method further comprises subjecting the first, second and third polypeptides to conditions suitable for associating the first, second and third polypeptides with each other to form the heterodimeric antibody.

The present disclosure provides a method for preparing a heterodimeric antibody, comprising: culturing a population of host cells under conditions suitable for expressing the first, second and third polypeptides by the population of host cells, wherein individual host cells in the population of host cells harbor the first and second vectors, wherein the first vector (e.g., a first expression vector) comprises (i) the second nucleic acid which encodes the second polypeptide and (ii) the third nucleic acid which encodes the third polypeptide, wherein the second vector comprises the first nucleic acid which encodes the first polypeptide. In one embodiment, the method further comprises isolating the first, second and third polypeptides from the population of cells. In one embodiment, the method further comprises subjecting the first, second and third polypeptides to conditions suitable for associating the first, second and third polypeptides with each other to form the heterodimeric antibody.

The present disclosure provides a method for preparing a heterodimeric antibody, comprising: culturing a population of host cells under conditions suitable for expressing the first, second and third polypeptides by the population of host cells, wherein individual host cells in the population of host cells harbor the first and second vectors, wherein the first vector (e.g., a first expression vector) comprises (i) the first nucleic acid which encodes the first polypeptide and (ii) the third nucleic acid which encodes the third polypeptide, wherein the second vector comprises the second nucleic acid which encodes the second polypeptide. In one embodiment, the method further comprises isolating the first, second and third polypeptides from the population of cells. In one embodiment, the method further comprises subjecting the first, second and third polypeptides to conditions suitable for associating the first, second and third polypeptides with each other to form the heterodimeric antibody.

The present disclosure provides a method for preparing a heterodimeric antibody, comprising: culturing a population of host cells under conditions suitable for expressing the first, second and third polypeptides by the population of host cells, wherein individual host cells in the population of host cells harbor the vector which comprises (i) the first nucleic acid which encodes the first polypeptide, (ii) the second nucleic acid which encodes the second polypeptide, and (iii) the third nucleic acid which encodes the third polypeptide. In one embodiment, the method further comprises isolating the first, second and third polypeptides from the population of cells. In one embodiment, the method further comprises subjecting the first, second and third polypeptides to conditions suitable for associating the first, second and third polypeptides with each other to form the heterodimeric antibody.

The present disclosure provides a method for binding a tumor-associated antigen and a T cell receptor, comprising: contacting any of the heterodimeric antibody described herein with the tumor-associated antigen and the T cell receptor under conditions suitable for binding the heterodimeric antibody to the tumor-associated antigen and the T cell receptor. In one embodiment, the heterodimeric antibody is contacted with the tumor-associated antigen first and contacted with the T cell receptor second (e.g., sequentially). In one embodiment, the heterodimeric antibody is contacted with the T cell receptor first and contacted with the tumor-associated antigen second (e.g., sequentially). In one embodiment, the heterodimeric antibody is contacted with the tumor-associated antigen and the T cell receptor at the same time (e.g., essentially simultaneously).

In one embodiment, in the method for binding a tumor-associated antigen and a T cell receptor, the tumor-associated antigen is expressed by a cell from a cancer of the prostate, breast, ovary, head and neck, bladder, skin, colorectal, anus, rectum, pancreas, lung (including non-small cell lung and small cell lung cancers), brain, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis, or testis or from a leiomyoma, glioma, or glioblastoma.

In one embodiment, in the method for binding a tumor-associated antigen and a T cell receptor, the tumor-associated antigen is expressed by a hematologic cancer. In some embodiments, the hematologic cancer is a B chronic lymphocytic leukemia (B-CLL), B and T acute lymphocytic leukemia (LL), acute myeloid leukemia (AML), chronic lymphocytic leukemia (CLL), chronic myelogenous leukemia (CML), hairy cell leukemia (HCL), myeloproliferative disorder/neoplasm (MPDS), myelodysplasia syndrome, non-Hodgkin's lymphoma (NHL) including Burkitt's lymphoma (BL), Waldenstrom's Macroglobulinemia, mantle cell lymphoma, AIDS-related lymphoma, Hodgkin's Lymphoma (HL), T cell lymphoma (TCL), multiple myeloma (MM), plasma cell myeloma, plamocytoma, giant cell myeloma, heavy-chain myeloma, and light chain or Bence-Jones myeloma.

In one embodiment, in the method for binding a tumor-associated antigen and a T cell receptor, the T cell receptor is expressed by an effector T cell. In one embodiment, the effector T cell kills the tumor or cancer cell by mediating cytotoxic cell killing. In one embodiment, the binding the heterodimeric antibody to the tumor-associated antigen and the T cell receptor forms a cell synapse. In one embodiment, the T cell receptor is expressed on an effector T cell and the tumor-associate antigen is expressed by a tumor or cancer cell. In one embodiment, the effector T cell in the cell synapse kills the tumor or cancer cell by mediating cytotoxic cell killing.

The present disclosure provides a method for treating a disease in a subject, comprising: administering to the subject a therapeutically effective amount of any of the heterodimeric antibodies described herein. The present disclosure provides a use of any of the heterodimeric antibodies described herein for the manufacture of a medicament for treating a disease. The present disclosure provides any of the heterodimeric antibodies described herein for use in treating a disease.

In one embodiment, with respect to the method, use, or antibody for use for treating a disease in a subject, the disease comprises a cancer of the prostate, breast, ovary, head and neck, bladder, skin, colorectal, anus, rectum, pancreas, lung (including non-small cell lung and small cell lung cancers), brain, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis, or testis, or a leiomyoma, glioma, or glioblastoma.

In one embodiment, with respect to the method, use, or antibody for use for treating a disease in a subject, the disease comprises a hematologic cancer. The hematologic cancer may be a B chronic lymphocytic leukemia (B-CLL), B and T acute lymphocytic leukemia (LL), acute myeloid leukemia (AML), chronic lymphocytic leukemia (CLL), chronic myelogenous leukemia (CML), hairy cell leukemia (HCL), myeloproliferative disorder/neoplasm (MPDS), myelodysplasia syndrome, non-Hodgkin's lymphoma (NHL) including Burkitt's lymphoma (BL), Waldenstrom's Macroglobulinemia, mantle cell lymphoma, AIDS-related lymphoma, Hodgkin's Lymphoma (HL), T cell lymphoma (TCL), multiple myeloma (MM), plasma cell myeloma, plamocytoma, giant cell myeloma, heavy-chain myeloma, or light chain or Bence-Jones myeloma.

### DESCRIPTION OF THE FIGURES

FIG. 1 is a schematic showing a non-limiting embodiment of a heterodimeric antibody.

FIG. 2 is a SDS-PAGE gel image of Protein A affinity liquid chromatography-purified transient CHO-expressed CD38/CD3 bispecific antibody throughout iterations of protein engineering.

FIG. 3A shows a chromatogram generated from analytical size exclusion chromatography (ANSEC) of a sample of a heterodimeric antibody after protein A purification. The data shown is for bispecific antibody CD38A2-3H10/CD3tsm #1 knob-into-hole.

FIG. 3B shows a chromatogram generated from analytical size exclusion chromatography (ANSEC) of a sample of a heterodimeric antibody after protein A purification. The data shown is for bispecific antibody CD38D8/ss4×CD3 bump-into-dent.

FIG. 4A shows primary T cell binding curves of various thermal-stability engineered heterodimeric antibodies compared to the parent antibodies, Hum291 anti-CD3 mAb and anti-CD38 A2-3H10 mAb. The heterodimeric antibodies include: CD38 A2-3H10m1 assembled with CD3-scFv tsm #1, 2, 3 or 4; and CD38 A2-3H10m1 assembled with CD3-scFv LALA. The EC50 values of the engineered heterodimeric antibodies are similar to each other in their binding to CD3 antigen expressed on CD38-minus T cells.

FIG. 4B shows primary T cell binding curves of various thermal-stability engineered heterodimeric antibodies compared to the parent antibodies, Hum291 anti-CD3 mAb and anti-CD38 A2-3H10 mAb. The heterodimeric antibodies include: CD38 A2-3H10m1 assembled with CD3-scFv tsm #5, 6 or 7; and CD38 A2-3H10m1 assembled with CD3-scFv LALA. The EC50 values of the engineered heterodimeric antibodies are similar to each other in their binding to CD3 antigen expressed on CD38-minus T cells.

FIG. 5A is a sensorgram from an SPR analysis of a monoclonal antibody CD38 A2-3H10M1 IgG-SPPC.

FIG. 5B is a sensorgram from an SPR analysis of a bispecific antibody which comprises a first polypeptide chain having the amino acid sequence of SEQ ID NO:71 (CD3 scFv-Fc fusion, VH variant M48I/K67R, tsm #1) and a second polypeptide chain having the amino acid sequence of SEQ ID NO:78 (CD38 A2-3H10m1).

FIG. 5C is a sensorgram from an SPR analysis of a bispecific antibody which comprises a first polypeptide chain having the amino acid sequence of SEQ ID NO:71 (CD3 scFv-Fc fusion, VH variant M48I/K67R, tsm #1) and a second polypeptide chain having the amino acid sequence of SEQ ID NO:79 (CD38 A2).

FIG. 5D is a sensorgram from an SPR analysis of a bispecific antibody which comprises a first polypeptide chain having the amino acid sequence of SEQ ID NO:71 (CD3 scFv-Fc fusion, VH variant M48I/K67R, tsm #1) and a second polypeptide chain having the amino acid sequence of SEQ ID NO:81 (CD38 D8-3A1).

FIG. 5E is a sensorgram from an SPR analysis of a bispecific antibody which comprises a first polypeptide chain having the amino acid sequence of SEQ ID NO:71 (CD3 scFv-Fc fusion, VH variant M48I/K67R, tsm #1) and a second polypeptide chain having the amino acid sequence of SEQ ID NO:83 (CD38 D8-1B5).

FIG. 5F is a sensorgram from an SPR analysis of a bispecific antibody which comprises a first polypeptide chain having the amino acid sequence of SEQ ID NO:71 (CD3 scFv-Fc fusion, VH variant M48I/K67R, tsm #1) and a second polypeptide chain having the amino acid sequence of SEQ ID NO:80 (CD38 D8).

FIG. 5G is a sensorgram from an SPR analysis of a bispecific antibody which comprises a first polypeptide chain having the amino acid sequence of SEQ ID NO:71 (CD3 scFv-Fc fusion, VH variant M48I/K67R, tsm #1) and a second polypeptide chain having the amino acid sequence of SEQ ID NO:82 (CD38 D8-3E1).

FIG. 6 is a Table listing binding kinetics determined for various heterodimeric antibodies as determined by BiaCore analysis.

FIG. 7 shows a bar graph comparing CD38 expression levels of six different CD38(+) tumor cell lines as determined via flow cytometry.

FIG. 8A shows the results of an antibody-mediated tumor cell killing by un-stimulated human T cells. Bispecific antibodies redirects previously un-stimulated human T cells to kill RPMI8226 cells in a dose-dependent manner.

FIG. 8B shows the results of an antibody-mediated tumor cell killing by un-stimulated human T cells. Bispecific antibodies redirects previously un-stimulated human T cells to kill RPMI8226 cells in a dose-dependent manner.

FIG. 9A shows the ratio of CD4(+)/CD8(+) in a T cell population and the percent of CD56(+) NK cells in the donor PBMC.

FIG. 9B shows the % killing by CD38×CD3 bispecific antibody or Darzalex of IM-9 tumor cells mixed with human PBMCs from Donor 1, 2 or 4.

FIG. 9C shows the % killing by CD38×CD3 bispecific antibody or Darzalex of MM1.R tumor cells mixed with human PBMCs from Donor 1, 2 or 4.

FIG. 9D shows the % killing by CD38×CD3 bispecific antibody or Darzalex of NCI-H929 tumor cells mixed with human PBMCs from Donor 1, 2 or 4.

FIG. 9E shows the % killing by CD38×CD3 bispecific antibody or Darzalex of RPMI8226 tumor cells mixed with human PBMCs from Donor 1, 2 or 4.

FIG. 9F shows the % killing by CD38×CD3 bispecific antibody or Darzalex of Raji tumor cells mixed with human PBMCs from Donor 1, 2 or 4.

FIG. 9G shows the % killing by CD38×CD3 bispecific antibody or Darzalex of Daudi tumor cells mixed with human PBMCs from Donor 1, 2 or 4.

FIG. 10A shows the results of an in vitro IFNγ release assay of heterodimeric antibodies (CD38A/CD3 IgG-scFv bispecific antibody and control RSV/CD3 IgG-scFv bispecific antibody) compared to a combination of two monoclonal antibodies (CD38 mAb+CD3 mAb).

FIG. 10B shows the results of an in vitro TNFα release assay of heterodimeric antibodies (CD38A/CD3 IgG-scFv bispecific antibody and control RSV/CD3 IgG-scFv bispecific antibody) compared to a combination of two monoclonal antibodies (CD38 mAb+CD3 mAb).

FIG. 10C shows the results of an in vitro IL-2 release assay of heterodimeric antibodies (CD38A/CD3 IgG-scFv bispecific antibody and control RSV/CD3 IgG-scFv bispecific antibody) compared to a combination of two monoclonal antibodies (CD38 mAb+CD3 mAb).

FIG. 11A shows the results of an in vitro T cell activation assay of heterodimeric antibodies (CD38A2-3H10m1/CD3tsm #1-scFv and control RSV/CD3tsm #1-scFv) compared to a combination of two monoclonal antibodies (CD38 mAb+HuM291-CD3 mAb), at 1 pM or 10 pM or 1 nM antibody concentration, in the presence of MM1.R target tumor cells.

FIG. 11B shows the results of an in vitro T cell activation assay of heterodimeric antibodies (CD38A2-3H10m1/CD3tsm #1-scFv and control RSV/CD3tsm #1-scFv) compared to a combination of two monoclonal antibodies (CD38 mAb+HuM291-CD3 mAb), at 1 pM or 10 pM or 1 nM antibody concentration, in the absence of MM1.R target tumor cells.

FIG. 12A is a bar graph showing the results of a T cell activation assay. Bispecific antibody (CD38 A2-3H10m1/CD3 IgG-ScFv) mediated in vitro T cell activation in a dose-dependent manner. The bar graph shows normalized percent T cell activation obtained by subtracting the level of activation observed in target cells and T cells only conditions from FIGS. 9A and B.

FIG. 12B is a bar graph showing the results of a T cell activation assay. The combination of two parental mAbs (CD38 A2-3H10m1 and anti-CD3) mediated in vitro T cell activation in a dose-dependent manner.

FIG. 12C is a bar graph showing the results of a T cell activation assay. Control bispecific antibody (RSV/CD3 IgG-scFv) did not mediate in vitro T cell activation.

FIG. 13A is a graph showing the results of an ADCC assay using Daudi cells as a source of CD38-expressing target cells and NK cells prepared from PBMCs from donor 1.

FIG. 13B is a graph showing the results of an ADCC assay using Daudi cells as a source of CD38-expressing target cells and NK cells prepared from PBMCs from donor 2.

FIG. 14A shows a timeline for an in vivo anti-tumor murine study using mice engrafted with luciferase-tagged RPMI8226 MM disseminated tumor cells then administered T cells, treated three times with a control antibody or one of two different CD38/CD3 heterodimeric (bispecific) antibodies.

FIG. 14B is a table listing the treatment schedule for 9 groups of mice described in FIG. 14A.

FIG. 14C shows tumor burden (RPMI8226) using bioluminescence imaging of a xenograft murine model treated with a heterodimeric (bispecific-1) antibody.

FIG. 14D shows average bioluminescence signals from the treated mice shown in FIGS. 14A-C.

FIG. 14E shows average bioluminescence signals from the treated mice shown in FIGS. 14A-C.

FIG. 15A shows tumor burden (RPMI8226) using bioluminescence imaging of a xenograft murine model treated with a heterodimeric (bispecific-2) antibody. The bispecific-2 antibody has a different anti-CD38 arm compared to the bispecific-1 antibody represented in FIG. 14C.

FIG. 15B shows average bioluminescence signals from the treated mice shown in FIG. 15A.

FIG. 15C shows average bioluminescence signals from the treated mice shown in FIG. 15A.

FIG. 16A is a schematic showing a timeline for an in vivo anti-tumor murine study using mice engrafted with luciferase-tagged RPMI8226 tumor cells then administered T cells, treated three times with a control antibody or one of two different CD38/CD3 heterodimeric (bispecific) antibodies.

FIG. 16B is a table listing the treatment schedule for the mice as described in FIG. 16A.

FIG. 16C shows tumor burden using bioluminescence imaging of the xenograft murine model treated with the heterodimeric antibodies as described in FIGS. 16A and B.

FIG. 16D shows total flux of bioluminescence signals of tumor burden in the treated mice described in FIGS. 16A-C.

FIG. 16E is a table listing the mean and S.E.M of IVIS Average Radiance (unit: p/s/cm²/sr).

FIG. 16F is a graph showing the detection of human T cells (CD3+ T cells) in blood taken from the mice shown in FIG. 16C.

FIG. 16G is a graph showing T cell engagement 1 day after administration of CD38×CD3 bispecific antibody as detected in blood from the mice shown in FIG. 16C.

FIG. 16H is a graph showing T cell expansion 1 week after administration of CD38×CD3 bispecific antibody as detected in blood from the mice shown in FIG. 16C.

FIG. 16I is a table listing the mean and S.E.M. of human CD3+ cell detection (/uL).

FIG. 17A is a schematic showing a timeline for an in vivo anti-tumor murine study using mice engrafted with luciferase-tagged Raji tumor cells then administered T cells, treated three times with a control antibody or one of two different CD38/CD3 heterodimeric (bispecific) antibodies.

FIG. 17B shows tumor burden (Raji) using bioluminescence imaging of a xenograft murine model treated with a heterodimeric antibody (up to day 79).

FIG. 17C shows total flux of bioluminescence signals of tumor burden in the treated mice described in FIG. 17A.

FIG. 17D shows body weight change percent of the treated mic described in FIG. 17A.

FIG. 17E shows levels of CD45+ human T cells contained in 50 uL of mouse blood obtained on the days indicated in the graph as detected by flow cytometry. The engrafted human immune cells were stained with PE/Cy7 anti-CD3, APC/Cy7 anti-CD45, APC anti-CD38, PE anti-CD8a Antibody PerCP/Cyanine5.5 mouse anti-CD4 antibody.

FIG. 17F shows the CD3+% human T cells from CD45+ cells contained in 25 uL of mouse blood obtained on the days indicated in the graph as detected by flow cytometry.

FIG. 17G shows the CD4+% human T cells from CD3+ cells contained in 25 uL of mouse blood obtained on the days indicated in the graph as detected by flow cytometry.

FIG. 17H shows the CD8a+% human T cells from CD3+ cells contained in 25 uL of mouse blood obtained on the days indicated in the graph as detected by flow cytometry.

FIG. 17I shows the levels of CD38 MFI detected in C45+ cells contained in 25 uL of mouse blood obtained on the days indicated in the graph as detected by flow cytometry.

FIG. 17J is a graph showing animal survival analysis (Mayer survival curve) based on the data shown in FIG. 17A.

FIG. 18A is a schematic showing the two approaches tested for stable pool production of the heterodimeric antibodies.

FIG. 18B shows a Coomassie-stained SDS-PAGE gel of heterodimeric antibody expression from stable pool production. The solid star designates excess light chain. The solid arrow designates the heavy chain.

FIG. 18C shows an anti-human Fc Western blot analysis of the SDS-PAGE gel described in FIG. 18B.

FIG. 18D shows an anti-human Fc and anti-human lambda Western blot analysis of the SDS-PAGE gel described in FIG. 18B. The solid star designates excess light chain.

FIG. 18E shows the results of purifying one of the heterodimeric antibodies (BZ1) on a Capto™ S Impact column.

FIG. 18F shows the results of purifying one of the heterodimeric antibodies (BZ1S) on a Capto™ S Impact column.

FIG. 19A shows a UV trace and MS trace from LC-MS analysis of an isolated and purified bispecific antibody.

FIG. 19B shows the results of subjecting an isolated and purified bispecific antibody to CE-SDS under reducing conditions.

FIG. 19C shows the results of subjecting an isolated and purified bispecific antibody to CE-SDS under non-reducing conditions.

FIG. 19D shows a table listing the determined protein mass of the isolated and purified bispecific antibody using the CE-SDS results shown in FIGS. 19B and C.

FIG. 20A shows the results of subjecting an isolated and purified bispecific antibody (BZ1) to ion exchange chromatography under reducing conditions.

FIG. 20B shows the results of subjecting an isolated and purified bispecific antibody (BZ1) to ion exchange chromatography under non-reducing conditions.

FIG. 20C shows a table listing the determined protein mass of the isolated and purified bispecific antibody using the ion exchange chromatography results shown in FIGS. 20A and B.

FIG. 21A shows the results of subjecting an isolated and purified bispecific antibody (BZ1S) to ion exchange chromatography under reducing conditions.

FIG. 21B shows the results of subjecting an isolated and purified bispecific antibody (BZ1S) to ion exchange chromatography under non-reducing conditions.

FIG. 21C shows a table listing the determined protein mass of the isolated and purified bispecific antibody using the ion exchange chromatography results shown in FIGS. 21A and B.

FIG. 21D is an SDS-PAGE gel analysis of single cell clone supernatants of BZ1S and BZ1 bispecific antibodies. The stars designate the antibody clones that express predominantly correctly paired first, second and third polypeptides that make up the bispecific antibodies (lane 3: BZ1S-32; lane 9: BZ1-15; lane 10: BZ1-37; lane 11: BZ1-56; and lane 12: BZ1-71).

FIG. 22A is a sensorgram from an SPR analysis of a bispecific antibody BZ1 bispecific antibody.

FIG. 22B is a sensorgram from an SPR analysis of a bispecific antibody BZ1S bispecific antibody.

FIG. 22C is a sensorgram from an SPR analysis of a bispecific antibody BZ1 bispecific antibody.

FIG. 22D is a table listing binding kinetic values from the sensorgrams obtained from the SPR analysis shown in FIGS. 22A-C.

### DETAILED DESCRIPTION

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to cleavage, for example cleavage by a secretory signal peptide or by non-enzymatic cleavage at certain amino acid residues. Polypeptides include mature molecules that have undergone cleavage. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins. Two or more polypeptides (e.g., 3 polypeptide chains) can associate with each other, via covalent and/or non-covalent association, to form a polypeptide complex. Association of the polypeptide chains can also include peptide folding. Thus, a polypeptide complex can be dimeric, trimeric, tetrameric, or higher order complexes depending on the number of polypeptide chains that form the complex.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding an antibody, or a fragment or scFv, derivative, mutein, or variant thereof. In one embodiment, nucleic acids comprise one type of polynucleotide or a mixture of two or more different types of polynucleotides. Nucleic acids encoding the heterodimeric antibodies, are described herein.

The term “recover” or “recovery” or “recovering”, and other related terms, refers to obtaining a protein (e.g., an antibody or an antigen binding portion thereof), from host cell culture medium or from host cell lysate or from the host cell membrane. In one embodiment, the protein is expressed by the host cell as a recombinant protein fused to a secretion signal peptide (leader peptide sequence) sequence which mediates secretion of the expressed protein from a host cell (e.g., from a mammalian host cell). The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or G (cell wall components from Staphylococcus aureus).

The term “isolated” refers to a protein (e.g., an antibody or an antigen binding portion thereof) or polynucleotide that is substantially free of other cellular material. A protein may be rendered substantially free of naturally associated components (or components associated with a cellular expression system or chemical synthesis methods used to produce the antibody) by isolation, using protein purification techniques well known in the art. The term isolated also refers to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrometry. In one embodiment, the heterodimeric antibodies, of the present disclosure are isolated.

The term “leader sequence” or “leader peptide” or “peptide signal sequence” or “signal peptide” or “secretion signal peptide” refers to a peptide sequence that is located at the N-terminus of a polypeptide. A leader sequence directs a polypeptide chain to a cellular secretory pathway and can direct integration and anchoring of the polypeptide into the lipid bilayer of the cellular membrane. Typically, a leader sequence is about 10-50 amino acids in length. A leader sequence can direct transport of a precursor polypeptide from the cytosol to the endoplasmic reticulum. In one embodiment, a leader sequence includes signal sequences comprising CD8a, CD28 or CD16 leader sequences. In one embodiment, the signal sequence comprises a mammalian sequence, including for example mouse or human Ig gamma secretion signal peptide. In one embodiment, a leader sequence comprises a mouse Ig gamma leader peptide sequence MEWSWVFLFFLSVTTGVHS (SEQ ID NO: 111).

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold. In one embodiment, the heterodimeric antibodies, of the present disclosure behave like antigen binding proteins that bind two different target antigens and are described herein.

An antigen binding protein can have, for example, the structure of a naturally occurring immunoglobulin. In one embodiment, an “immunoglobulin” refers to a naturally-occurring tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The heavy and/or light chains may or may not include a leader sequence for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules. In one embodiment, the heterodimeric antibodies of the present disclosure exhibit immunoglobulin-like properties that bind specifically to two different target antigens and are described herein.

The variable regions of immunoglobulin chains exhibit the same general structure of relatively conserved framework regions (FR) joined by three hypervariable regions, also called complementarity determining regions or CDRs. From N-terminus to C-terminus, both light and heavy chain variable regions comprise the domains FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5^{th} Ed., US Dept. of Health and Human Services, PHS, NIH, NIH Publication no. 91-3242, 1991 (“Kabat numbering”). Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations. In one embodiment, the heterodimeric antibodies of the present disclosure behave like antigen binding proteins that bind two different target antigens and are described herein.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains. Antigen binding domains from heterodimeric antibodies are described herein.

The terms “specific binding”, “specifically binds” or “specifically binding” and other related terms, as used herein in the context of an antibody or antigen binding protein (e.g., heterodimeric antibody) or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less, or 10⁻¹¹ M or less. Heterodimeric antibodies that specifically bind CD38 and CD3 are described herein.

In one embodiment, binding specificity can be measure by ELISA, radioimmune assay (MA), electrochemiluminescence assays (ECL), immunoradiometric assay (IRMA), or enzyme immune assay (EIA).

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, N.J.).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. Heterodimeric antibodies that bind an epitope of a CD38 polypeptide and that bind an epitope of a CD3 polypeptide are described herein.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or V_{L} domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989). Fab fragments comprising antigen binding portions from a heterodimeric antibody are described herein.

A single-chain antibody (scFv) is an antibody in which a V_{L} and a V_{H} region are joined via a linker (e.g., a synthetic sequence of amino acid residues) to form a continuous protein chain capable of forming a monovalent antigen binding site (see, e.g., Bird et al., 1988, Science 242:423-26 and Huston et al., 1988, Proc. Natl. Acad. Sci. USA 85:5879-83). Single chain antibodies comprising antigen binding portions from heterodimeric antibodies are described herein.

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different. Diabody, tribody and tetrabody constructs can be prepared using antigen binding portions from any of the heterodimeric antibodies described herein.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes. Fully human heterodimeric antibodies and antigen binding proteins thereof are described herein.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293. Heterodimeric antibodies having humanized portions are described herein.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen). Heterodimeric antibodies having chimeric portions are described herein.

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 domains, and may or may not include a portion of the hinge region. Two polypeptide chains each containing an Fc region can dimerize to form a dimeric Fc region. In some embodiments, a dimeric Fc region can bind Fc cell surface receptors and some proteins of the immune complement system. In some embodiments, a dimeric Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. In some embodiments, a dimeric Fc region can bind an Fc receptor, including FcγRI (e.g., CD64), FcγRII (e.g., CD32) and/or FcγRIII (e.g., CD16a).

The term “labeled antibody” or related terms as used herein refers to antibodies and their antigen binding portions thereof that are unlabeled or joined to a detectable label or moiety for detection, wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the heterodimeric antibodies described herein can be unlabeled or can be joined to a detectable label or moiety.

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of a test antibody may be similar but not identical to any of the amino acid sequences of the polypeptides that make up the heterodimeric antibodies described herein. The similarities between the test antibody and the polypeptides can be at least 95%, or at or at least 96% identical, or at least 97% identical, or at least 98% identical, or at least 99% identical, to any of the polypeptides that make up the heterodimeric antibodies described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules. Vectors can be linear or circular nucleic acid molecules. A donor nucleic acid used for gene editing methods employing zinc finger nuclease, TALEN or CRISPR/Cas can be a type of a vector. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Expression vectors can include one or more origin of replication sequence. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606. An expression vector can comprise nucleic acids that encode at least a portion of any of the heterodimeric antibodies described herein.

A transgene is “operably linked” to a vector when there is linkage between the transgene and the vector to permit functioning or expression of the transgene sequences contained in the vector. In one embodiment, a transgene is “operably linked” to a regulatory sequence when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one which has been transfected, transformed or transduced with exogenous nucleic acid (transgene). The host cell includes the primary subject cell and its progeny. Exogenous nucleic acids encoding at least a portion of any of the heterodimeric antibodies described herein can be introduced into a host cell. Expression vectors comprising at least a portion of any of the heterodimeric antibodies described herein can be introduced into a host cell, and the host cell can express polypeptides comprising at least a portion of the heterodimeric antibodies.

The terms “host cell” or “or a population of host cells” or related terms as used herein refer to a cell (or a population thereof) into which foreign (exogenous or transgene) nucleic acids have been introduced. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell or can be extracted from a subject. The host cell (or a population thereof) includes the primary subject cell and its progeny without any regard for the number of passages. Progeny cells may or may not harbor identical genetic material compared to the parent cell. Host cells encompass progeny cells. In one embodiment, a host cell describes any cell (including its progeny) that has been modified, transfected, transduced, transformed, and/or manipulated in any way to express an antibody, as disclosed herein. In one example, the host cell (or population thereof) can be introduced with an expression vector operably linked to a nucleic acid encoding the desired antibody, or an antigen binding portion thereof, described herein. Host cells and populations thereof can harbor an expression vector that is stably integrated into the host's genome, or can harbor an extrachromosomal expression vector. In one embodiment, host cells and populations thereof can harbor an extrachromosomal vector that is present after several cell divisions or is present transiently and is lost after several cell divisions.

Transgenic host cells can be prepared using non-viral methods, including well-known designer nucleases including zinc finger nucleases, TALENS or CRISPR/Cas. A transgene can be introduced into a host cell's genome using genome editing technologies such as zinc finger nuclease. A zinc finger nuclease includes a pair of chimeric proteins each containing a non-specific endonuclease domain of a restriction endonuclease (e.g., FokI) fused to a DNA-binding domain from an engineered zinc finger motif. The DNA-binding domain can be engineered to bind a specific sequence in the host's genome and the endonuclease domain makes a double-stranded cut. The donor DNA carries the transgene, for example any of the nucleic acids encoding a CAR or DAR construct described herein, and flanking sequences that are homologous to the regions on either side of the intended insertion site in the host cell's genome. The host cell's DNA repair machinery enables precise insertion of the transgene by homologous DNA repair. Transgenic mammalian host cells have been prepared using zinc finger nucleases (U.S. Pat. Nos. 9,597,357, 9,616,090, 9,816,074 and 8,945,868). A transgenic host cell can be prepared using TALEN (Transcription Activator-Like Effector Nucleases) which are similar to zinc finger nucleases in that they include a non-specific endonuclease domain fused to a DNA-binding domain which can deliver precise transgene insertion. Like zinc finger nucleases, TALEN also introduce a double-strand cut into the host's DNA. Transgenic host cells can be prepared using CRISPR (Clustered Regularly Interspaced Short Palindromic Repeats). CRISPR employs a Cas endonuclease coupled to a guide RNA for target specific donor DNA integration. The guide RNA includes a conserved multi-nucleotide containing protospacer adjacent motif (PAM) sequence upstream of the gRNA-binding region in the target DNA and hybridizes to the host cell target site where the Cas endonuclease cleaves the double-stranded target DNA. The guide RNA can be designed to hybridize to a specific target site. Similar to zinc finger nuclease and TALEN, the CRISPR/Cas system can be used to introduce site specific insertion of donor DNA having flanking sequences that have homology to the insertion site. Examples of CRISPR/Cas systems used to modify genomes are described for example in U.S. Pat. Nos. 8,697,359, 10,000,772, 9,790,490, and U. S. Patent Application Publication No. US 2018/0346927. In one embodiment, transgenic host cells can be prepared using zinc finger nuclease, TALEN or CRISPR/Cas system, and the host target site can be a TRAC gene (T Cell Receptor Alpha Constant). The donor DNA can include for example any of the nucleic acids encoding a CAR or DAR construct described herein. Electroporation, nucleofection or lipofection can be used to co-deliver into the host cell the donor DNA with the zinc finger nuclease, TALEN or CRISPR/Cas system.

A host cell can be a prokaryote, for example, E. coli, or it can be a eukaryote, for example, a single-celled eukaryote (e.g., a yeast or other fungus), a plant cell (e.g., a tobacco or tomato plant cell), an mammalian cell (e.g., a human cell, a monkey cell, a hamster cell, a rat cell, a mouse cell, or an insect cell) or a hybridoma. In one embodiment, a host cell can be introduced with an expression vector operably linked to a nucleic acid encoding a desired antibody thereby generating a transfected/transformed host cell which is cultured under conditions suitable for expression of the antibody by the transfected/transformed host cell, and optionally recovering the antibody from the transfected/transformed host cells (e.g., recovery from host cell lysate) or recovery from the culture medium. In one embodiment, host cells comprise non-human cells including CHO, BHK, NS0, SP2/0, and YB2/0. In one embodiment, host cells comprise human cells including HEK293, HT-1080, Huh-7 and PER.C6. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20. In one embodiment, a host cell is a mammalian host cell, but is not a human host cell. Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein. A host cell, or a population of host cells, harboring a vector (e.g., an expression vector) operably linked to at least one nucleic acid encoding one or more heterodimeric antibodies are described herein.

Polypeptides of the present disclosure (e.g., antibodies and antigen binding proteins) can be produced using any method known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides disclosed herein would make expression in E. coli as the preferred method for expression. The protein is then purified from culture media or cell extracts. Any of the heterodimeric antibodies can be expressed by transgenic host cells.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein are preferably at least 65% pure, at least 75% pure, at least 85% pure, more preferably at least 95% pure, and most preferably at least 98% pure. Regardless of the exact numerical value of the purity, the polypeptide is sufficiently pure for use as a pharmaceutical product. Any of the heterodimeric antibodies described herein can be expressed by transgenic host cells and then purified to about 65-98% purity or high level of purity using any art-known method.

In certain embodiments, the antibodies and antigen binding proteins herein can further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. A preferred form of glycosylation is sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

In one embodiment, the antibodies and antigen binding proteins described herein can be modified to become soluble polypeptides which comprises linking the Antibodies and antigen binding proteins to non-proteinaceous polymers. In one embodiment, the non-proteinaceous polymer comprises polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337.

PEG is a water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ—CH₂CH₂OH (1), where n is 20 to 2300 and X is H or a terminal modification, e.g., a Ci-4 alkyl. In one embodiment, the PEG terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462. One form of PEGs includes two PEG side-chains (PEG2) linked via the primary amino groups of a lysine (Monfardini et al., Bioconjugate Chem. 6 (1995) 62-69).

The serum clearance rate of PEG-modified polypeptide may be modulated (e.g., increased or decreased) by about 10%, 20%, 30%, 40%, 50%, 60%, 70%, 80%, or even 90%, relative to the clearance rate of the unmodified antibodies and antigen binding proteins binding polypeptides. The PEG-modified antibodies and antigen binding proteins may have a half-life (t_{1/2}) which is enhanced relative to the half-life of the unmodified polypeptide. The half-life of PEG-modified polypeptide may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified antibodies and antigen binding proteins. In one embodiment, the protein half-life is determined in vitro, such as in a buffered saline solution or in serum. In other embodiments, the protein half-life is an in vivo half-life, such as the half-life of the protein in the serum or other bodily fluid of an animal.

The present disclosure provides therapeutic compositions comprising any of the heterodimeric antibodies described herein in an admixture with a pharmaceutically-acceptable excipient. An excipient encompasses carriers, stabilizers and excipients. Excipients of pharmaceutically acceptable excipients includes for example inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, non-ionic detergents, anti-oxidants and isotonifiers.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

Any of the heterodimeric antibodies (or antigen binding protein thereof) may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the antibody (or antigen binding protein thereof) is formulated in the presence of sodium acetate to increase thermal stability.

Any of the heterodimeric antibodies (or antigen binding protein thereof) may be formulated for oral use include tablets containing the active ingredient(s) in a mixture with non-toxic pharmaceutically acceptable excipients. Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Any of the heterodimeric antibodies described herein (or antigen binding protein thereof) can be administered to a subject using art-known methods and delivery routes.

The terms “effective amount”, “therapeutically effective amount” or “effective dose” or related terms may be used interchangeably and refer to an amount of antibody or an antigen binding protein (e.g., heterodimeric antibodies) that when administered to a subject, is sufficient to effect a measurable improvement or prevention of a disease or disorder associated with tumor or cancer antigen expression. Therapeutically effective amounts of antibodies provided herein, when used alone or in combination, will vary depending upon the relative activity of the antibodies and combinations (e.g., in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, the polypeptide is administered at about 0.01 g/kg to about 50 mg/kg per day, preferably 0.01 mg/kg to about 30 mg/kg per day, most preferably 0.1 mg/kg to about 20 mg/kg per day. The polypeptide may be administered daily (e.g., once, twice, three times, or four times daily) or preferably less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

The present disclosure provides methods for treating a subject having a disease associated with expression of one or more tumor-associated antigens. The disease comprises cancer or tumor cells expressing the tumor-associated antigens, such as for example CD38 and/or CD3 antigen. In one embodiment, the cancer or tumor includes cancer of the prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis and testis.

In one embodiment, the cancer comprises hematological cancers, including leukemias, lymphomas, myelomas and B cell lymphomas. Hematologic cancers include multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CML), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, primary, or immunocyte-associated amyloidosis, and monoclonal gammopathy of undetermined significance.

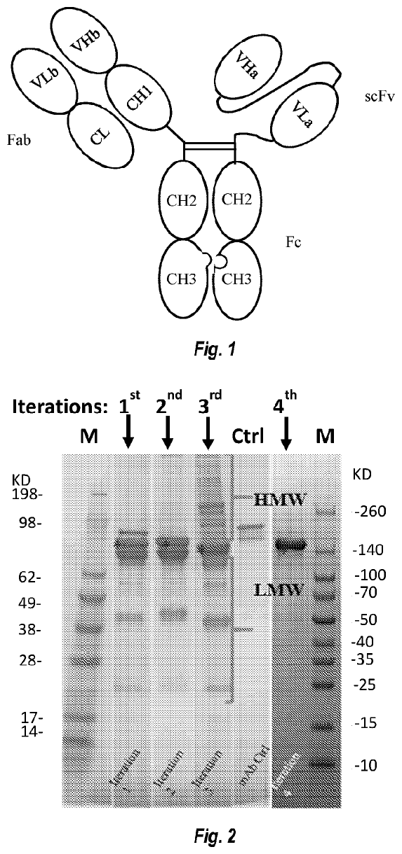

The present disclosure provides heterodimeric antibodies that bind to two different target antigens at the same time. In one embodiment, the heterodimeric antibodies are bispecific antibodies. In one embodiment, the heterodimeric antibodies comprise three polypeptides including: a first polypeptide comprising an scFv-Fc fusion polypeptide; a second polypeptide comprising an immunoglobulin heavy chain; and a third polypeptide comprising an immunoglobulin light chain (FIG. 1). In one embodiment, the first polypeptide includes one or more point mutations that confer increased thermal-stability, reduced Fc effector function and/or increased heterodimerization capability. In one embodiment, the second polypeptide includes one or more point mutations that confer reduced Fc effector function and/or increased heterodimerization capability.

The present disclosure provides a heterodimeric antibody that binds a T cell receptor and a tumor-associated antigen. In one embodiment, the heterodimer binds a T cell receptor comprising a CD3-epsilon antigen from human (e.g., SEQ ID NO:93, UniProt P07766-1). In one embodiment, the heterodimeric antibody cross-reacts with a CD3-epsilon antigen from a non-human animal, including mouse, rat, goat, rabbit, hamster and/or monkey (e.g., cynomolgus, rhesus or macaque). In one embodiment, the heterodimeric antibody binds a tumor-associated antigen comprising a CD38 antigen from human (e.g., SEQ ID NO:70, UniProt P28907). In one embodiment, the heterodimeric antibody cross-reacts with a CD38 antigen from a non-human animal, including mouse, rat, goat, rabbit, hamster and/or monkey (e.g., cynomolgus, rhesus or macaque). In one embodiment, the heterodimeric antibody cross-reacts with CD38 from cynomolgus monkey (SEQ ID NO:107, UniProt Q5VAN0), CD38 from mouse (SEQ ID NO:108, UniProt P565280) and/or CD38 from rat (SEQ ID NO:109, UniProt Q64244).

The present disclosure provides a heterodimeric antibody that binds a tumor-associated antigen and a T cell receptor, the antibody comprising: (a) a first polypeptide comprising an scFv and a first Fc region, wherein the scFv forms a first antigen binding domain that binds the T cell receptor; (b) a second polypeptide comprising a heavy chain variable region and a second Fc region; and (c) a third polypeptide comprising a light chain variable region, wherein the heavy chain variable region and the light chain variable region form a second antigen binding domain that binds the tumor-associated antigen. In one embodiment, the tumor-associated antigen comprises a CD38 antigen. In one embodiment, the CD38 antigen comprises the amino acid sequence of SEQ ID NO:70. In one embodiment, the CD38 antigen comprises the amino acid sequence of SEQ ID NO:107, 108 and/or 109. In one embodiment the T cell receptor comprises a CD3 antigen. In one embodiment, the first heavy chain variable region (VHa) and/or the first light chain variable region (VLa) of the first polypeptide include one or more point mutations that confer increased thermal-stability to the first polypeptide.

The present disclosure provides a heterodimeric antibody that binds a tumor-associated antigen and a T cell receptor, the antibody comprising: (a) a first polypeptide comprising an scFv and a first Fc region, wherein the scFv includes, in order from N-terminus to C-terminus, a first heavy chain variable region (VHa) and a first light chain variable region (VLa) joined together by a peptide linker, and wherein the first heavy chain variable region (VHa) and a first light chain variable region (VLa) form a first antigen binding domain that binds the T cell receptor; (b) a second polypeptide comprising a second heavy chain variable region (VHb) and a second Fc region; and (c) a third polypeptide comprising a second light chain variable region (VLb), wherein the second heavy chain variable region (VHb) and the second light chain variable region (VLb) form a second antigen binding domain that binds the tumor-associated antigen.

In one embodiment, the first polypeptide comprises an scFv and a first Fc region, wherein the scFv includes, in order from N-terminus to C-terminus, a first light chain variable region (VLa) and a first heavy chain variable region (VHa) joined together by a peptide linker, and wherein the first light chain variable region (VLa) and a first heavy chain variable region (VHa) form a first antigen binding domain that binds the T cell receptor.

In one embodiment, the first polypeptide comprising an scFv and a first Fc region, wherein the scFv includes a first heavy chain variable region (VHa) and a first light chain variable region (VLa) and joined together by a peptide linker, and wherein the first heavy chain variable region (VHa) and the first light chain variable region (VLa) form a first antigen binding domain that binds the T cell receptor. In one embodiment, the tumor-associated antigen comprises a CD38 antigen. In one embodiment, the CD38 antigen comprises the amino acid sequence of SEQ ID NO:70. In one embodiment, the CD38 antigen comprises the amino acid sequence of SEQ ID NO:107, 108 and/or 109. In one embodiment the T cell receptor comprises a CD3 antigen. In one embodiment, the first heavy chain variable region (VHa) and/or the first light chain variable region (VLa) of the first polypeptide include one or more point mutations that confer increased thermal-stability to the first polypeptide.

The present disclosure provides a heterodimeric antibody that binds a tumor-associated antigen and a T cell receptor, the antibody comprising: (a) a first polypeptide comprising an scFv and a first Fc region, wherein the scFv includes a first heavy chain variable region and a first light chain variable region joined together by a peptide linker, wherein the scFv is joined to the first Fc by a first hinge region, and wherein the first heavy chain variable region and a first light chain variable region form a first antigen binding domain that binds the T cell receptor; (b) a second polypeptide comprising a second heavy chain variable region and a second Fc region, wherein the second heavy chain variable region is joined to the second Fc region by a second hinge region, and wherein the first and second hinge regions form a disulfide bond; and (c) a third polypeptide comprising a second light chain variable region, wherein the second heavy chain variable region and the second light chain variable region form a second antigen binding domain that binds the tumor-associated antigen. In one embodiment, the tumor-associated antigen comprises a CD38 antigen. In one embodiment, the CD38 antigen comprises the amino acid sequence of SEQ ID NO:70. In one embodiment, the CD38 antigen comprises the amino acid sequence of SEQ ID NO:107, 108 and/or 109. In one embodiment the T cell receptor comprises a CD3 antigen. In one embodiment, the CD3 antigen comprises a human CD3 epsilon antigen having the amino acid sequence of SEQ ID NO:93. In one embodiment, the first heavy chain variable region (VHa) and/or the first light chain variable region (VLa) of the first polypeptide include one or more point mutations that confer increased thermal-stability to the first polypeptide.

The present disclosure provides a heterodimeric antibody comprising a first polypeptide chain which is an scFv-Fc fusion polypeptide which comprises a first heavy chain variable region (VHa) and the first light chain variable region (VLa), wherein the VHa region comprises humanized or fully human immunoglobulin sequences.

In one embodiment, the heterodimeric antibody comprises a first polypeptide chain comprising a first heavy chain variable region (VHa) region and the first light chain variable region (VLa) that can bind a CD3 antigen, where the first heavy chain variable region (VHa) comprises a wild type Hum291 sequence (e.g., SEQ ID NO:38) or comprises at least one mutation comprising M48I/K67R, I30V/K67R, M48I, I30V/M48I/K67R, K67R, or I30V (e.g., SEQ ID NOS:22, 28, 30, 32, 34, 36, respectively). In some embodiments, the at least one mutation modulates (e.g. increases or decreases) thermal stability of the first polypeptide chain.

In one embodiment, the heterodimeric antibody comprises a first heavy chain variable region (VHa) of the first polypeptide which comprises an amino acid sequence that is at least 95% identical, at least 96% identical, at least 97% identical, at least 98% identical, at least 99% identical, or 100% identical, to one of the amino acid sequences selected from SEQ ID NOS:22, 28, 30, 32, 34, 36 and 38.

In one embodiment, the heterodimeric antibody comprises a first light chain variable region (VLa) of the first polypeptide which comprises an amino acid sequence that is at least 95% identical, at least 96% identical, at least 97% identical, at least 98% identical, at least 99% identical, or 100% identical, to one of the amino acid sequences selected from SEQ ID NOS:24, 29, 31, 33, 35, 37 and 39.

In one embodiment, the heterodimeric antibody comprises a first polypeptide chain which comprises an amino acid sequence that is at least 95% identical, at least 96% identical, at least 97% identical, at least 98% identical, at least 99% identical, or 100% identical, to one of the amino acid sequences selected from SEQ ID NOS:71, 72, 73, 74, 75, 76 and 77.

In one embodiment, the heterodimeric antibody comprises a first polypeptide comprising the amino acid sequence of SEQ ID NO:87 (e.g., BZ1).

In one embodiment, the heterodimeric antibody comprises a first polypeptide comprising the amino acid sequence of SEQ ID NO:90 (e.g., BZ1S).

The present disclosure provides a heterodimeric antibody comprising a second polypeptide chain comprising a second heavy chain variable region (VHb) which comprises humanized or a fully human immunoglobulin sequences.

In one embodiment, the heterodimeric antibody comprises a second heavy chain variable region (VHb) of the second polypeptide which comprises an amino acid sequence that is at least 95% identical, at least 96% identical, at least 97% identical, at least 98% identical, at least 99% identical, or 100% identical, to one of the amino acid sequences selected from SEQ ID NOS:1, 6, 8, 10, 12, 14, 19, 40, 42, 44, 46, 48, 50, 52, 54, 56, 58, 60, 62 and 64.

In one embodiment, the heterodimeric antibody comprises a second heavy chain constant region (CH1) of the second polypeptide which comprises an amino acid sequence that is at least 95% identical, at least 96% identical, at least 97% identical, at least 98% identical, at least 99% identical, or 100% identical, to one of the amino acid sequences selected from SEQ ID NOS: 2, 7, 9, 11, 13 or 15.

In one embodiment, the heterodimeric antibody comprises a second polypeptide chain which comprises an amino acid sequence that is at least 95% identical, at least 96% identical, at least 97% identical, at least 98% identical, at least 99% identical, or 100% identical, to one of the amino acid sequences selected from SEQ ID NOS:78, 79, 80, 81, 82 and 83.

In one embodiment, the heterodimeric antibody comprises a second polypeptide comprising the amino acid sequence of SEQ ID NO:89 (e.g., BZ1).

In one embodiment, the heterodimeric antibody comprises a second polypeptide comprising the amino acid sequence of SEQ ID NO:92 (e.g., BZ1S).

The present disclosure provides a heterodimeric antibody comprising a third polypeptide chain comprising a second light chain variable region (VLb) which comprises humanized or fully human immunoglobulin sequences.

In one embodiment, the heterodimeric antibody comprises a second light chain variable region (VLb) of the third polypeptide which comprises an amino acid sequence that is at least 95% identical, at least 96% identical, at least 97% identical, at least 98% identical, at least 99% identical, or 100% identical, to one of the amino acid sequences selected from SEQ ID NOS:16, 18, 20, 41, 43, 45, 47, 49, 51, 53, 55, 57, 59, 61, 63 and 65.

In one embodiment, the heterodimeric antibody comprises a light chain constant region (CL) of the third polypeptide which comprises an amino acid sequence that is at least 95% identical, at least 96% identical, at least 97% identical, at least 98% identical, at least 99% identical, or 100% identical, to one of the amino acid sequences selected from SEQ ID NOS:17, 19 and 21.

In one embodiment, the heterodimeric antibody comprises a third polypeptide chain which comprises an amino acid sequence that is at least 95% identical, at least 96% identical, at least 97% identical, at least 98% identical, at least 99% identical, or 100% identical, to one of the amino acid sequences selected from SEQ ID NOS:84, 85 and 86.

In one embodiment, the heterodimeric antibody comprises a third polypeptide comprising the amino acid sequence of SEQ ID NO:88 (e.g., BZ1).

In one embodiment, the heterodimeric antibody comprises a third polypeptide comprising the amino acid sequence of SEQ ID NO:91 (e.g., BZ1S).

In one embodiment, the heterodimeric antibody comprises three polypeptide chains, comprising a first polypeptide comprising the amino acid sequence of SEQ ID NO:87, and a second polypeptide comprising the amino acid sequence of SEQ ID NO:89, and a third polypeptide comprising the amino acid sequence of SEQ ID NO:88 (e.g., BZ1).

In one embodiment, the heterodimeric antibody comprises three polypeptide chains, comprising a first polypeptide comprising the amino acid sequence of SEQ ID NO:90, and a second polypeptide comprising the amino acid sequence of SEQ ID NO:92, and a third polypeptide comprising the amino acid sequence of SEQ ID NO:91 (e.g., BZ1S).

In one embodiment, the heterodimeric antibody comprises three polypeptide chains, comprising a first polypeptide comprising the amino acid sequence of any one of SEQ ID NOs:71-77, and a second polypeptide comprising the amino acid sequence of SEQ ID NO:79, and a third polypeptide comprising the amino acid sequence of SEQ ID NO:85 (e.g., CD38 A2 series).

In one embodiment, the heterodimeric antibody comprises three polypeptide chains, comprising a first polypeptide comprising the amino acid sequence of any one of SEQ ID NOs:71-77, and a second polypeptide comprising the amino acid sequence of SEQ ID NO:78, and a third polypeptide comprising the amino acid sequence of SEQ ID NO:84 (e.g., CD38 A2-3H10m1 series).

In one embodiment, the heterodimeric antibody comprises three polypeptide chains, comprising a first polypeptide comprising the amino acid sequence of any one of SEQ ID NOs:71-77, and a second polypeptide comprising the amino acid sequence of any one of SEQ ID NOS:80-83, and a third polypeptide comprising the amino acid sequence of SEQ ID NO:86 (e.g., CD38 D8 series).

In one embodiment, the heterodimeric antibody comprises three polypeptide chains, comprising a first polypeptide comprising the amino acid sequence of SEQ ID NO:100, and a second polypeptide comprising the amino acid sequence of SEQ ID NO:106, and a third polypeptide comprising the amino acid sequence of SEQ ID NO:86 (e.g., CD38 D8 bump-in-dent series).

The present disclosure provides a heterodimeric antibody comprising a peptide linker of the first polypeptide which comprises a peptide linker which can be a flexible peptide linker.

In one embodiment, the peptide comprises an amino acid sequence selected from (SG)ₙ, (SGG)ₙ, (SGGG)ₙ (SEQ ID NO: 112), (SSG)ₙ, (GS)ₙ, (GGG)ₙ, (GSGGS)ₙ (SEQ ID NO: 113), (GSG)ₙ, (GGGGS)ₙ (SEQ ID NO: 114), (GGGS)ₙ (SEQ ID NO: 115), (GGGGSGS)ₙ (SEQ ID NO: 116), (GGGGSGGS)ₙ (SEQ ID NO: 117), and (GGS)ₙ, where n is an integer of 1-6.

In one embodiment, the peptide linker comprises an amino acid sequence of TSGSGGSGGSV (SEQ ID NO: 118). In one embodiment, the peptide linker of the first polypeptide comprises the amino acid sequence of SEQ ID NO:23.

The present disclosure provides a heterodimeric antibody comprising a first hinge region of the first polypeptide which comprises an IgG1, IgG2, IgG3 or IgG4 hinge region.

In one embodiment, the first hinge region of the first polypeptide comprises the amino acid sequence of SEQ ID NO:25. In one embodiment, the first hinge region having the amino acid sequence of SEQ ID NO:25 is mutated so that CPPC is mutated to SPPC.

The present disclosure provides a heterodimeric antibody comprising a second hinge region of the second polypeptide which comprises an IgG1, IgG2, IgG3 or IgG4 hinge region.

In one embodiment, the second hinge region of the second polypeptide comprises the amino acid sequence of SEQ ID NO:3.

The present disclosure provides a heterodimeric antibody comprising a first Fc region of the first polypeptide and the second Fc region of the second polypeptide which form an Fc domain that can bind Fc cell surface receptors.

The present disclosure provides a heterodimeric antibody comprising a first Fc region of the first polypeptide and the second Fc region of the second polypeptide which form an Fc domain that exhibits effector function including complement-dependent cytotoxicity (CDC), antibody-dependent cell-mediated cytotoxicity (ADCC), antibody-dependent phagocytosis (ADP), opsonization and/or cell binding.

In one embodiment, the first Fc region of the first polypeptide and/or the second Fc region of the second polypeptide include a mutation that reduces Fc effector function.

In one embodiment, the first Fc region of the first polypeptide and/or the second Fc region of the second polypeptide include a LALA mutation.

In one embodiment, the first Fc region of the first polypeptide and/or the second Fc region of the second polypeptide are mutated to form knob-in-hole structures. In one embodiment, the first Fc region of the first polypeptide comprises the amino acid sequences of SEQ ID NOS:26 and 27. In one embodiment, the second Fc region of the second polypeptide comprises the amino acid sequences of SEQ ID NOS:4 and 5.

In one embodiment, the first Fc region of the first polypeptide and/or the second Fc region of the second polypeptide are mutated to form bump-in-dent structures. In one embodiment, the first Fc region of the first polypeptide comprises the amino acid sequences of SEQ ID NOS:98 and 99. In one embodiment, the second Fc region of the second polypeptide comprises the amino acid sequences of SEQ ID NOS:104 and 105.

The present disclosure provides one or more nucleic acids encoding the polypeptides of a heterodimeric antibody described herein. In some embodiments, the one or more nucleic acids comprise a first nucleic acid encoding any of the full-length first polypeptides described herein or any portions thereof.

In one embodiment, the first nucleic acid encodes the first heavy chain variable region (VHa) having the amino acid sequence selected from SEQ ID NOS:22, 28, 30, 32, 34, 36 and 38.

In one embodiment, the first nucleic acid encodes the peptide linker having an amino acid sequence selected from (SG)ₙ, (SGG)ₙ, (SGGG)ₙ (SEQ ID NO: 112), (SSG)ₙ, (GS)ₙ, (GGG)ₙ, (GSGGS)ₙ (SEQ ID NO: 113), (GSG)ₙ, (GGGGS)ₙ (SEQ ID NO: 114), (GGGS)ₙ (SEQ ID NO: 115), (GGGGSGS)ₙ (SEQ ID NO: 116), (GGGGSGGS)ₙ (SEQ ID NO: 117), and (GGS)ₙ, where n is an integer of 1-6. In one embodiment, the first nucleic acid encodes the peptide linker having an amino acid sequence of TSGSGGSGGSV (SEQ ID NO: 118). In one embodiment, the first nucleic acid encodes the peptide linker having the amino acid sequence of SEQ ID NO:23.

In one embodiment, the first nucleic acid encodes the first light chain variable region (VLa) having the amino acid sequence selected from SEQ ID NOS:24, 29, 31, 33, 35, 37 and 39.

In one embodiment, the first nucleic acid encodes the first hinge region having the amino acid sequence of SEQ ID NO:25. In one embodiment, the first nucleic acid encodes the first hinge region having the amino acid sequence of SEQ ID NO:25 is mutated so that CPPC is mutated to SPPC.

In one embodiment, the first nucleic acid encodes the CH2 region of the first polypeptide having the amino acid sequence of SEQ ID NO:26.

In one embodiment, the first nucleic acid encodes the CH3 region of the first polypeptide having the amino acid sequence of SEQ ID NO:27.

In one embodiment, the first nucleic acid encodes a full-length first polypeptide having the amino acid sequence of SEQ ID NO:71, 72, 73, 74, 75, 76 or 77.

In some embodiments, the one or more nucleic acids comprise a second nucleic acid encoding any of the full-length second polypeptides described herein or any portions thereof.

In one embodiment, the second nucleic acid encodes the second heavy chain variable region (VHb) of the second polypeptide having the amino acid sequence selected from SEQ ID NOS: 1, 6, 8, 10, 12, 14, 40, 42, 44, 46, 48, 50, 52, 54, 56, 58, 60, 62 and 64.

In one embodiment, the second nucleic acid encodes the heavy chain constant region (CH1) of the second polypeptide having the amino acid sequence selected from SEQ ID NO:2, 7, 9, 11, 13 and 15.

In one embodiment, the second nucleic acid encodes the second hinge region of the second polypeptide having the amino acid sequence of SEQ ID NO:3. In one embodiment, the second nucleic acid encodes the second hinge region having the amino acid sequence of SEQ ID NO:3 is mutated so that CPPC is mutated to SPPC.

In one embodiment, the second nucleic acid encodes the CH2 region of the second polypeptide having the amino acid sequence of SEQ ID NO:4.

In one embodiment, the second nucleic acid encodes the CH3 region of the second polypeptide having the amino acid sequence of SEQ ID NO:5.

In one embodiment, the second nucleic acid encodes a full-length second polypeptide having the amino acid sequence of SEQ ID NO:78, 79, 80, 81, 82 or 83.

In some embodiments, the one or more nucleic acids comprise a third nucleic acid encoding any of the full-length third polypeptides described herein or any portions thereof.

In one embodiment, the third nucleic acid encodes the second light chain variable region (VLb) of the third polypeptide having the amino acid sequence selected from SEQ ID NOS:16, 18, 20, 41, 43, 45, 47, 49, 51, 53, 55, 57, 59, 61, 63 and 65.

In one embodiment, the third nucleic acid encodes the light chain constant region (CL1) of the third polypeptide having the amino acid sequence selected from SEQ ID NO:17, 19 and 21.

In one embodiment, the third nucleic acid encodes a full-length third polypeptide having the amino acid sequence of SEQ ID NO:84, 85 or 86.

The present disclosure provides one or more nucleic acids encoding a first polypeptide having the amino acid sequence of SEQ ID NO:87, a second polypeptide having the amino acid sequence of SEQ ID NO:89, and a third nucleic acid encoding a third polypeptide having the amino acid sequence of SEQ ID NO:88.

The present disclosure provides a first nucleic acid encoding a first polypeptide having the amino acid sequence of SEQ ID NO:87.

The present disclosure provides a second nucleic acid encoding a second polypeptide having the amino acid sequence of SEQ ID NO:89.

The present disclosure provides a third nucleic acid encoding a third polypeptide having the amino acid sequence of SEQ ID NO:88.

The present disclosure provides one or more nucleic acids encoding a first polypeptide having the amino acid sequence of SEQ ID NO:90, a second polypeptide having the amino acid sequence of SEQ ID NO:92, and a third nucleic acid encoding a third polypeptide having the amino acid sequence of SEQ ID NO:91.

The present disclosure provides a first nucleic acid encoding a first polypeptide having the amino acid sequence of SEQ ID NO:90.

The present disclosure provides a second nucleic acid encoding a second polypeptide having the amino acid sequence of SEQ ID NO:92.

The present disclosure provides a third nucleic acid encoding a third polypeptide having the amino acid sequence of SEQ ID NO:91.

The present disclosure provides individual vectors, including expression vectors, that are operably joined to one or more nucleic acids (e.g., nucleic acid transgene(s)) that encode any of the first, second and/or third polypeptide that make up any of the heterodimeric antibodies described herein. In one embodiment, the expression vector comprises one or more promoters which control transcription of the nucleic acid encoding the first, second or third polypeptide.

In one embodiment, the vector comprises at least one regulatory sequence, for example a promoter and optionally an enhancer, that is operably joined to a nucleic acid that encodes the first, second or third polypeptide, wherein the promoter controls transcription of the nucleic acid encoding the first, second or third polypeptide in a mono-cistronic manner.

In one embodiment, the vector comprises a promoter (and optionally an enhancer) that is operably joined to any two or any combination of multiple nucleic acids that encode the first, second and/or third polypeptide, where the promoter controls transcription of a polycistronic transcript encoding the first, second and/or third polypeptides.

In one embodiment, the vector comprises multiple promoters (and optionally at least one enhancer sequence) to permit operably joining individual promoters to individual nucleic acids each encoding the first, second or third polypeptide, wherein multiple promoters within a single vector control transcription of different transcript encoding the first, second and/or third polypeptides.

In one embodiment, one vector is introduced into a host cell, wherein the vector within the host cell carries a promoter (and optionally an enhancer sequence), and one nucleic acid that encodes a polypeptide (e.g., first, second or third polypeptide) is joined to the promoter in the vector. Thus, the host cell can express the first, second or third polypeptide that make up any of the heterodimeric antibodies.

In one embodiment, multiple vectors are introduced into a host cell, wherein individual vectors within a host cell carry at least one promoter (and optionally an enhancer sequence), and one nucleic acid that encodes a polypeptide (e.g., first, second or third polypeptide) is joined to one promoter in one vector. Thus, individual host cells can express any two or any combination of the first, second and/or third polypeptides that make up any of the heterodimeric antibodies.

The vectors comprise promoters that are inducible or constitutive promoters. The vectors and host cells can be selected to generate transgenic host cells that transiently or stably express any of the polypeptide described herein.

In one embodiment, a first expression vector is operably joined to a first nucleic acid encoding any of the full-length first polypeptides described herein or any portions thereof.

In one embodiment, a second expression vector is operably joined to a second nucleic acid encoding any of the full-length second polypeptides described herein or any portions thereof.

In one embodiment, a third expression vector is operably joined to a third nucleic acid encoding any of the full-length third polypeptides described herein or any portions thereof.

In one embodiment, an expression vector system includes two different expression vectors. In one embodiment, the expression vector system comprises a first expression vector that is operably joined to two nucleic acids including a first nucleic acid encoding any of the full-length first polypeptides described herein or any portions thereof, and the first expression vector is operably joined to a second nucleic acid encoding any of the full-length second polypeptides described herein or any portions thereof. In one embodiment, the expression vector system comprises a second expression vector that is operably joined to a third nucleic acid encoding any of the full-length third polypeptides described herein or any portions thereof.

In one embodiment, an expression vector system includes two different expression vectors. In one embodiment, the expression vector system comprises a first expression vector that is operably joined to two nucleic acids including a second nucleic acid encoding any of the full-length second polypeptides described herein or any portions thereof, and the first expression vector is operably joined to a third nucleic acid encoding any of the full-length third polypeptides described herein or any portions thereof. In one embodiment, the expression vector system comprises a second expression vector that is operably joined to a first nucleic acid encoding any of the full-length first polypeptides described herein or any portions thereof.

In one embodiment, an expression vector system includes two different expression vectors. In one embodiment, the expression vector system comprises a first expression vector that is operably joined to two nucleic acids including a first nucleic acid encoding any of the full-length first polypeptides described herein or any portions thereof, and the first expression vector is operably joined to a third nucleic acid encoding any of the full-length third polypeptides described herein or any portions thereof. In one embodiment, the expression vector system comprises a second expression vector that is operably joined to a second nucleic acid encoding any of the full-length second polypeptides described herein or any portions thereof.

In one embodiment, an expression vector system includes a single expression vector that is operably joined to three nucleic acids including a first nucleic acid encoding any of the full-length first polypeptides described herein or any portions thereof, and the single expression vector is operably joined to a second nucleic acid encoding any of the full-length second polypeptides described herein or any portions thereof, and the single expression vector is operably joined to a third nucleic acid encoding any of the full-length third polypeptides described herein or any portions thereof.

In one embodiment, any of the first and/or second expression vectors, or the single expression vector, comprises a nucleic acid sequence encoding glutamine synthetase.

The present disclosure provides host cells that harbor one or more vectors that encode any, or each, of the first, second and/or third polypeptide that make up any of the heterodimeric antibodies described herein. Here and throughout, “encode” in the context of a vector means that the vector contains sequence(s) encoding the recited polypeptide(s) operably linked to promoter(s) such that the vector can be used to express the polypeptide(s).

The present disclosure provides host cells that harbor a single vector that is operably joined to one or more nucleic acids that encode any of the first, second and/or third polypeptide that make up any of the heterodimeric antibodies.

The present disclosure provides host cells that harbor two or more vectors each vector being operably joined to one or more nucleic acids that encode the first, second and/or third polypeptide that make up any of the heterodimeric antibodies.

The host cell can be a bacterial or mammalian cell. In one embodiment, the host cell comprises a Chinese hamster ovary (CHO) cell.

In one embodiment, at least one vector is introduced into the host cell via lipofection (e.g., using a lipid surfactant); electroporation; transfection employing calcium chloride, rubidium chloride, calcium phosphate, DEAE-dextran, or other substances; viral transfection; non-viral transfection; microprojectile bombardment; and infection (e.g., where the vector is an infectious agent).

In one embodiment, host cells harbor two vectors that are present in the host cell at a molar ratio of 1:1, 1:1.5, 1.5:1, 1:2, 2:1, 1:3, or 3:1. Other molar ratios are possible as well known in the art.

The present disclosure provides methods for preparing any of the heterodimeric antibodies described herein, the method comprising: culturing a population of host cells comprising one or more nucleic acids encoding a heterodimeric antibody described herein, wherein the culturing is conducted under conditions suitable for expressing the polypeptides of the heterodimeric antibody by the population of host cells. The present disclosure provides methods for preparing any of the heterodimeric antibodies described herein, the method comprising: culturing a population of host cells wherein individual host cells in the population harbor at least one expression vector that is operably linked to any one or any combination of two or more first, second and/or third nucleic acids encoding any one or any combination of two or more of the first, second and/or third polypeptides described herein) wherein the culturing is conducted under conditions suitable for expressing the polypeptide by the population of host cells.

In one embodiment, the nucleic acids encoding any one or any combination of two or more of the first, second and/or third polypeptides further encodes a signal peptide for secretion of the expressed polypeptides. In one embodiment, the culturing is conducted under conditions suitable for secretion of the first, second and/or third polypeptides by the population of host cells. Exemplary signal peptides comprise the amino acid sequence MEWSWVFLFFLSVTTGVHS (SEQ ID NO: 111) or MSVPTQVLGLLLLWLTDARC (SEQ ID NO: 119).

In one embodiment, the nucleic acids encoding any one or any combination of two or more of the first, second and/or third polypeptides further encodes an affinity tag sequence for enriching the polypeptides. Exemplary affinity tag sequences include histidine tag, FLAG tag, myc tag, HA tag, and GST tag.

In one embodiment, the method further comprises isolating the expressed first, second and/or third polypeptide.

In one embodiment, the culturing is conducted under conditions that are suitable for assembly or association of the first, second and/or third polypeptide to form the heterodimeric antibodies. In one embodiment, the first polypeptide folds to form an scFv-Fc fusion polypeptide that can bind a CD3 antigen. In one embodiment, the second and third polypeptides associate with each other to form a half immunoglobulin molecule having an antigen binding domain that can bind a CD38 antigen. In one embodiment, the first, second and third polypeptides associate with each other form the heterodimeric antibody (e.g., a bispecific antibody) that can bind CD3 and CD38 antigens.

In one embodiment, the method further comprises isolating or recovering the associated/assembled heterodimeric antibodies. In one embodiment, the isolating is conducted using affinity chromatography. In one embodiment, the isolating is conducted using affinity chromatography with protein A or G from Staphylococcus aureus, glutathione S-transferase (GST), or immuno-affinity. In one embodiment, one or more additional isolating steps are conducted which are selected from cation and/or anion exchange chromatography, hydrophobic interaction chromatography, mixed mode chromatography and hydroxyapatite chromatography.

The heterodimeric antibodies can be prepared using transgenic host cell expression, phage display, yeast display and human antibody gene transgenic mice using methods that are well known in the art. In one embodiment, the yield of heterodimeric antibodies using transgenic host cell expression can be about 20-80%, or about 30-90%, or about 40-95%, or about 50-99% of the total protein complexes formed.

The present disclosure provides heterodimeric antibodies that behave as bispecific antibodies that can bind two different target antigens at the same time. For example, the heterodimeric antibodies can bind a tumor-associated antigen and an antigen expressed on an effector T cell. The heterodimeric antibodies described herein are designed to generate immune cell synapse which leads to tumor-selective cytotoxic cell killing. Additionally, when the heterodimeric antibody includes a functional Fc domain, then the Fc domain can bind an Fc receptor thereby forming a three-way immune cell synapse comprising the heterodimeric antibody binding at the same time to an effector T cell, a tumor cell expressing a target tumor antigen, and an Fc receptor (e.g., macrophage, natural killer cell or dendritic cell). The three-way immune cell synapse can mediate cytotoxic cell killing.

The present disclosure provide methods for binding any of the heterodimeric antibodies described herein to first and second target antigens, the method comprising: contacting the first target antigen (e.g., CD38 antigen) and second target antigen (e.g., CD3 antigen) with a heterodimeric antibody which comprises a first polypeptide that forms an scFv-Fc fusion polypeptide that binds a first target antigen and the heterodimeric antibody comprises a second and third polypeptide that associate to form a half immunoglobulin molecule that binds a second target antigen (e.g., CD38 antigen).

In one embodiment, the heterodimeric antibody can be contacted with the first and second target antigens at the same time. In one embodiment, the heterodimeric antibody can be contacted with the first and second target antigens sequentially, in any order. In one embodiment, the heterodimeric antibody can bind the first and second target antigens simultaneously.

In one embodiment, the method further comprises: forming a cell synapse by binding the heterodimeric antibody with the first target antigen (e.g., expressed by the tumor or cancer cell) and with the second target antigen (e.g., expressed by the effector T cell) so that the tumor cell and effector T cell are in close proximity to each other.

In one embodiment, method further comprises: killing the tumor or cancer cell with the effector T cell in the cell synapse which mediates cytotoxic cell killing.

The present disclosure provide methods treating a subject having a disease associated with expression or over-expression of a tumor-associated antigen, the method comprising: administering to the subject a therapeutically effective amount of a composition which comprises a heterodimeric antibody that can bind two different target antigens. Disclosure of a method of treatment in which a composition or heterodimeric antibody is administered also constitutes (1) disclosure of the use of the composition or heterodimeric antibody for the manufacture of a medicament for such treatment and (2) disclosure of the composition or heterodimeric antibody for use in such treatment.

In one embodiment, the subject has a disease comprising cancer of an organ selected from prostate, breast, ovary, head and neck, bladder, skin, colorectal, anus, rectum, pancreas, lung (including non-small cell lung and small cell lung cancers), brain, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis, and testis, or a leiomyoma, glioma, or glioblastoma.

In one embodiment, the subject has a disease comprising a hematologic cancer. In some embodiments, the hematologic cancer is B chronic lymphocytic leukemia (B-CLL), B and T acute lymphocytic leukemia (LL), acute myeloid leukemia (AML), chronic lymphocytic leukemia (CLL), chronic myelogenous leukemia (CML), hairy cell leukemia (HCL), myeloproliferative disorder/neoplasm (MPDS), myelodysplasia syndrome, non-Hodgkin's lymphoma (NHL) including Burkitt's lymphoma (BL), Waldenstrom's Macroglobulinemia, mantle cell lymphoma, AIDS-related lymphoma, Hodgkin's Lymphoma (HL), T cell lymphoma (TCL), multiple myeloma (MM), plasma cell myeloma, plamocytoma, giant cell myeloma, heavy-chain myeloma, or light chain or Bence-Jones myeloma.

The present disclosure provides any heterodimeric antibody described herein, wherein the first or second light chain variable regions comprise amino acid sequence from κ or λ chains.

The present disclosure provides any heterodimeric antibody described herein, wherein the first or second heavy chain variable regions comprise amino acid sequences from μ, γ, α, δ or ε chains.

The present disclosure provides any heterodimeric antibody described herein, wherein a variable heavy region and a constant heavy region on the second polypeptide is directly joined together without any intervening linker sequences which avoids introducing immunogenic sites.

The present disclosure provides any heterodimeric antibody described herein, wherein the variable light region and constant light region on the third polypeptide is directly joined together without any intervening linker sequences which avoids introducing immunogenic sites.

The present disclosure provides any heterodimeric antibody described herein, wherein the first antigen binding domain formed by the VHa and VLa regions of the first polypeptide can bind the first target antigen expressed by a cell, wherein the cell is selected from a cell surface antigen on a T cell (e.g., effector T cell), a NK cell, a monocyte, a neutrophil or a macrophage.

In one embodiment, the first antigen binding domain formed by the VHa and VLa regions of the first polypeptide can bind the first target antigen and exhibit a dissociation constant K_{d} of 10⁻⁵ M or less, or 10⁻⁶ M or less, or 10⁻⁷ M or less, or 10⁻⁸ M or less, or 10⁻⁹ M or less, or 10⁻¹⁰ M or less.

The present disclosure provides any heterodimeric antibody described herein, wherein the second antigen binding domain formed by the second and third polypeptides can bind a second target antigen comprising a tumor-associated antigen expressed by a cancer cell wherein the cancer cell is selected from cancer of the prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis and testis.

In one embodiment, the second antigen binding domain is capable of binding the second target antigen and exhibit a dissociation constant K_{d} of 10⁻⁵ M or less, or 10⁻⁶ M or less, or 10⁻⁷ M or less, or 10⁻⁸ M or less, or 10⁻⁹ M or less, or 10⁻¹⁰ M or less.

The present disclosure provides any heterodimeric antibody described herein, wherein the Fc domain comprises CH2 and CH3 sequences in the first polypeptide. In one embodiment, the CH2 and CH3 are directly joined together without any intervening amino acids or peptide linker sequence. In one embodiment, the Fc domain include a hinge sequence joined to the amino-terminus of the CH2 region. In one embodiment, the N-terminus of the hinge sequence includes a hinge linker sequence GGSGG (SEQ ID NO:110). In one embodiment, the heterodimeric antibody comprises an Fc domain which exhibits effector function, or exhibits reduced effector function, where the effector function includes complement-dependent cytotoxicity (CDC), antibody-dependent cell-mediated cytotoxicity (ADCC) and/or antibody-dependent phagocytosis (ADP). In one embodiment, the hinge region includes a mutation that reduces Fc effector function, for example a LALA mutation (L234A, L235A) or a LALA-PG mutation (L234A, L235A, P329G). The amino acid numbering is based on Kabat numbering. In one embodiment, the carboxy-terminus of the hinge region is joined to the amino-terminus of the CH2 region without any intervening amino acids or peptide linker sequence. In one embodiment, the Fc domain mediates serum half-life of the protein complex, and a mutation in the Fc domain can increase or decrease the serum half-life of the protein complex. For example, the Fc domain comprises a YTE mutation (e.g., M252Y, S254T, T256E) according to Kabat numbering. In one embodiment, the Fc domain affects thermal stability of the protein complex, and mutation in the Fc domain can increase or decrease the thermal stability of the protein complex. In one embodiment, the Fc domain affects thermal stability of the protein complex, and mutation in the Fc domain can increase or decrease the thermal stability of the protein complex.

The present disclosure provides any heterodimeric antibody described herein, wherein the Fc domain comprises CH2 and CH3 sequences on the second polypeptide. In one embodiment, the CH2 and CH3 are directly joined together without any intervening amino acids or peptide linker sequence. In one embodiment, the Fc domain include a hinge sequence joined to the amino-terminus of the CH2 region. In one embodiment, the N-terminus of the hinge sequence lacks a hinge linker sequence GGSGG (SEQ ID NO:110). In one embodiment, the heterodimeric antibody comprises an Fc domain which exhibits effector function, or exhibits reduced effector function, where the effector function includes complement-dependent cytotoxicity (CDC), antibody-dependent cell-mediated cytotoxicity (ADCC) and/or antibody-dependent phagocytosis (ADP). In one embodiment, the hinge region includes a mutation that reduces Fc effector function, for example a LALA mutation (L234A, L235A) or a LALA-PG mutation (L234A, L235A, P329G). The amino acid numbering is based on Kabat numbering. In one embodiment, the carboxy-terminus of the hinge region is joined to the amino-terminus of the CH2 region without any intervening amino acids or peptide linker sequence. In one embodiment, the Fc domain mediates serum half-life of the protein complex, and a mutation in the Fc domain can increase or decrease the serum half-life of the protein complex. For example, the Fc domain comprises a YTE mutation (e.g., M252Y, S254T, T256E) according to Kabat numbering. In one embodiment, the Fc domain affects thermal stability of the protein complex, and mutation in the Fc domain can increase or decrease the thermal stability of the protein complex. In one embodiment, the Fc domain affects thermal stability of the protein complex, and mutation in the Fc domain can increase or decrease the thermal stability of the protein complex.

The present disclosure provides any heterodimeric antibody described herein, comprising one or more amino acid mutations in the CH2 and/or CH3 region of the first polypeptide, and comprising one or more amino acid mutations in the CH2 and/or CH3 region of the second polypeptide, that promotes formation of heterodimers in an assembled protein complex, where the mutations includes introducing knob-in-hole structures (Ridgeway 1996 Protein Engineering 9(7):617-621), introducing additional interchain disulfide bonding (Carter 2011 Journal of Immunological Methods 248:7-15), and/or introducing new salt bridges.

In one embodiment, one of the polypeptide chains (e.g., the first or second polypeptide) is mutated in the Fc region (e.g., CH3 region) by substituting a small amino acid with a larger one to create a protrusion (e.g., a knob or bump). In one embodiment, another polypeptide chain (e.g., the second or first polypeptide) is mutated in the Fc region (e.g., CH3 region) by substituting a larger amino acid with a smaller one to create a socket (e.g., a hole or dent). In one embodiment, Fc domain knob-in-hole mutations comprise a substitute mutation at any one Fc location or any combination of two or more Fc locations selected from T366, L368, T394, F405, Y407 and K409 (numbering is based on Kabat system). In one embodiment, Fc domain knob-in-hole mutation comprises any one or any combination of two or more of the following mutations: T366Y, T366W, T366S, L368A, T394S, T394W, F405A, F405W, Y407A, Y407V, Y407T (numbering based on Kabat system). In one embodiment, the Fc domain bump-in-dent mutation comprises F405L (numbering based on Kabat system).

The present disclosure provides any heterodimeric antibody described herein, wherein the hinge region comprises any one or any combination of two or more regions comprising an upper, core or lower hinge sequences from an IgG1, IgG2, IgG3 or IgG4 immunoglobulin molecule. In one embodiment, the hinge region comprises an IgG1 upper hinge sequence EPKSCDKTHT (SEQ ID NO: 120). In one embodiment, the hinge region comprises an IgG1 core hinge sequence CPXC, wherein X is P, R or S (SEQ ID NO: 121). In one embodiment, the hinge region comprises a lower hinge/CH2 sequence PAPELLGGP (SEQ ID NO: 122). In one embodiment, the hinge is joined to an Fc region (CH2) having the amino acid sequence SVFLFPPKPKDT (SEQ ID NO: 123). In one embodiment, the hinge region includes the amino acid sequence of an upper, core and lower hinge and comprises EPKSCDKTHTCPPCPAP ELLGGP (SEQ ID NO: 124). In one embodiment, the hinge region comprises one, two, three or more cysteines that can form at least one, two, three or more interchain disulfide bonds.

### EXEMPLARY SEQUENCES

TABLE Q
FIRST POLYPEPTIDE: scFv-Fc fusion
CD3-tsm#1 (M48I/K67R)

| | |
|---|---|
| Heavy chain | EVQLVQSGAEVKKPGASVKVSCKASGYTFISYTMH |
| variable | WVRQAPGQGLEWIGYINPRSGYTHYNQKLKDRATL |
| SEQ ID NO: 22 | TADKSASTAYMELSSLRSEDTAVYYCARSAYYDYD |
| | GFAYWGQGTLVTVSS |
| Linker | GGGGSGGGGSGGGGSGGGGS |
| SEQ ID NO: 23 | |
| Light chain | DIQMTQSPSSLSASVGDRVTITCSASSSVSYMNWY |
| variable | QQKPGKAPKRLIYDTSKLASGVPSRFSGSGSGTDF |
| SEQ ID NO: 24 | TLTISSLQPEDFATYYCQQWSSNPPTFGGGTKVEI |
| | K |
| Hinge: | GGSGGDKTHTCPPCPAPEAAGGPSV |
| SEQ ID NO: 25 | |
| CH2: | FLFPPKPKDTLMISRTPEVTCVVVDVSHEDPEVKF |
| SEQ ID NO: 26 | NWYVDGVEVHNAKTKPREEQYNSTYRVVSVLTVLH |
| | QDWLNGKEYKCKVSNKALPAPIEKTISKAK |
| CH3: | GQPREPQVYTLPPSREEMTKNQVSLSCAVKGFYPS |
| SEQ ID NO: 27 | DIAVEWESNGQPENNYKTTPPVLDSDGSFFLVSKL |
| | TVDKSRWQQGNVFSCSVMHEALHNHYTQKSLSLSP |
| | GK |
| Full-length | EVQLVQSGAEVKKPGASVKVSCKASGYTFISYTMH |
| sequence: | WVRQAPGQGLEWIGYINPRSGYTHYNQKLKDRATL |
| SEQ ID NO: 71 | TADKSASTAYMELSSLRSEDTAVYYCARSAYYDYD |
| | GFAYWGQGTLVTVSSGGGGSGGGGSGGGGSGGGGS |
| | DIQMTQSPSSLSASVGDRVTITCSASSSVSYMNWY |
| | QQKPGKAPKRLIYDTSKLASGVPSRFSGSGSGTDF |
| | TLTISSLQPEDFATYYCQQWSSNPPTFGGGTKVEI |
| | KGGSGGDKTHTCPPCPAPEAAGGPSVFLFPPKPKD |
| | TLMISRTPEVTCVVVDVSHEDPEVKFNWYVDGVEV |
| | HNAKTKPREEQYNSTYRVVSVLTVLHQDWLNGKEY |
| | KCKVSNKALPAPIEKTISKAKGQPREPQVYTLPPS |
| | REEMTKNQVSLSCAVKGFYPSDIAVEWESNGQPEN |
| | NYKTTPPVLDSDGSFFLVSKLTVDKSRWQQGNVFS |
| | CSVMHEALHNHYTQKSLSLSPGK |

TABLE 2
FIRST POLYPEPTIDE: scFv-Fc fusion
CD3-tsm#2 (I30V/K67R

| | |
|---|---|
| Heavy chain | EVQLVQSGAEVKKPGASVKVSCKASGYTFVSYTMH |
| variable | WVRQAPGQGLEWMGYINPRSGYTHYNQKLKDRATL |
| SEQ ID NO: 28 | TADKSASTAYMELSSLRSEDTAVYYCARSAYYDYD |
| | GFAYWGQGTLVTVSS |
| Linker | GGGGSGGGGSGGGGSGGGGS |
| SEQ ID NO: 23 | |
| Light chain | DIQMTQSPSSLSASVGDRVTITCSASSSVSYMNWY |
| variable | QQKPGKAPKRLIYDTSKLASGVPSRFSGSGSGTDF |
| SEQ ID NO: 29 | TLTISSLQPEDFATYYCQQWSSNPPTFGGGTKVEI |
| | K |
| Hinge: | GGSGGDKTHTCPPCPAPEAAGGPSV |
| SEQ ID NO: 25 | |
| CH2: | FLFPPKPKDTLMISRTPEVTCVVVDVSHEDPEVKF |
| SEQ ID NO: 26 | NWYVDGVEVHNAKTKPREEQYNSTYRVVSVLTVLH |
| | QDWLNGKEYKCKVSNKALPAPIEKTISKAK |
| CH3: | GQPREPQVYTLPPSREEMTKNQVSLSCAVKGFYPS |
| SEQ ID NO: 27 | DIAVEWESNGQPENNYKTTPPVLDSDGSFFLVSKL |
| | TVDKSRWQQGNVFSCSVMHEALHNHYTQKSLSLSP |
| | GK |
| Full-length | EVQLVQSGAEVKKPGASVKVSCKASGYTFVSYTMH |
| sequence: | WVRQAPGQGLEWMGYINPRSGYTHYNQKLKDRATL |
| SEQ ID NO: 72 | TADKSASTAYMELSSLRSEDTAVYYCARSAYYDYD |
| | GFAYWGQGTLVTVSSGGGGSGGGGSGGGGSGGGGS |
| | DIQMTQSPSSLSASVGDRVTITCSASSSVSYMNWY |
| | QQKPGKAPKRLIYDTSKLASGVPSRFSGSGSGTDF |
| | TLTISSLQPEDFATYYCQQWSSNPPTFGGGTKVEI |
| | KGGSGGDKTHTCPPCPAPEAAGGPSVFLFPPKPKD |
| | TLMISRTPEVTCVVVDVSHEDPEVKFNWYVDGVEV |
| | HNAKTKPREEQYNSTYRVVSVLTVLHQDWLNGKEY |
| | KCKVSNKALPAPIEKTISKAKGQPREPQVYTLPPS |
| | REEMTKNQVSLSCAVKGFYPSDIAVEWESNGQPEN |
| | NYKTTPPVLDSDGSFFLVSKLTVDKSRWQQGNVFS |
| | CSVMHEALHNHYTQKSLSLSPGK |

TABLE 3
FIRST POLYPEPTIDE: scFv-Fc fusion
CD3-tsm#3 (M48I):

| | |
|---|---|
| Heavy chain | EVQLVQSGAEVKKPGASVKVSCKASGYTFISYTMH |
| variable | WVRQAPGQGLEWIGYINPRSGYTHYNQKLKDKATL |
| SEQ ID NO: 30 | TADKSASTAYMELSSLRSEDTAVYYCARSAYYDYD |
| | GFAYWGQGTLVTVSS |
| Linker | GGGGSGGGGSGGGGSGGGGS |
| SEQ ID NO: 23 | |
| Light chain | DIQMTQSPSSLSASVGDRVTITCSASSSVSYMNWY |
| variable | QQKPGKAPKRLIYDTSKLASGVPSRFSGSGSGTDF |
| SEQ ID NO: 31 | TLTISSLQPEDFATYYCQQWSSNPPTFGGGTKVEI |
| | K |
| Hinge: | GGSGGDKTHTCPPCPAPEAAGGPSV |
| SEQ ID NO: 25 | |
| CH2: | FLFPPKPKDTLMISRTPEVTCVVVDVSHEDPEVKF |
| SEQ ID NO: 26 | NWYVDGVEVHNAKTKPREEQYNSTYRVVSVLTVLH |
| | QDWLNGKEYKCKVSNKALPAPIEKTISKAK |
| CH3: | GQPREPQVYTLPPSREEMTKNQVSLSCAVKGFYPS |
| SEQ ID NO: 27 | DIAVEWESNGQPENNYKTTPPVLDSDGSFFLVSKL |
| | TVDKSRWQQGNVFSCSVMHEALHNHYTQKSLSLSP |
| | GK |
| Full-length | EVQLVQSGAEVKKPGASVKVSCKASGYTFISYTMH |
| sequence: | WVRQAPGQGLEWIGYINPRSGYTHYNQKLKDKATL |
| SEQ ID NO: 73 | TADKSASTAYMELSSLRSEDTAVYYCARSAYYDYD |
| | GFAYWGQGTLVTVSSGGGGSGGGGSGGGGSGGGGS |
| | DIQMTQSPSSLSASVGDRVTITCSASSSVSYMNWY |
| | QQKPGKAPKRLIYDTSKLASGVPSRFSGSGSGTDF |
| | TLTISSLQPEDFATYYCQQWSSNPPTFGGGTKVEI |
| | KGGSGGDKTHTCPPCPAPEAAGGPSVFLFPPKPKD |
| | TLMISRTPEVTCVVVDVSHEDPEVKFNWYVDGVEV |
| | HNAKTKPREEQYNSTYRVVSVLTVLHQDWLNGKEY |
| | KCKVSNKALPAPIEKTISKAKGQPREPQVYTLPPS |
| | REEMTKNQVSLSCAVKGFYPSDIAVEWESNGQPEN |
| | NYKTTPPVLDSDGSFFLVSKLTVDKSRWQQGNVFS |
| | CSVMHEALHNHYTQKSLSLSPGK |

TABLE 4
FIRST POLYPEPTIDE: scFv-Fc fusion
CD3-tsm#4 (I30V/M48I/K67R):

| | |
|---|---|
| Heavy chain | EVQLVQSGAEVKKPGASVKVSCKASGYTFVSYTMH |
| variable | WVRQAPGQGLEWIGYINPRSGYTHYNQKLKDRATL |
| SEQ ID NO: 32 | TADKSASTAYMELSSLRSEDTAVYYCARSAYYDYD |
| | GFAYWGQGTLVTVSS |
| Linker | GGGGSGGGGSGGGGSGGGGS |
| SEQ ID NO: 23 | |
| Light chain | DIQMTQSPSSLSASVGDRVTITCSASSSVSYMNWY |
| variable | QQKPGKAPKRLIYDTSKLASGVPSRFSGSGSGTDF |
| SEQ ID NO: 33 | TLTISSLQPEDFATYYCQQWSSNPPTFGGGTKVEI |
| | K |
| Hinge: | GGSGGDKTHTCPPCPAPEAAGGPSV |
| SEQ ID NO: 25 | |
| CH2: | FLFPPKPKDTLMISRTPEVTCVVVDVSHEDPEVKF |
| SEQ ID NO: 26 | NWYVDGVEVHNAKTKPREEQYNSTYRVVSVLTVLH |
| | QDWLNGKEYKCKVSNKALPAPIEKTISKAK |
| CH3: | GQPREPQVYTLPPSREEMTKNQVSLSCAVKGFYPS |
| SEQ ID NO: 27 | DIAVEWESNGQPENNYKTTPPVLDSDGSFFLVSKL |
| | TVDKSRWQQGNVFSCSVMHEALHNHYTQKSLSLSP |
| | GK |
| Full-length | EVQLVQSGAEVKKPGASVKVSCKASGYTFVSYTMH |
| sequence: | WVRQAPGQGLEWIGYINPRSGYTHYNQKLKDRATL |
| SEQ ID NO: 74 | TADKSASTAYMELSSLRSEDTAVYYCARSAYYDYD |
| | GFAYWGQGTLVTVSSGGGGSGGGGSGGGGSGGGGS |
| | DIQMTQSPSSLSASVGDRVTITCSASSSVSYMNWY |
| | QQKPGKAPKRLIYDTSKLASGVPSRFSGSGSGTDF |
| | TLTISSLQPEDFATYYCQQWSSNPPTFGGGTKVEI |
| | KGGSGGDKTHTCPPCPAPEAAGGPSVFLFPPKPKD |
| | TLMISRTPEVTCVVVDVSHEDPEVKFNWYVDGVEV |
| | HNAKTKPREEQYNSTYRVVSVLTVLHQDWLNGKEY |
| | KCKVSNKALPAPIEKTISKAKGQPREPQVYTLPPS |
| | REEMTKNQVSLSCAVKGFYPSDIAVEWESNGQPEN |
| | NYKTTPPVLDSDGSFFLVSKLTVDKSRWQQGNVFS |
| | CSVMHEALHNHYTQKSLSLSPGK |

FIRST POLYPEPTIDE: scFv-Fc fusion
CD3-tsm#5 (K67R):

| | |
|---|---|
| Heavy chain | EVQLVQSGAEVKKPGASVKVSCKASGYTFISYTMH |
| variable | WVRQAPGQGLEWMGYINPRSGYTHYNQKLKDRATL |
| SEQ ID NO: 34 | TADKSASTAYMELSSLRSEDTAVYYCARSAYYDYD |
| | GFAYWGQGTLVTVSS |
| Linker | GGGGSGGGGSGGGGSGGGGS |
| SEQ ID NO: 23 | |
| Light chain | DIQMTQSPSSLSASVGDRVTITCSASSSVSYMNWY |
| variable | QQKPGKAPKRLIYDTSKLASGVPSRFSGSGSGTDF |
| SEQ ID NO: 35 | TLTISSLQPEDFATYYCQQWSSNPPTFGGGTKVEI |
| | K |
| Hinge: | GGSGGDKTHTCPPCPAPEAAGGPSV |
| SEQ ID NO: 25 | |
| CH2: | FLFPPKPKDTLMISRTPEVTCVVVDVSHEDPEVKF |
| SEQ ID NO: 26 | NWYVDGVEVHNAKTKPREEQYNSTYRVVSVLTVLH |
| | QDWLNGKEYKCKVSNKALPAPIEKTISKAK |
| CH3: | GQPREPQVYTLPPSREEMTKNQVSLSCAVKGFYPS |
| SEQ ID NO: 27 | DIAVEWESNGQPENNYKTTPPVLDSDGSFFLVSKL |
| | TVDKSRWQQGNVFSCSVMHEALHNHYTQKSLSLSP |
| | GK |
| Full-length | EVQLVQSGAEVKKPGASVKVSCKASGYTFISYTMH |
| sequence: | WVRQAPGQGLEWMGYINPRSGYTHYNQKLKDRATL |
| SEQ ID NO: 75 | TADKSASTAYMELSSLRSEDTAVYYCARSAYYDYD |
| | GFAYWGQGTLVTVSSGGGGSGGGGSGGGGSGGGGS |
| | DIQMTQSPSSLSASVGDRVTITCSASSSVSYMNWY |
| | QQKPGKAPKRLIYDTSKLASGVPSRFSGSGSGTDF |
| | TLTISSLQPEDFATYYCQQWSSNPPTFGGGTKVEI |
| | KGGSGGDKTHTCPPCPAPEAAGGPSVFLFPPKPKD |
| | TLMISRTPEVTCVVVDVSHEDPEVKFNWYVDGVEV |
| | HNAKTKPREEQYNSTYRVVSVLTVLHQDWLNGKEY |
| | KCKVSNKALPAPIEKTISKAKGQPREPQVYTLPPS |
| | REEMTKNQVSLSCAVKGFYPSDIAVEWESNGQPEN |
| | NYKTTPPVLDSDGSFFLVSKLTVDKSRWQQGNVFS |
| | CSVMHEALHNHYTQKSLSLSPGK |

TABLE 6
FIRST POLYPEPTIDE: scFv-Fc fusion
CD3-tsm#6 ((I30V):

| | |
|---|---|
| Heavy chain | EVQLVQSGAEVKKPGASVKVSCKASGYTFVSYTMH |
| variable | WVRQAPGQGLEWMGYINPRSGYTHYNQKLKDKATL |
| SEQ ID NO: 36 | TADKSASTAYMELSSLRSEDTAVYYCARSAYYDYD |
| | GFAYWGQGTLVTVSS |
| Linker | GGGGSGGGGSGGGGSGGGGS |
| SEQ ID NO: 23 | |
| Light chain | DIQMTQSPSSLSASVGDRVTITCSASSSVSYMNWY |
| variable | QQKPGKAPKRLIYDTSKLASGVPSRFSGSGSGTDF |
| SEQ ID NO: 37 | TLTISSLQPEDFATYYCQQWSSNPPTFGGGTKVEI |
| | K |
| Hinge: | GGSGGDKTHTCPPCPAPEAAGGPSV |
| SEQ ID NO: 25 | |
| CH2: | FLFPPKPKDTLMISRTPEVTCVVVDVSHEDPEVKF |
| SEQ ID NO: 26 | NWYVDGVEVHNAKTKPREEQYNSTYRVVSVLTVLH |
| | QDWLNGKEYKCKVSNKALPAPIEKTISKAK |
| CH3: | GQPREPQVYTLPPSREEMTKNQVSLSCAVKGFYPS |
| SEQ ID NO: 27 | DIAVEWESNGQPENNYKTTPPVLDSDGSFFLVSKL |
| | TVDKSRWQQGNVFSCSVMHEALHNHYTQKSLSLSP |
| | GK |
| Full-length | EVQLVQSGAEVKKPGASVKVSCKASGYTFVSYTMH |
| sequence: | WVRQAPGQGLEWMGYINPRSGYTHYNQKLKDKATL |
| SEQ ID NO: 76 | TADKSASTAYMELSSLRSEDTAVYYCARSAYYDYD |
| | GFAYWGQGTLVTVSSGGGGSGGGGSGGGGSGGGGS |
| | DIQMTQSPSSLSASVGDRVTITCSASSSVSYMNWY |
| | QQKPGKAPKRLIYDTSKLASGVPSRFSGSGSGTDF |
| | TLTISSLQPEDFATYYCQQWSSNPPTFGGGTKVEI |
| | KGGSGGDKTHTCPPCPAPEAAGGPSVFLFPPKPKD |
| | TLMISRTPEVTCVVVDVSHEDPEVKFNWYVDGVEV |
| | HNAKTKPREEQYNSTYRVVSVLTVLHQDWLNGKEY |
| | KCKVSNKALPAPIEKTISKAKGQPREPQVYTLPPS |
| | REEMTKNQVSLSCAVKGFYPSDIAVEWESNGQPEN |
| | NYKTTPPVLDSDGSFFLVSKLTVDKSRWQQGNVFS |
| | CSVMHEALHNHYTQKSLSLSPGK |

TABLE 7
FIRST POLYPEPTIDE: scFv-Fc fusion
CD3-tsm#7 (WT):

| | |
|---|---|
| Heavy chain | EVQLVQSGAEVKKPGASVKVSCKASGYTFISYTMH |
| variable | WVRQAPGQGLEWMGYINPRSGYTHYNQKLKDKATL |
| SEQ ID NO: 38 | TADKSASTAYMELSSLRSEDTAVYYCARSAYYDYD |
| | GFAYWGQGTLVTVSS |
| Linker | GGGGSGGGGSGGGGSGGGGS |
| SEQ ID NO: 23 | |
| Light chain | DIQMTQSPSSLSASVGDRVTITCSASSSVSYMNWY |
| variable | QQKPGKAPKRLIYDTSKLASGVPSRFSGSGSGTDF |
| SEQ ID NO: 39 | TLTISSLQPEDFATYYCQQWSSNPPTFGGGTKVEI |
| | K |
| Hinge: | GGSGGDKTHTCPPCPAPEAAGGPSV |
| SEQ ID NO: 25 | |
| CH2: | FLFPPKPKDTLMISRTPEVTCVVVDVSHEDPEVKF |
| SEQ ID NO: 26 | NWYVDGVEVHNAKTKPREEQYNSTYRVVSVLTVLH |
| | QDWLNGKEYKCKVSNKALPAPIEKTISKAK |
| CH3: | GQPREPQVYTLPPSREEMTKNQVSLSCAVKGFYPS |
| SEQ ID NO: 27 | DIAVEWESNGQPENNYKTTPPVLDSDGSFFLVSKL |
| | TVDKSRWQQGNVFSCSVMHEALHNHYTQKSLSLSP |
| | GK |
| Full-length | EVQLVQSGAEVKKPGASVKVSCKASGYTFISYTMH |
| sequence: | WVRQAPGQGLEWMGYINPRSGYTHYNQKLKDKATL |
| SEQ ID NO: 77 | TADKSASTAYMELSSLRSEDTAVYYCARSAYYDYD |
| | GFAYWGQGTLVTVSSGGGGSGGGGSGGGGSGGGGS |
| | DIQMTQSPSSLSASVGDRVTITCSASSSVSYMNWY |
| | QQKPGKAPKRLIYDTSKLASGVPSRFSGSGSGTDF |
| | TLTISSLQPEDFATYYCQQWSSNPPTFGGGTKVEI |
| | KGGSGGDKTHTCPPCPAPEAAGGPSVFLFPPKPKD |
| | TLMISRTPEVTCVVVDVSHEDPEVKFNWYVDGVEV |
| | HNAKTKPREEQYNSTYRVVSVLTVLHQDWLNGKEY |
| | KCKVSNKALPAPIEKTISKAKGQPREPQVYTLPPS |
| | REEMTKNQVSLSCAVKGFYPSDIAVEWESNGQPEN |
| | NYKTTPPVLDSDGSFFLVSKLTVDKSRWQQGNVFS |
| | CSVMHEALHNHYTQKSLSLSPGK |

TABLE 8
SECOND POLYPEPTIDE: heavy chain
CD38A2-3H10m1g1LALA-K:

| | |
|---|---|
| Heavy chain | QVQLVESGGGLVKPGGSLRLSCAASGFTFSDDYMS |
| variable | WIRQAPGKGLEWVASVSNGRPTTYYADSVRGRFTI |
| SEQ ID NO: 1 | SRDNAKNSLYLQMNSLRAEDTAVYYCAREGWSGEF |
| | TDWGQGTLVTVSS |
| Heavy chain | ASTKGPSVFPLAPSSKSTSGGTAALGCLVKDYFPE |
| constant | PVTVSWNSGALTSGVHTFPAVLQSSGLYSLSSVVT |
| SEQ ID NO: 2 | VPSSSLGTQTYICNVNHKPSNTKVDKRVEPKSC |
| Hinge: | DKTHTCPPCPAPEAAGGPSV |
| SEQ ID NO: 3 | |
| CH2: | FLFPPKPKDTLMISRTPEVTCVVVDVSHEDPEVKF |
| SEQ ID NO: 4 | NWYVDGVEVHNAKTKPREEQYNSTYRVVSVLTVLH |
| | QDWLNGKEYKCKVSNKALPAPIEKTISKAK |
| CH3: | GQPREPQVYTLPPSREEMTKNQVSLWCLVKGFYPS |
| SEQ ID NO: 5 | DIAVEWESNGQPENNYKTTPPVLDSDGSFFLYSKL |
| | TVDKSRWQQGNVFSCSVMHEALHNHYTQKSLSLSP |
| | GK |
| Full-length | QVQLVESGGGLVKPGGSLRLSCAASGFTFSDDYMS |
| sequence: | WIRQAPGKGLEWVASVSNGRPTTYYADSVRGRFTI |
| SEQ ID NO: 78 | SRDNAKNSLYLQMNSLRAEDTAVYYCAREGWSGEF |
| | TDWGQGTLVTVSSASTKGPSVFPLAPSSKSTSGGT |
| | AALGCLVKDYFPEPVTVSWNSGALTSGVHTFPAVL |
| | QSSGLYSLSSWTVPSSSLGTQTYICNVNHKPSNTK |
| | VDKRVEPKSCDKTHTCPPCPAPEAAGGPSVFLFPP |
| | KPKDTLMISRTPEVTCVVVDVSHEDPEVKFNWYVD |
| | GVEVHNAKTKPREEQYNSTYRVVSVLTVLHQDWLN |
| | GKEYKCKVSNKALPAPIEKTISKAKGQPREPQVYT |
| | LPPSREEMTKNQVSLWCLVKGFYPSDIAVEWESNG |
| | QPENNYKTTPPVLDSDGSFFLYSKLTVDKSRWQQG |
| | NVFSCSVMHEALHNHYTQKSLSLSPGK |

TABLE 9
SECOND POLYPEPTIDE: heavy chain
CD38A2g1LALA-K:

| | |
|---|---|
| Heavy chain variable | QVQLVESGGGLVKPGGSLRLSCAASGFTFSDDYMSWIRQAP |
| SEQ ID NO: 6 | GKGLEWVASVSNGRPTTYYADSVRGRFTISRDNAKNSLYL |
| | QMNSLRAEDTAVYYCAREDWGGEFTDWGQGTLVTVSS |
| Heavy chain constant | ASTKGPSVFPLAPSSKSTSGGTAALGCLVKDYFPEPVTVSW |
| SEQ ID NO: 7 | NSGALTSGVHTFPAVLQSSGLYSLSSVVTVPSSSLGTQTYIC |
| | NVNHKPSNTKVDKRVEPKSC |
| Hinge: | DKTHTCPPCPAPEAAGGPSV |
| SEQ ID NO: 3 | |
| CH2: | FLFPPKPKDTLMISRTPEVTCVVVDVSHEDPEVKFNWYVDG |
| SEQ ID NO: 4 | VEVHNAKTKPREEQYNSTYRVVSVLTVLHQDWLNGKEYK |
| | CKVSNKALPAPIEKTISKAK |
| CH3: | GQPREPQVYTLPPSREEMTKNQVSLWCLVKGFYPSDIAVE |
| SEQ ID NO: 5 | WESNGQPENNYKTTPPVLDSDGSFFLYSKLTVDKSRWQQG |
| | NVFSCSVMHEALHNHYTQKSLSLSPGK |
| Full-length sequence: | QVQLVESGGGLVKPGGSLRLSCAASGFTFSDDYMSWIRQAP |
| SEQ ID NO: 79 | GKGLEWVASVSNGRPTTYYADSVRGRFTISRDNAKNSLYL |
| | QMNSLRAEDTAVYYCAREDWGGEFTDWGQGTLVTVSSAS |
| | TKGPSVFPLAPSSKSTSGGTAALGCLVKDYFPEPVTVSWNS |
| | GALTSGVHTFPAVLQSSGLYSLSSWTVPSSSLGTQTYICNV |
| | NHKPSNTKVDKRVEPKSCDKTHTCPPCPAPEAAGGPSVFLF |
| | PPKPKDTLMISRTPEVTCVVVDVSHEDPEVKFNWYVDGVE |
| | VHNAKTKPREEQYNSTYRVVSVLTVLHQDWLNGKEYKCK |
| | VSNKALPAPIEKTISKAKGQPREPQVYTLPPSREEMTKNQVS |
| | LWCLVKGFYPSDIAVEWESNGQPENNYKTTPPVLDSDGSFF |
| | LYSKLTVDKSRWQQGNVFSCSVMHEALHNHYTQKSLSLSP |
| | GK |

TABLE 10
SECOND POLYPEPTIDE: heavy chain
CD38D8g1LALA-K:

| | |
|---|---|
| Heavy chain variable | QVQLVESGGGVVQPGGSLRLSCAASGFIVSTNYVHWVRQA |
| SEQ ID NO: 8 | PGKGLEWVSGIYSDPYTSYAYSDSVKGRFTISRDMSKNTVY |
| | LQMNRLRAEDTAVYYCARETNTGFSNSWYLDFWGQGTLV |
| | TVSS |
| Heavy chain constant | ASTKGPSVFPLAPSSKSTSGGTAALGCLVKDYFPEPVTVSW |
| SEQ ID NO: 9 | NSGALTSGVHTFPAVLQSSGLYSLSSVVTVPSSSLGTQTYIC |
| | NVNHKPSNTKVDKRVEPKSC |
| Hinge: | DKTHTCPPCPAPEAAGGPSV |
| SEQ ID NO: 3 | |
| CH2: | FLFPPKPKDTLMISRTPEVTCVVVDVSHEDPEVKFNWYVDG |
| SEQ ID NO: 4 | VEVHNAKTKPREEQYNSTYRVVSVLTVLHQDWLNGKEYK |
| | CKVSNKALPAPIEKTISKAK |
| CH3: | GQPREPQVYTLPPSREEMTKNQVSLWCLVKGFYPSDIAVE |
| SEQ ID NO: 5 | WESNGQPENNYKTTPPVLDSDGSFFLYSKLTVDKSRWQQG |
| | NVFSCSVMHEALHNHYTQKSLSLSPGK |
| Full-length sequence: | QVQLVESGGGVVQPGGSLRLSCAASGFIVSTNYVHWVRQA |
| SEQ ID NO: 80 | PGKGLEWVSGIYSDPYTSYAYSDSVKGRFTISRDMSKNTVY |
| | LQMNRLRAEDTAVYYCARETNTGFSNSWYLDFWGQGTLV |
| | TVSSASTKGPSVFPLAPSSKSTSGGTAALGCLVKDYFPEPVT |
| | VSWNSGALTSGVHTFPAVLQSSGLYSLSSWTVPSSSLGTQT |
| | YICNVNHKPSNTKVDKRVEPKSCDKTHTCPPCPAPEAAGGP |
| | SVFLFPPKPKDTLMISRTPEVTCVVVDVSHEDPEVKFNWYV |
| | DGVEVHNAKTKPREEQYNSTYRVVSVLTVLHQDWLNGKE |
| | YKCKVSNKALPAPIEKTISKAKGQPREPQVYTLPPSREEMTK |
| | NQVSLWCLVKGFYPSDIAVEWESNGQPENNYKTTPPVLDS |
| | DGSFFLYSKLTVDKSRWQQGNVFSCSVMHEALHNHYTQKS |
| | LSLSPGK |

TABLE 11
SECOND POLYPEPTIDE: heavy chain
CD38D8-3A1g1LALA-K:

| | |
|---|---|
| Heavy chain variable | QVQLVESGGGVVQPGGSLRLSCAASGFNVRTKYVHWVRQ |
| SEQ ID NO: 10 | APGKGLEWVSGIYSDPYTSYAYSDSVKGRFTISRDMSKNTV |
| | YLQMNRLRAEDTAVYYCARETNTGFSNSWYLDFWGQGTL |
| | VTVSS |
| Heavy chain constant | ASTKGPSVFPLAPSSKSTSGGTAALGCLVKDYFPEPVTVSW |
| SEQ ID NO: 11 | NSGALTSGVHTFPAVLQSSGLYSLSSVVTVPSSSLGTQTYIC |
| | NVNHKPSNTKVDKRVEPKSC |
| Hinge: | DKTHTCPPCPAPEAAGGPSV |
| SEQ ID NO: 3 | |
| CH2: | FLFPPKPKDTLMISRTPEVTCVVVDVSHEDPEVKFNWYVDG |
| SEQ ID NO: 4 | VEVHNAKTKPREEQYNSTYRVVSVLTVLHQDWLNGKEYK |
| | CKVSNKALPAPIEKTISKAK |
| CH3: | GQPREPQVYTLPPSREEMTKNQVSLWCLVKGFYPSDIAVE |
| SEQ ID NO: 5 | WESNGQPENNYKTTPPVLDSDGSFFLYSKLTVDKSRWQQG |
| | NVFSCSVMHEALHNHYTQKSLSLSPGK |
| Full-length sequence: | QVQLVESGGGVVQPGGSLRLSCAASGFNVRTKYVHWVRQ |
| SEQ ID NO: 81 | APGKGLEWVSGIYSDPYTSYAYSDSVKGRFTISRDMSKNTV |
| | YLQMNRLRAEDTAVYYCARETNTGFSNSWYLDFWGQGTL |
| | VTVSSASTKGPSVFPLAPSSKSTSGGTAALGCLVKDYFPEPV |
| | TVSWNSGALTSGVHTFPAVLQSSGLYSLSSVVTVPSSSLGTQ |
| | TYICNVNHKPSNTKVDKRVEPKSCDKTHTCPPCPAPEAAGG |
| | PSVFLFPPKPKDTLMISRTPEVTCVVVDVSHEDPEVKFNWY |
| | VDGVEVHNAKTKPREEQYNSTYRVVSVLTVLHQDWLNGK |
| | EYKCKVSNKALPAPIEKTISKAKGQPREPQVYTLPPSREEMT |
| | KNQVSLWCLVKGFYPSDIAVEWESNGQPENNYKTTPPVLD |
| | SDGSFFLYSKLTVDKSRWQQGNVFSCSVMHEALHNHYTQK |
| | SLSLSPGK |

TABLE 12
SECOND POLYPEPTIDE: heavy chain
CD38D8-3E1g1LALA-K:

| | |
|---|---|
| Heavy chain variable | QVQLVESGGGVVQPGGSLRLSCAASKLNVSPKYVHWVRQ |
| SEQ ID NO: 12 | APGKGLEWVSGIYSDPYTSYAYSDSVKGRFTISRDMSKNTV |
| | YLQMNRLRAEDTAVYYCARETNTGFSNSWYLDFWGQGTL |
| | VTVSS |
| Heavy chain constant | ASTKGPSVFPLAPSSKSTSGGTAALGCLVKDYFPEPVTVSW |
| SEQ ID NO: 13 | NSGALTSGVHTFPAVLQSSGLYSLSSVVTVPSSSLGTQTYIC |
| | NVNHKPSNTKVDKRVEPKSC |
| Hinge: | DKTHTCPPCPAPEAAGGPSV |
| SEQ ID NO: 3 | |
| CH2: | FLFPPKPKDTLMISRTPEVTCVVVDVSHEDPEVKFNWYVDG |
| SEQ ID NO: 4 | VEVHNAKTKPREEQYNSTYRVVSVLTVLHQDWLNGKEYK |
| | CKVSNKALPAPIEKTISKAK |
| CH3: | GQPREPQVYTLPPSREEMTKNQVSLWCLVKGFYPSDIAVE |
| SEQ ID NO: 5 | WESNGQPENNYKTTPPVLDSDGSFFLYSKLTVDKSRWQQG |
| | NVFSCSVMHEALHNHYTQKSLSLSPGK |
| Full-length sequence: | QVQLVESGGGVVQPGGSLRLSCAASKLNVSPKYVHWVRQ |
| SEQ ID NO: 82 | APGKGLEWVSGIYSDPYTSYAYSDSVKGRFTISRDMSKNTV |
| | YLQMNRLRAEDTAVYYCARETNTGFSNSWYLDFWGQGTL |
| | VTVSSASTKGPSVFPLAPSSKSTSGGTAALGCLVKDYFPEPV |
| | TVSWNSGALTSGVHTFPAVLQSSGLYSLSSVVTVPSSSLGTQ |
| | TYICNVNHKPSNTKVDKRVEPKSCDKTHTCPPCPAPEAAGG |
| | PSVFLFPPKPKDTLMISRTPEVTCVVVDVSHEDPEVKFNWY |
| | VDGVEVHNAKTKPREEQYNSTYRVVSVLTVLHQDWLNGK |
| | EYKCKVSNKALPAPIEKTISKAKGQPREPQVYTLPPSREEMT |
| | KNQVSLWCLVKGFYPSDIAVEWESNGQPENNYKTTPPVLD |
| | SDGSFFLYSKLTVDKSRWQQGNVFSCSVMHEALHNHYTQK |
| | SLSLSPGK |

TABLE 13
SECOND POLYPEPTIDE: heavy chain
CD38D8-1B5g1LALA-K:

| | |
|---|---|
| Heavy chain variable | QVQLVESGGGVVQPGGSLRLSCAASGLNARTNYVHWVRQ |
| SEQ ID NO: 14 | APGKGLEWVSGIYSDPYTSYAYSDSVKGRFTISRDMSKNTV |
| | YLQMNRLRAEDTAVYYCARETNTGFSNSWYLDFWGQGTL |
| | VTVSS |
| Heavy chain constant | ASTKGPSVFPLAPSSKSTSGGTAALGCLVKDYFPEPVTVSW |
| SEQ ID NO: 15 | NSGALTSGVHTFPAVLQSSGLYSLSSVVTVPSSSLGTQTYIC |
| | NVNHKPSNTKVDKRVEPKSC |
| Hinge: | DKTHTCPPCPAPEAAGGPSV |
| SEQ ID NO: 3 | |
| CH2: | FLFPPKPKDTLMISRTPEVTCVVVDVSHEDPEVKFNWYVDG |
| SEQ ID NO: 4 | VEVHNAKTKPREEQYNSTYRVVSVLTVLHQDWLNGKEYK |
| | CKVSNKALPAPIEKTISKAK |
| CH3: | GQPREPQVYTLPPSREEMTKNQVSLWCLVKGFYPSDIAVE |
| SEQ ID NO: 5 | WESNGQPENNYKTTPPVLDSDGSFFLYSKLTVDKSRWQQG |
| | NVFSCSVMHEALHNHYTQKSLSLSPGK |
| Full-length sequence: | QVQLVESGGGVVQPGGSLRLSCAASGLNARTNYVHWVRQ |
| SEQ ID NO: 83 | APGKGLEWVSGIYSDPYTSYAYSDSVKGRFTISRDMSKNTV |
| | YLQMNRLRAEDTAVYYCARETNTGFSNSWYLDFWGQGTL |
| | VTVSSASTKGPSVFPLAPSSKSTSGGTAALGCLVKDYFPEPV |
| | TVSWNSGALTSGVHTFPAVLQSSGLYSLSSVVTVPSSSLGTQ |
| | TYICNVNHKPSNTKVDKRVEPKSCDKTHTCPPCPAPEAAGG |
| | PSVFLFPPKPKDTLMISRTPEVTCVVVDVSHEDPEVKFNWY |
| | VDGVEVHNAKTKPREEQYNSTYRVVSVLTVLHQDWLNGK |
| | EYKCKVSNKALPAPIEKTISKAKGQPREPQVYTLPPSREEMT |
| | KNQVSLWCLVKGFYPSDIAVEWESNGQPENNYKTTPPVLD |
| | SDGSFFLYSKLTVDKSRWQQGNVFSCSVMHEALHNHYTQK |
| | SLSLSPGK |

TABLE 14
THIRD POLYPEPTIDE: light chain
CD38A2-3H10m1 LC:

| | |
|---|---|
| Light chain variable | QSALTQPPSASGTSGQRVTISCSGSSSNIGFHFVYWYQHLPG |
| SEQ ID NO: 16 | TAPKLLIYKNNQRPSGVPDRFSGSKSGNSASLAISGLRSEDE |
| | ADYYCAAWDDSLSGYVFGSGTKVTVL |
| Light chain constant | GQPKAAPSVTLFPPSSEELQANKATLVCLISDFYPGAVTVA |
| SEQ ID NO: 17 | WKADSSPVKAGVETTTPSKQSNNKYAASSYLSLTPEQWKS |
| | HRSYSCQVTHEGSTVEKTVAPTECS |
| Full-length sequence | QSALTQPPSASGTSGQRVTISCSGSSSNIGFHFVYWYQHLPG |
| SEQ ID NO: 84 | TAPKLLIYKNNQRPSGVPDRFSGSKSGNSASLAISGLRSEDE |
| | ADYYCAAWDDSLSGYVFGSGTKVTVLGQPKAAPSVTLFPP |
| | SSEELQANKATLVCLISDFYPGAVTVAWKADSSPVKAGVET |
| | TTPSKQSNNKYAASSYLSLTPEQWKSHRSYSCQVTHEGSTV |
| | EKTVAPTECS |

TABLE 15
THIRD POLYPEPTIDE: light chain
CD38A2 LC:

| | |
|---|---|
| Light chain variable | QSALTQPPSASGTSGQRVTISCSGSSSNIGINFVYWYQHLPGT |
| SEQ ID NO: 18 | APKLLIYKNNQRPSGVPDRFSGSKSGNSASLAISGLRSEDEA |
| | DYYCAAWDDSLSGYVFGSGTKVTVL |
| Light chain constant | GQPKAAPSVTLFPPSSEELQANKATLVCLISDFYPGAVTVA |
| SEQ ID NO: 19 | WKADSSPVKAGVETTTPSKQSNNKYAASSYLSLTPEQWKS |
| | HRSYSCQVTHEGSTVEKTVAPTECS |
| Full-length sequence | QSALTQPPSASGTSGQRVTISCSGSSSNIGINFVYWYQHLPGT |
| SEQ ID NO: 85 | APKLLIYKNNQRPSGVPDRFSGSKSGNSASLAISGLRSEDEA |
| | DYYCAAWDDSLSGYVFGSGTKVTVLGQPKAAPSVTLFPPSS |
| | EELQANKATLVCLISDFYPGAVTVAWKADSSPVKAGVETTT |
| | PSKQSNNKYAASSYLSLTPEQWKSHRSYSCQVTHEGSTVEK |
| | TVAPTECS |

TABLE 16
THIRD POLYPEPTIDE: light chain
CD38D8 LC

| | |
|---|---|
| Light chain variable | QPVLTQPPSASGTPGQRVTISCSGSSSNIGRNIVNWYQQLPG |
| SEQ ID NO: 20 | TTPKLLIYSNNQRPSGVPDRFSGSKSGTSASLAISGLHSEDEA |
| | DYYCATWDDSLNGWVFGGGTKLTVL |
| Light chain constant | GQPKAAPSVTLFPPSSEELQANKATLVCLISDFYPGAVTVA |
| SEQ ID NO: 21 | WKADSSPVKAGVETTTPSKQSNNKYAASSYLSLTPEQWKS |
| | HRSYSCQVTHEGSTVEKTVAPTECS |
| Full-length sequence | QPVLTQPPSASGTPGQRVTISCSGSSSNIGRNIVNWYQQLPG |
| SEQ ID NO: 86 | TTPKLLIYSNNQRPSGVPDRFSGSKSGTSASLAISGLHSEDEA |
| | DYYCATWDDSLNGWVFGGGTKLTVLGQPKAAPSVTLFPPS |
| | SEELQANKATLVCLISDFYPGAVTVAWKADSSPVKAGVETT |
| | TPSKQSNNKYAASSYLSLTPEQWKSHRSYSCQVTHEGSTVE |
| | KTVAPTECS |

TABLE 17
BZ1 heterodimeric antibody

| | |
|---|---|
| 1^{st} polypeptide | MEWSWVFLFFLSVTTGVHSQVQLVQSGAEVKKPGASVKVSCKASGYTFI |
| SEQ ID NO: 87 | SYTMHWVRQAPGQGLEWIGYINPRSGYTHYNQKLKDRATLTADKSASTA |
| | YMELSSLRSEDTAVYYCARSAYYDYDGFAYWGQGTLVTVSSGGGGSGGG |
| | GSGGGGSGGGGSDIQMTQSPSSLSASVGDRVTITCSASSSVSYMNWYQQ |
| | KPGKAPKRLIYDTSKLASGVPSRFSGSGSGTDFTLTISSLQPEDFATYY |
| | CQQWSSNPPTFGGGTKVEIKGGGGSDKTHTCPPCPAPEAAGGPSVFLFP |
| | PKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQ |
| | PREPQVYTLPPSREEMTKNQVSLSCAVKGFYPSDIAVEWESNGQPENNY |
| | KTTPPVLDSDGSFFLVSKLTVDKSRWQQGNVFSCSVMHEALHNHYTQKS |
| | LSLSPGK |
| 2^{nd} polypeptide | MEWSWVFLFFLSVTTGVHSQVQLVESGGGLVKPGGSLRLSCAASGFTFS |
| SEQ ID NO: 89 | DDYMSWIRQAPGKGLEWVASVSNGRPTTYYADSVRGRFTISRDNAKNSL |
| | YLQMNSLRAEDTAVYYCAREGWSGEFTDWGQGTLVTVSSASTKGPSVFP |
| | LAPSSKSTSGGTAALGCLVKDYFPEPVTVSWNSGALTSGVHTFPAVLQS |
| | SGLYSLSSVVTVPSSSLGTQTYICNVNHKPSNTKVDKRVEPKSCDKTHT |
| | CPPCPAPEAAGGPSVFLFPPKPKDTLMISRTPEVTCVVVDVSHEDPEVK |
| | FNWYVDGVEVHNAKTKPREEQYNSTYRVVSVLTVLHQDWLNGKEYKCKV |
| | SNKALPAPIEKTISKAKGQPREPQVYTLPPSREEMTKNQVSLWCLVKGF |
| | YPSDIAVEWESNGQPENNYKTTPPVLDSDGSFFLYSKLTVDKSRWQQGN |
| | VFSCSVMHEALHNHYTQKSLSLSPGK |
| 3^{rd} polypeptide | MSVPTQVLGLLLLWLTDARCQSVLTQPPSASGTSGQRVTISCSGSSSNI |
| SEQ ID NO: 88 | GFHFVYWYQHLPGTAPKLLIYKNNQRPSGVPDRFSGSKSGNSASLAISG |
| | LRSEDEADYYCAAWDDSLSGYVFGSGTKVTVLGQPKAAPSVTLFPPSSE |
| | ELQANKATLVCLISDFYPGAVTVAWKADSSPVKAGVETTTPSKQSNNKY |
| | AASSYLSLTPEQWKSHRSYSCQVTHEGSTVEKTVAPTECS |

TABLE 18
BZ1S heterodimeric antibody

| | |
|---|---|
| 1^{st} polypeptide | MEWSWVFLFFLSVTTGVHSQVQLVQSGAEVKKPGASVKVSCKASGYTFI |
| SEQ ID NO: 90 | SYTMHWVRQAPGQGLEWIGYINPRSGYTHYNQKLKDRATLTADKSASTA |
| | YMELSSLRSEDTAVYYCARSAYYDYDGFAYWGQGTLVTVSSGGGGSGGG |
| | GSGGGGSGGGGSDIQMTQSPSSLSASVGDRVTITCSASSSVSYMNWYQQ |
| | KPGKAPKRLIYDTSKLASGVPSRFSGSGSGTDFTLTISSLQPEDFATYY |
| | CQQWSSNPPTFGGGTKVEIKGGGGSDKTHTCPPCPAPEAAGGPSVFLFP |
| | PKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQ |
| | PREPQVYTLPPSREEMTKNQVSLSCAVKGFYPSDIAVEWESNGQPENNY |
| | KTTPPVLDSDGSFFLVSKLTVDKSRWQQGNVFSCSVMHEALHNHYTQKS |
| | LSLSPGK |
| 2^{nd} polypeptide | MEWSWVFLFFLSVTTGVHSQVQLVESGGGLVKPGGSLRLSCAASGFTFS |
| SEQ ID NO: 92 | DDYMSWIRQAPGKGLEWVASVSSGRPTTYYADSVRGRFTISRDNAKNSL |
| | YLQMNSLRAEDTAVYYCAREGWSGEFTDWGQGTLVTVSSASTKGPSVFP |
| | LAPSSKSTSGGTAALGCLVKDYFPEPVTVSWNSGALTSGVHTFPAVLQS |
| | SGLYSLSSVVTVPSSSLGTQTYICNVNHKPSNTKVDKRVEPKSCDKTHT |
| | CPPCPAPEAAGGPSVFLFPPKPKDTLMISRTPEVTCVVVDVSHEDPEVK |
| | FNWYVDGVEVHNAKTKPREEQYNSTYRVVSVLTVLHQDWLNGKEYKCKV |
| | SNKALPAPIEKTISKAKGQPREPQVYTLPPSREEMTKNQVSLWCLVKGF |
| | YPSDIAVEWESNGQPENNYKTTPPVLDSDGSFFLYSKLTVDKSRWQQGN |
| | VFSCSVMHEALHNHYTQKSLSLSPGK |
| 3^{rd} polypeptide | MSVPTQVLGLLLLWLTDARCQSVLTQPPSASGTSGQRVTISCSGSSSNI |
| SEQ ID NO: 91 | GFHFVYWYQHLPGTAPKLLIYKNNQRPSGVPDRFSGSKSGNSASLAISG |
| | LRSEDEADYYCAAWDDSLSGYVFGSGTKVTVLGQPKAAPSVTLFPPSSE |
| | ELQANKATLVCLISDFYPGAVTVAWKADSSPVKAGVETTTPSKQSNNKY |
| | AASSYLSLTPEQWKSHRSYSCQVTHEGSTVEKTVAPTECS |

TABLE 19
Anti-CD38 A2-SV variants

| Heavy chain variable region: | Light chain variable region: |
|---|---|
| CD38-A2 wild type SEQ ID NO: 40 | CD38-A2 wild type SEQ ID NO: 41 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GINFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREDWGGEFTDWGRGTLVTV | YCAAWDDSLSGYVFGSGTKVTVL |
| SS | |
| CD38-A2 SV mutant SEQ ID NO: 42 | CD38-A2 SV mutant SEQ ID NO: 43 |
| QVQLVESGGGLVKPGGSLRLSCAASGFTF | QSVLTQPPSASGTSGQRVTISCSGSSSNI |
| SDDYMSWIRQAPGKGLEWVASVSNGRPTT | GINFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREDWGGEFTDWGRGTLVTV | YCAAWDDSLSGYVFGSGTKVTVL |
| SS | |

TABLE 20
Anti-CD38 3H10 variants

| Heavy chain variable region: | Light chain variable region: |
|---|---|
| A2 parental heavy chain | A2 parental light chain |
| SEQ ID NO: 44 | SEQ ID NO: 45 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GINFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREDWGGEFTDWGRGTLVTV | YCAAWDDSLSGYVFGSGTKVTVL |
| SS | |
| 3H10m1 SEQ ID NO: 46 | 3H10m1 SEQ ID NO: 47 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GFHFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREGWSGEFTDWGQGTLVTV | YCAAWDDSLSGYVFGSGTKVTVL |
| SS | |
| 3G8m1 SEQ ID NO: 48 | 3G8m1 SEQ ID NO: 49 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GFHFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREAWGGEFTNWGQGTLVTV | YCAAWDDSLSGYVFGSGTKVTVL |
| SS | |
| 3E3m1 SEQ ID NO: 50 | 3E3m1 SEQ ID NO: 51 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GFHFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREAWGGEFTDWGQGTLVTV | YCAAWDDSLSGYVFGSGTKVTVL |
| SS | |
| 3G3 SEQ ID NO: 52 | 3G3 SEQ ID NO: 53 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GINFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREAWSGEFTDWGQGTLVTV | YCAAWDDSLSGYVFGSGTKVTVL |
| SS | |
| 3E11 SEQ ID NO: 54 | 3E11 SEQ ID NO: 55 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GINFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREGWGGEFTDWGQGTLVTV | YCAAWDDSLSGYVFGSGTKVTVL |
| SS | |
| 3H10 SEQ ID NO: 56 | 3H10 SEQ ID NO: 57 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GINFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREGWSGEFTDWGQGTLVTV | YCAAWDDSLSGYVFGSGTKVTVL |
| SS | |
| 3H10N SEQ ID NO: 58 | 3H10N SEQ ID NO: 59 |
| QVQLVESGGGLVKPGGSLRLSCAASGFTF | QSVLTQPPSASGTSGQRVTISCSGSSSNIG |
| SDDYMSWIRQAPGKGLEWVASVSNGRPTT | FHFVYWYQHLPGTAPKLLIYKNNQRPSGVP |
| YYADSVRGRFTISRDNAKNSLYLQMNSLR | DRFSGSKSGNSASLAISGLRSEDEADYYCA |
| AEDTAVYYCAREDWGGEFTDWGQGTLVTV | AWDDSLSGYVFGSGTKVTVL |
| SS | |
| 3H10NS SEQ ID NO: 60 | 3H10NS SEQ ID NO: 61 |
| QVQLVESGGGLVKPGGSLRLSCAASGFTF | QSVLTQPPSASGTSGQRVTISCSGSSSNIG |
| SDDYMSWIRQAPGKGLEWVASVSSGRPTT | FHFVYWYQHLPGTAPKLLIYKNNQRPSGVP |
| YYADSVRGRFTISRDNAKNSLYLQMNSLR | DRFSGSKSGNSASLAISGLRSEDEADYYCA |
| AEDTAVYYCAREDWGGEFTDWGQGTLVTV | AWDDSLSGYVFGSGTKVTVL |
| SS | |
| 3E10 SEQ ID NO: 62 | 3E10 SEQ ID NO: 63 |
| QVQLVESGGGLVKPGGSLRLSCAASGFTF | QAGLTQPPSASGTSGQRVTISCSGSSSNI |
| SDDYMSWIRQAPGKGLEWVASVSNGRPTT | GFHFVYWYQHLPGTAPKLLIYKNNQRPSG |
| YYADSVRGRFTISRDNAKNSLYLQMNSLR | VPDRFSGSKSGNSASLAISGLRSEDEADY |
| AEDTAVYYCAREDWGGEFTDWGRGTLVTV | YCAAWDDSLSGYVFGSGTKVTVL |
| SS | |
| 3H10m1g SEQ ID NO: 64 | 3H10m1g SEQ ID NO: 65 |
| QVQLVESGGGLVKPGGSLRLSCAASGFTF | QSVLTQPPSASGTSGQRVTISCSGSSSNIG |
| SDDYMSWIRQAPGKGLEWVASVSNGRPTT | FHFVYWYQHLPGTAPKLLIYKNNQRPSGVP |
| YYADSVRGRFTISRDNAKNSLYLQMNSLR | DRFSGSKSGNSASLAISGLRSEDEADYYCA |
| AEDTAVYYCAREGWSGEFTDWGQGTLVTV | AWDDSLSGYVFGSGTKVTVL |
| ss | |

TABLE 21
FIRST POLYPEPTIDE: scFv g4-Fc-fusion
CD3 with dent:

| | |
|---|---|
| Heavy chain variable | EVQLVQSGAEVKKPGASVKVSCKASGYTFISYTMHWVRQAPGQGL |
| SEQ ID NO: 94 | EWMGYINPRSGYTHYNQKLKDKATLTADKSASTAYMELSSLRSED |
| | TAVYYCARSAYYDYDGFAYWGQGTLVTVSS |
| Linker | GGGGSGGGGSGGGGS |
| SEQ ID NO: 95 | |
| Light chain variable | DIQMTQSPSSLSASVGDRVTITCSASSSVSYMNWYQQKPGKAPKR |
| SEQ ID NO: 96 | LIYDTSKLASGVPSRFSGSGSGTDFTLTISSLQPEDFATYYCQQW |
| | SSNPPTFGGGTKVEIK |
| Hinge: | GGGGSSKYGPPCPPCPAPEFLGGPSV |
| SEQ ID NO: 97 | |
| CH2: | FLFPPKPKDTLMISRTPEVTCVVVDVSQEDPEVQFNWYVDGVEVH |
| SEQ ID NO: 98 | NAKTKPREEQFNSTYRVVSVLTVLHQDWLNGKEYKCKVSNKGLPS |
| | SIEKTISKAK |
| CH3: | GQPREPQVYTLPPSQEEMTKNQVSLTCLVKGFYPSDIAVEWESNG |
| SEQ ID NO: 99 | QPENNYKTTPPVLDSDGSFLLYSRLTVDKSRWQEGNVFSCSVMHE |
| with dent | ALHNHYTQKSLSLSLGK |
| Full-length sequence: | EVQLVQSGAEVKKPGASVKVSCKASGYTFISYTMHWVRQAPGQGL |
| SEQ ID NO: 100 | EWMGYINPRSGYTHYNQKLKDKATLTADKSASTAYMELSSLRSED |
| | TAVYYCARSAYYDYDGFAYWGQGTLVTVSSGGGGSGGGGSGGGGS |
| | DIQMTQSPSSLSASVGDRVTITCSASSSVSYMNWYQQKPGKAPKR |
| | LIYDTSKLASGVPSRFSGSGSGTDFTLTISSLQPEDFATYYCQQW |
| | SSNPPTFGGGTKVEIKGGGGSSKYGPPCPPCPAPEFLGGPSVFLF |
| | PPKPKDTLMISRTPEVTCVVVDVSQEDPEVQFNWYVDGVEVHNAK |
| | TKPREEQFNSTYRVVSVLTVLHQDWLNGKEYKCKVSNKGLPSSIE |
| | KTISKAKGQPREPQVYTLPPSQEEMTKNQVSLTCLVKGFYPSDIA |
| | VEWESNGQPENNYKTTPPVLDSDGSFLLYSRLTVDKSRWQEGNVF |
| | SCSVMHEALHNHYTQKSLSLSLGK |

TABLE 22
SECOND POLYPEPTIDE: IgG4 heavy chain
CD38D8-1B5g1LALA-K (with bump):

| | |
|---|---|
| Heavy chain variable | QVQLVESGGGVVQPGGSLRLSCAASGFIVSTNYVHWVRQAPGKGL |
| SEQ ID NO: 101 | EWVSGIYSDPYTSYAYSDSVKGRFTISRDMSKNTVYLQMNRLRAE |
| | DTAVYYCARETNTGFSNSWYLDFWGQGTLVTVSS |
| Heavy chain constant | ASTKGPSVFPLAPCSRSTSESTAALGCLVKDYFPEPVTVSWNSGA |
| SEQ ID NO: 102 | LTSGVHTFPAVLQSSGLYSLSSVVTVPSSSLGTKTYTCNVDHKPS |
| | NTKVDKRVESKYG |
| Hinge: | PPCPPCPAPEFLGGPSV |
| SEQ ID NO: 103 | |
| CH2: | FLFPPKPKDTLMISRTPEVTCVVVDVSQEDPEVQFNWYVDGVEVH |
| SEQ ID NO: 104 | NAKTKPREEQFNSTYRVVSVLTVLHQDWLNGKEYKCKVSNKGLPS |
| | SIEKTISKAK |
| CH3: | GQPREPQVYTLPPSQEEMTKNQVSLTCLVKGFYPSDIAVEWESNG |
| SEQ ID NO: 105 | QPENNYKTTPPVLDSDGSFFLYSRLTVDKSRWQEGNVFSCSVMHE |
| with bump | ALHNHYTQKSLSLSLGK |
| Full-length sequence: | QVQLVESGGGVVQPGGSLRLSCAASGFIVSTNYVHWVRQAPGKGL |
| SEQ ID NO: 106 | EWVSGIYSDPYTSYAYSDSVKGRFTISRDMSKNTVYLQMNRLRAE |
| | DTAVYYCARETNTGFSNSWYLDFWGQGTLVTVSSASTKGPSVFPL |
| | APCSRSTSESTAALGCLVKDYFPEPVTVSWNSGALTSGVHTFPAV |
| | LQSSGLYSLSSVVTVPSSSLGTKTYTCNVDHKPSNTKVDKRVESK |
| | YGPPCPPCPAPEFLGGPSVFLFPPKPKDTLMISRTPEVTCVVVDV |
| | SQEDPEVQFNWYVDGVEVHNAKTKPREEQFNSTYRVVSVLTVLHQ |
| | DWLNGKEYKCKVSNKGLPSSIEKTISKAKGQPREPQVYTLPPSQE |
| | EMTKNQVSLTCLVKGFYPSDIAVEWESNGQPENNYKTTPPVLDSD |
| | GSFFLYSRLTVDKSRWQEGNVFSCSVMHEALHNHYTQKSLSLSLG |
| | K |

Anti-CD38 IgG1: heavy chain constant region:
SEQ ID NO: 66
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Anti-CD38 IgG1-SPPC: heavy chain constant region:
SEQ ID NO: 67
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTSPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Anti-CD38 IgG4: heavy chain constant region:
SEQ ID NO: 68
ASTKGPSVFPLAPCSRSTSESTAALGCLVKDYFPEPVTVSWNSGALTSGV
HTFPAVLQSSGLYSLSSWTVPSSSLGTKTYTCNVDHKPSNTKVDKRVESK
YGPPCPPCPAPEFLGGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGK
Anti-CD38: kappa light chain constant region:
SEQ ID NO: 69
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
Antigen: CD38 protein:
SEQ ID NO: 70
VPRWRQQWSGPGTTKRFPETVLARCVKYTEIHPEMRHVDCQSVWDAFKGA
FISKHPCNITEEDYQPLMKLGTQTVPCNKILLWSRIKDLAHQFTQVQRDM
FTLEDTLLGYLADDLTWCGEFNTSKINYQSCPDWRKDCSNNPVSVFWKTV
SRRFAEAACDWHVMLNGSRSKIFDKNSTEGSVEVHNLQPEKVQTLEAWVI
HGGREDSRDLCQDPTIKELESIISKRNIQFSCKNIYRPDKFLQCVKNPED
SSCTSEI
Antigen: human CD3 epsilon (UniProt P07766-1)
(SEQ ID NO: 93)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI

### EXAMPLES

The following examples are meant to be illustrative and can be used to further understand embodiments of the present disclosure and should not be construed as limiting the scope of the present teachings in any way.

### Example 1: Bispecific Antibody Expression

A transient CHO cell expression system by Thermo Fisher (FreeStyle CHO-S) was used to express each CD38/CD3 IgG-scFv bispecific antibody. CHO-S cells were thawed and revived from liquid nitrogen in Free Style 293 Expression Medium and slowly adapted to CHO-SFM II Medium. CHO-S cells can be further grown in CHO-SFM II Medium at no higher than 4×10\hat{ }6/mL cell density. A cell density between 1-2×10\hat{ }6/mL and viability greater than 97% such CHO-S cell culture was used for transient transfection with the IgG-scFv BsAb constructs.

Following manufacturer's protocol, each CD38/CD3 IgG-scFv chain DNA construct in pSTI expression vector was prepared using the NucleoBond Xtra Midi EF DNA Preparation Kit (Macherey-Nagel). Equal DNA input ratio between the three DNA constructs coding an anti-CD38 light chain, an anti-CD38 heavy chain with Knob and LALA mutations, and an anti-CD3-scFv Fc fusion chain with Hole and LALA mutations, respectively, were packed with PEI at 1:3 mass ratio prior to CHO transfection. A 2 ug of total DNA input per mL of CHO-S cells were used for transfection. PEI-packed CD38/CD3 IgG-scFv BsAb constructs was added directly into the CHO-S culture described above. The transfected CHO-S cell culture was placed in on an orbital shaker in a 37° C. cell culture incubator with 5% CO2 and let recovery overnight. An equal volume of CD FortiCHO medium as the transfected CHO-S culture was added the next day to quench the transfection and the transfected culture moved to an orbital shaker in a 28° C. cell culture incubator with 5% CO2 for several days.

Continued bispecific antibody titer and cell viability was monitor throughout the continued CHO expression period. The culture supernatant was harvested when the cell viability dropped below 70% or no continued titer increase observed in 2-3 consecutive days. The bispecific antibody titer was measured using a Protein A biosensor tip on the Octet system using manufacturer recommended protocol and standards. Cell viability was measured using a 1:1 (v:v) dilution of the cell culture with 0.2% Trypan Blue solution on the Contess II FL cell counter.

### Example 2: Bispecific Antibody Downstream Purification Method Development

The GMP compatible downstream purification methods for further polishing Protein A affinity liquid chromatography-purified CD38/CD3 IgG-scFv bispecific antibody were developed using the AKTApure and the AKTA Avant FPLC instruments. A screening of multiple CIEX and HIC GMP grade liquid chromatography media were performed using the built-in scouting suite of AKTA software Unicorn. Up to 5 CIEX or HIC media were screened at a time with up to 2 buffer sets, for a total of 10 combinations of conditions, for each tested CD38/CD3 IgG-scFv candidate/lead were screened. The FPLC runs were set at 1 mL/min flow rate for sample loading, column washing, and elution. A typical 100% B in 20 c.v. gradient elution method was used for both CIEX and HIC media screening. Several sets of buffer conditions were also further tested once a promising media was selected based on media screening results. CIEX run buffers were Na Acetate pairs with the same pH, and several NaCl concentrations at loading and washing were screened for the purpose to improve yield, segregation of impurity, and process efficiency. HIC run buffers were Na Acetate or BTP pairs with the same pH, and several (NH₄)₂SO₄ concentrations at loading and washing were screened for the purpose to improve yield, segregation of impurity, and process efficiency. The elution peak fractions in bind-then-elute mode method, or the flow-through fractions in the passing-by mode method were subjected to SDS-PAGE analysis using the Bio-Rad Mini-PROTEAN® Tetra Vertical Electrophoresis Cell electrophoresis apparatus and the Bio-Rad 4-20% Mini-PROTEAN® TGX™ Precast Protein Gels. Samples were mixed with NuPAGE™ LDS Sample Buffer (4×) and heated at 85° C. for 2 min prior to gel loading.

An SEC method was also developed using the Superdex 200 Increase 10/300 GL preparation grade SEC column at 0.5 mL/min elution in the SEC buffer to polish protein A-purified CD38/CD3 IgG-scFv bispecific antibodies.

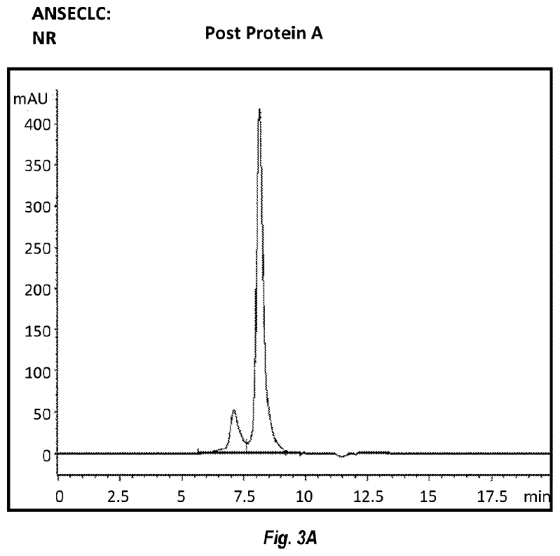

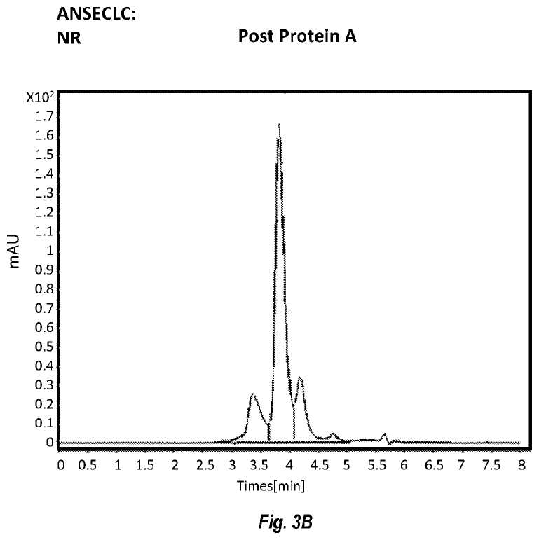

An SDS-PAGE gel of Protein A purified CD38/CD3 bispecific antibody throughout several iterations of protein engineering is shown in FIG. 2. A size exclusion chromatogram of bispecific antibody CD38A2-3H10/CD3tsm #1 knob-into-hole is shown in FIG. 3A. A size exclusion chromatogram of bispecific antibody CD38D8/ss4×CD3 bump-into-dent is shown in FIG. 3B.

### Example 3: Thermal-Stable Anti-CD3 (Hum291) scFv Fragment (for First Polypeptide Chains)

The heavy chain variable region of hum291 anti-CD3 antibody was engineered for increased thermal stability using error-prone PCR to introduce single, double or triple mutations (see SEQ ID NOS:22, 28, 30, 32, 34, and 36; SEQ ID NO: 38 is the wild type Hum291 heavy chain variable region sequence).

Mutagenesis and Phage Library Construction:

The Diversity PCR Random Mutagenesis kit (Takara, catalog No. 630703) was used to incorporate mutations in HuM291 CD3-scFv coding frame. Condition 5 in the User Manual provided by the manufacturer was used to generate approximately 5 nucleotides per 1000 bp. The Diversify PCR products were cloned into phage a library, which was called ‘TSM’ phage library.

Thermo-Challenging and Panning:

A standard panning protocol was used to pan the CD3-scFv TSM Phage Library with the following modifications. Two rounds of thermo-challenge and panning alternated with primary T cells and Jurkat cells as CD3 antigen presenter were performed using 3 different conditions: (1) 1st—50° C.×1 hour, panned on T cells, 2^{nd}—50° C.×1 hour, panned on Jurkat cells; (2) 1st—55° C.×1 hour, panned on T cells, 2^{nd}—58° C.×1 hour, panned on Jurkat cells; and (3) 1st—65° C.×1 hour, panned on T cells, 2^{nd}—65° C.×1 hour, panned on Jurkat cells. Up to 96 clones per condition were picked and the CD3-scFv mutants periplasmic were mini-prepped.

Phage Library Screening:

The mini-prepped mutant CD3-sc-Fv from the selected phage clones was either directly incubated with Jurkat cells in a 96-well plate well or heated at 58° C. for 30 minutes prior to the incubation with Jurkat cells in a separate 96-well plate. Flow cytometry analysis of these CD3-scFv mutants binding to CD3 on Jurkat cells was performed on Intellicyte IQue Screener Plus flow cytometer using an anti-human Fc secondary antibody to detect clones with unaltered binding histogram between the heated and the unheated conditions. A wild-type HuM291 CD3-scFv in heated and unheated conditions was also included in the Jurkat binding screening as a control to establish the distinct difference between denatured and functional native CD3-scFv binders before and after the thermo-challenge.

Analysis of Hits from the TSM Phage Library:

CD3-scFv TSM hit phage clones with similar Jurkat cell binding behaviors before and after heat treatment were identified and sequenced. Mutational hot spots outside of the CDRs were analyzed for their potential positive structural stability impact. CD3-scFv TSM hit sequences were total synthesized to exclude the CDR mutations, if any, for cloning into expression vector and subsequent small-scale transient CHO cell expression. The expressed CD3-scFv TSM hits were individually purified by protein A affinity liquid chromatography, and their Tm analyzed by intrinsic Trp fluorescence shift assay using the UNcle instrument. Intrinsic tryptophan fluorescence shift measurements were used to determine thermal stability of wild-type and six mutant anti-CD3 scFv fragments. The resulting Tm values are listed in Table 25 below:

TABLE 25

| | | Anti-CD3 EC5 |
|---|---|---|
| Anti-CD3 scFv fragment: | Tm: | ug/mL |
| Anti-CD3 1^{st} gen. SM | 63.5° C. | 0.2 |
| CD3-tsm#l ( (M48I/K67R) | 65° C. | 0.2 |
| CD3-tsm#2 (I30V/K67R) | 64.6° C. | 1.5 |
| CD3-tsm#3 (M48I) | 62.8° C. | 0.2 |
| CD3-tsm#4 (I30V/M48I/K67R) | 65.8° C. | 0.1 |
| CD3-tsm#5 (K67R) | 64.5° C. | 0.1 |
| CD3-tsm#6 ( (BOV) | 60.6° C. | 0.1 |
| CD3-tsm#7 (WT) | 60° C. | 0.2 |
| BZI1 pool 1 PA/SEC | 64.3° C. | |
| BZ1/GV3/BF1 PA/SEC | 65.7° C. | |
| BZ1S pool 2 PA/SEC | 63.8° C. | |

After testing the bispecific antibodies for thermal stability, some of the CD38/CD3 series of IgG-scFv bispecific antibodies were designed to consist of IgG4 heavy chain with CH3 domain interface engineering or IgG1 heavy chain with CH3 domain interface engineering in combination of one of two hinge mutation strategies. The IgG4 CH3 domain interface engineering utilized two strategies, denoted as Bump-into-Dent (g4CH3a-WT/g4CH3b-F405L), and Knob-into-Hole (g4CH3a-T366W/g4CH3b-T366S-L368A-Y407V), respectively, to drive heavy chain heterodimerization. The IgG1 CH3 domain interface engineering utilizes the Knob-into-Hole (g1CH3a-T366W/g1CH3b-SA-T366V) strategy only. The IgG1 hinge mutation strategy one is L234A-L235A, and strategy 2 is L234F-L235E-D265A, the latter of which is used as a reference.

In another CD38/CD3 series of IgG-scFv bispecific antibodies, a collection of CD38 paratopes were engineered into the Fab domain of the IgG-scFv construct with the CD38 heavy chain comprising the Bump and/or the Knob mutation in gamma-1 or gamma-4 constructs. The CD3 paratope was fashioned into an scFv domain with a 20 amino acid-long linker composed of a blend of Glycine and Serine residues connected the VH and VL domains of the scFv. The CD3-scFv is fused into the N-terminus of the hinge of the Dent and/or Hole mutation-containing Fc chain (scFv-Fc fusion) of the gamma-4 or gamma-1 chain. A 5-amino acid-long linker composed of a blend of Glycine and Serine was also used to space the C-terminus of the CD3-scFv domain and the hinge for added flexibility. The CD3-scFv may further contain stability engineering mutations such as Vh44C/V1100C, or VhK67R/M48I.

Characterizing Lead Hits:

A statistical analysis was applied with the Design of Experiment principles on the impact of mutation(s) over total Tm gain in TSM Hits was performed using the software MiniTab. A mutational descriptor matrix was generated from tabulating the Tm and positional mutations from the CD3-scFv TSM hits. The correlation of each descriptor vs Tm gain was calculated and evaluated by line plot and its slope to predict the likelihood of achieving substantial thermostability gain when a mutational descriptor is present. The influential mutation(s) and their positional effect, as well as combinatory effect were determined based on their correlation to a greater Tm gain as collected in the UNcle run of the TSM library hits.

A subset of 6 “Focus Group” hits, with different combination of the high influential mutations, were selected to validate their Tm gain and to examine the validity of the statistical analysis. The sequences of the Focus Group hits were synthesized and the CD3-scFv g1 Fc fusion chains constructed. The CD3-scFv TSM Focus Group hits were subsequently transiently expressed in CHO-S cells and purified for Tm and CD3 affinity confirmation by UNcle and flowcytometry, respectively.

The CD3-scFv TSM Leads were selected from this Focus Group based on the following characteristics: (1) having the highest Tm gain, 5° C., in this subset of hits; (2) achieving the highest Tm gain with the least number of mutations; (3) having most mutations buried than exposed comparing to other Focus Group hits; and (4) having the least side chain chemistry change in the exposed mutation residue.

Results: After four iterations of engineering of the anti-CD3 (hum291) scFv fragment (for the first polypeptide chains), the chain association of the first, second and third chain improved. The heterodimeric species was the dominant species with reduced HMW and LMW species after a one-step protein A affinity purification (see FIG. 2).

### Example 4: Analytical Analysis Via SEC

Size exclusion chromatography (SEC) was performed on the CD38/CD3 IgG-scFv bispecific engineering samples, using TSKgel SuperSW mAb HR column (4 μm, 7.8 mm×300 mm) and a mobile phase of 0.2 M Potassium Phosphate, 0.25 M KCl, pH 6.2, at a flow rate of 0.8 mL/min. Fifty (50) μL of diluted sample at 1 mg/mL was loaded onto the column. Data were monitored and collected at 280 nm by an ultraviolet (UV) detector. See FIGS. 3A and B.

### Example 5: Analytical Analysis Via CE-SDS

Reducing and non-reducing capillary electrophoresis sodium dodecyl sulfate (CE SDS) were performed on the CD38/CD3 IgG-scFv bispecific antibodies using Agilent Bioanalyzer 2100 following the SOP 8018. Agilent Protein 230 kit was used for protein analysis.

### Example 6: Analytical Analysis of Tm Via DSC

The CD38/CD3 IgG-scFv bispecific antibodies were protein A purified and kept at 4° C. in 125 mM Na Acetate pH 5 prior to buffer exchange into SEC buffer using a centrifugal concentrator of 10 KD cutoff to reach 1 mg/mL final concentration. MicroCal VP-DSC instrument was set with the following parameters: starting temperature at 30° C., final temperature at 90° C., scan rate at 60° C./hr, pre-scan thermostat 10 minutes, and post-scan thermostat 0 minutes. Twelve cycles of water run were performed prior to buffer and bispecific antibody sample measurement. After subtracting the buffer baseline from the sample heatflux vs temperature curve, the Tm was identified as the phase transition peak of the curve, with the first peak Tm1, second peak Tm2, an so on.

### Example 7: Analytical Analysis of Tm by Intrinsic Trp Fluorescence Shift

Uncle analysis was conducting according to manufacturer's instruction, 9 uL/sample/run with triplicate were loaded into each sample slot of the UNcle chip (from Unchained Labs, catalog #EQN1642). The Tm/Tagg measurement suite in the UNcle instrument was set up with the following parameters: starting temperature at 25° C.; final temperature at 90° C.; scan rate 4 fluorescence data acquisition per ° C. temperature change; temperature ramping rate 1° C./min; pre-scan thermostat 10 minutes; and post-scan thermostat 0 minutes.

The BCM (barycentric mean) of the Trp fluorescence peak at each temperature was determined by the UNcle analysis suite and plotted again the temperature ramp. Tm is identified from the differential of the BCM vs temperature curve as the temperature where a peak, phase transition, occurs. The lowest temperature where such a peak occurs is defined as Tm1, the next temperature up where such a peak occurs as Tm2, and so on.

### Example 8: Primary T Cell Binding Assays

Flow cytometry-based primary T cell binding assays were used to measure the binding affinity of CD38/CD3 IgG-scFv bispecific antibodies towards cell surface CD3 antigen. The binding of CD3 antigen on primary human T cells by the CD38/CD3 IgG-scFv bispecific antibodies were performed on Intellicyt IQu Screener Plus flow cytometer. Freshly isolated human T cells were negatively selected again for the CD38(−) subpopulation prior to the experiment. T cells with greater than 90% viability were washed with FACS buffer, a DPBS based buffer containing 2% FBS once and adjusted to 1E+6/mL cell density in the same buffer. Approximately 25,000 cells were used to generate each data point. For each dose-dependent curve of bispecific antibody or control mAb binding to CD3 on CD38(−) T cells, a series of 1:3 dilution, up to twelve times, of each bispecific antibody or the control mAb were prepared in the FACS buffer. Equal volume of each bispecific antibody dilution and the T cells were mixed in a well of a 96-well plate. After one hour of incubation of each concentration series of bispecific antibody or mAb with their corresponding T cells, the mixtures were washed with FACS buffer and supernatant removed after centrifugation. A mouse anti-human Fc-APC conjugate as secondary antibody were added into each bispecific antibody or mAb/cell mixture at the manufacturer recommended titer and incubated for approximately 15-20 minutes. The bispecific (or mAb)/cell mixtures were then washed and supernatant removed as described above. The final bispecific/cell mixtures were resuspended in 25 uL of FACS buffer and subjected to FACS analysis on the flow cytometer with 10 uL of each suspension analyzed.

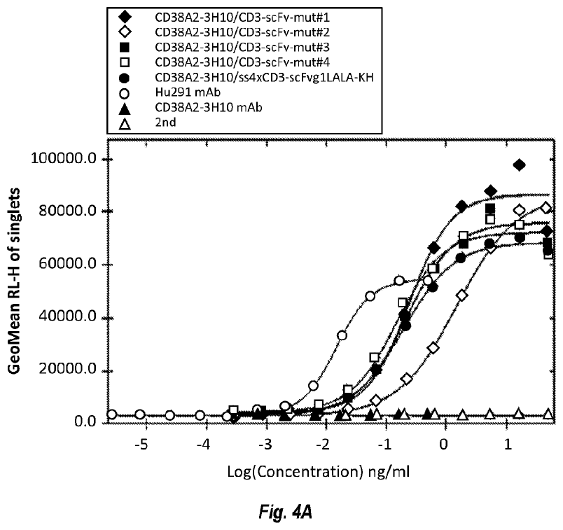

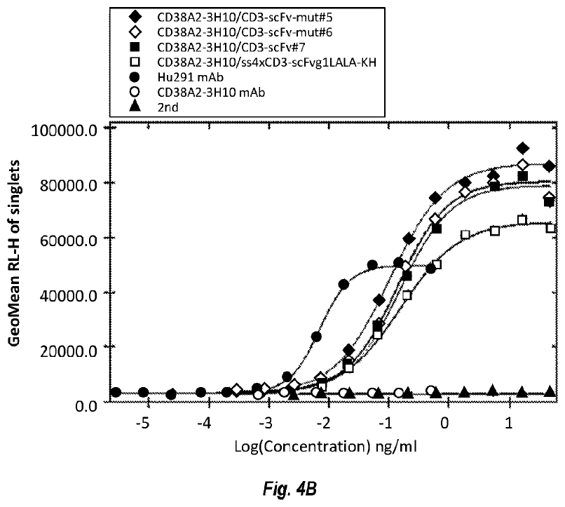

The dose-dependent binding curve of each bispecific antibody or control mAb binding to T cells were generated by plotting the Geomean of the fluorescence height of the detected singlet cells in the corresponding bispecific (or mAb)/cell mixture in each concentration series. EC50 of each bispecific or control mAb towards CD3 on T cells was determined after fitting the curve to the dose-dependent curve with four-parameter linear regression (4PL) model. The results shown in FIGS. 4A and B indicate that after protein engineering of the anti-CD3 scFv, the CD3 affinity was retained.

### Example 9: Affinity Determination Via SPR

Biacore T200 instrument was utilized to measure the affinity of CD38/CD3 bispecific antibodies and control anti-CD38 monoclonal antibody towards recombinant CD38 antigen. Kinetic interactions between bispecific antibodies and CD38 antigen were measured at 25° C. using Biacore T200 surface plasmon resonance (GE Healthcare). Anti-human fragment crystallizable (Fc) antibody (Human Antibody Capture Kit) was immobilized on a Series S CM5 sensor chip to approximately 10000 RU using standard N-Hydroxysuccinimide/1-ethyl-3-(-3-dimethylaminopropyl) carbodiimide hydrochloride (NHS/EDC) coupling methodology. Antibodies (bispecific or mAb, approximately 2 μg/mL) were captured for 60 seconds at a flow rate 10 μL/min. Recombinant human CD38-his tag was serially diluted in running buffer (1×HBS-EP+). All measurements were conducted with a flow rate of 30 μL/min. Surfaces were regenerated with 3M MgCl₂ for 60 s. A 1:1 (Langmuir) binding model was used to fit the data.

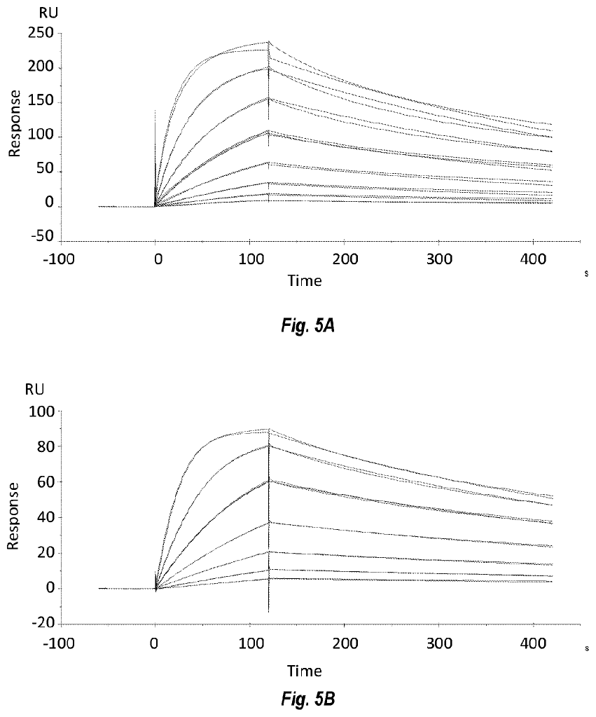

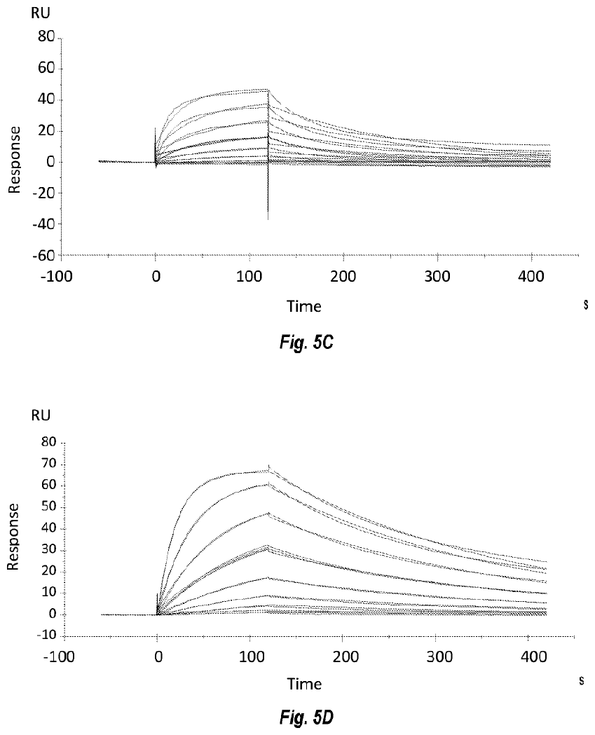

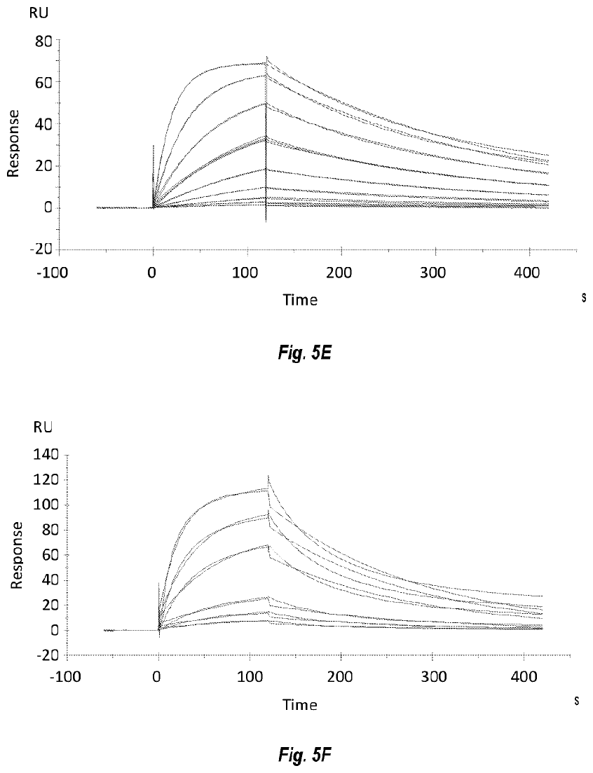

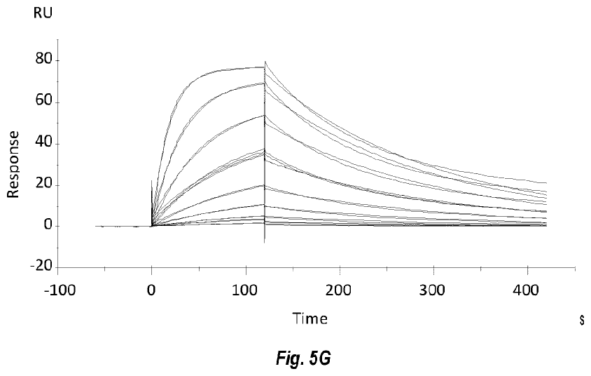

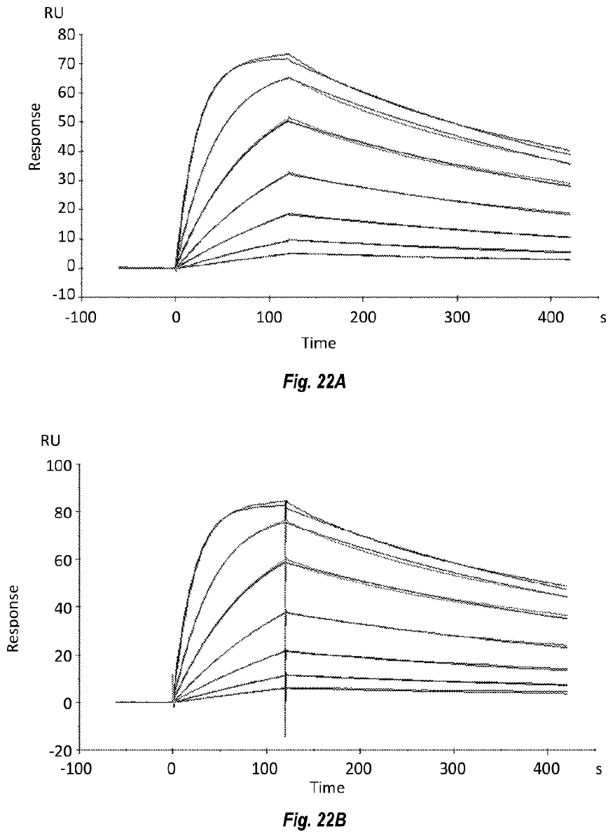

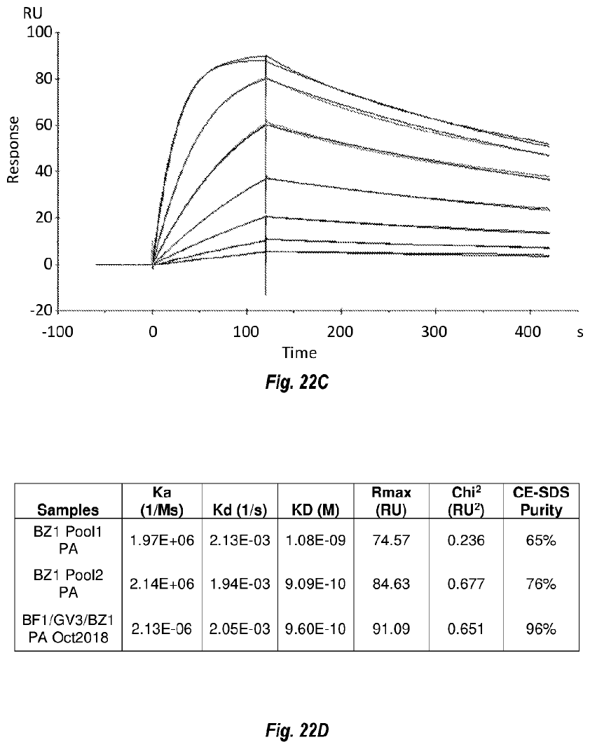

Biacore binding kinetics of the CD38×CD3 bispecific antibodies towards each individual antigen as the first and sole binding event were obtained for the target combination: CD38/CD3. The bispecific antibody was immobilized onto a biosensor surface via amine coupled anti human Fc antibodies on a CM5 sensor chip (GE Healthcare) according to the manufacturer's recommendation. A concentration series, ranging from 0 to approximately 10× of the KD, of each individual antigen was applied as the analytes to the biosensor surface that was immobilized with the corresponding bispecific antibody or mAb for 2 minutes for the association phase, followed by a buffer flow of 5 minutes for the dissociation phase in cycles. The sensorgrams of individual bispecific molecule binding to its corresponding antigens are presented in FIGS. 5A-G and FIGS. 22A-C. Some kinetics analysis of the parental mAb (anti-CD38) was performed with 200 seconds association phase and 300 seconds dissociation phase.

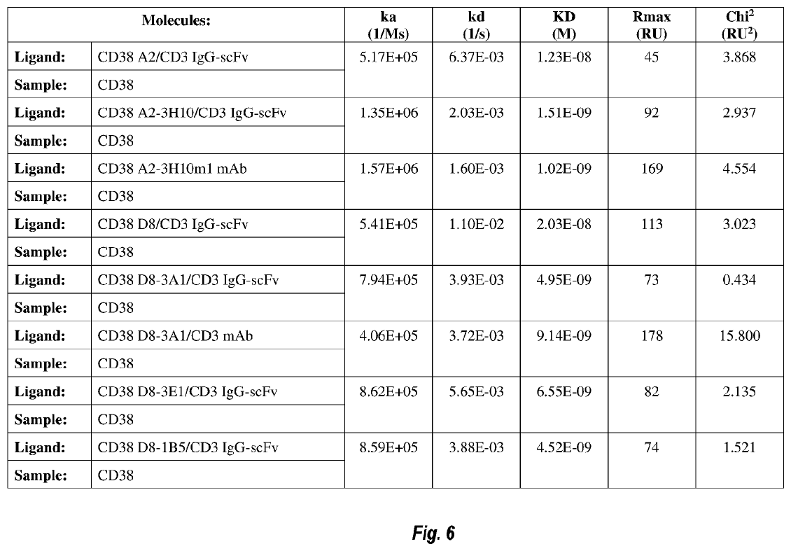

A standard evaluation with the 1:1 binding model was applied to all kinetics series described above. The association and dissociation constants (k-on and k-off), the affinity (KD), maximal response (Rmax), as well as the fitting statistics (Chi\hat{ }2) are presented in the Table shown in FIG. 6.

FIGS. 5A-G show sensorgrams of binding kinetics between CD38 antigen and various bispecific antibodies. The binding kinetic values are listed in the Table 26 below, where ‘H’ designates high affinity, ‘M’ designates moderate affinity, and ‘L’ designates low affinity.

TABLE 26

| | | CD38 | CD3 |
|---|---|---|---|
| Figure: | CD38/CD3 antibody: | Affinity: | Affinity: |
| 22A | BZ1 | H | M |
| 22D | CD38 D8-1B5/CD3 (M48I/K67R) | M | M |
| 22B | CD38 A2/CD3 (M48I/K67R) | M | M |
| 22E | CD38 D8/CD3 (M48I/K67R) | L | M |
| 22C | CD38 D8-3A1/CD3 (M48I/K67R) | M | M |
| 22F | CD38 D8-3E1/CD3 (M481/K67R) | M | M |
| | H: | ≤2 nM | ≤2 nM |
| | M: | 2-20 nM | 2-20 nM |
| | L: | 20-200 nM | 20-200 nM |

Determining Fc Effector Functions of Bispecific Antibodies with LALA Mutation:

BIAcore T200 instrument was also used to measure the affinity of CD38/CD3 bispecific antibodies and control mAbs, with wild-type hinge or hinge mutations, as well as glycol-engineered, towards recombinant CD16A high and low affinity variants. Commercially available (AcroBiosystem) biotinylated CD16A 176V high affinity, or 176F low affinity, variant via the C-terminal Avi-tag was individually immobilized onto a flow cell surface of a SA Sensor Chip a Series S to approximately 160 RU using standard streptavidin-biotin immobilization methodology. Antibodies (bispecific or mAb, approximately 2 μg/mL) were each serially diluted in running buffer (1×HBS-EP+). All measurements were conducted with a flow rate of 30 μL/min. Surfaces were regenerated with 3M MgCl₂ for 60 seconds. A Steady-state binding affinity model was used to fit the data. The steady-state response determined by this fitting process was exported and used to perform a Michaelis-Menten function fit of the steady-state response vs [Ab] in logarithmic scale using GraphPad.

Using the SA-immobilized biotinylated CD16A high affinity variant V158 and low affinity variant F158, the affinity of a CD38/CD3 bispecific antibody (having a first, second and third chain combination: SEQ ID NOS:71, 89 and 84, respectively) with the LALA hinge mutation, as well as several other control mAbs, with wild-type hinge, CH2 domain mutation, or glycoengineered, were determined by both the Biacore kinetics analysis and the steady-state response analysis as well. The latter when fitted with the Michalis-Menten function, produces the Km that is mathematically equivalent to the KD of a protein-ligand binding event. The Rmax from the Michaelis-Menten fit represents the maximal response in the CD161A engagement and the level of change of total achievable binding efficiency of the bispecific antibody or mAb.

The SPR results demonstrated that the LALA mutation in the bispecific antibody hinge region drastically reduced affinity of the CD38/CD3 bispecific antibody for both high affinity (176V) and low affinity (176F) variants of CD16A, compared to IgG1 mAbs having wild type hinge region (anti-CD47-F7, anti-CTLA4-F7), and compared to mAbs having a variant SPPC hinge (anti-BCMA-2C5) and an mAb having a LALA mutation (anti-BCMA-2C5) (data not shown).

### Example 10: Re-Directed T Cell Cytotoxicity (RTCC)

Determining CD38 Expression Level in Tumor Cell Lines:

Six CD38(+) tumor cell lines were assessed for their relative CD38 expression level: RPMI 8226, MM.1R, IM-9, Raji, Daudi, and NCI-H929. Each of the six cell lines were cultured to sub-confluent in cell culture flasks the day prior to CD38 antigen staining. Cells with greater to 90% viability were titrated to 1E+6 per mL density and seeded in a V-bottom 96-well plate at 50,000 cells per well. The cells were washed and resuspended in FACS buffer with anti-CD38 APC-conjugate (Biolegend) at manufacturer recommended concentration along with designated unstained controls. The cells were incubated at room temperature in the dark for 20 minutes. The cells were washed and resuspended in FACS buffer prior to flow cytometry analysis.

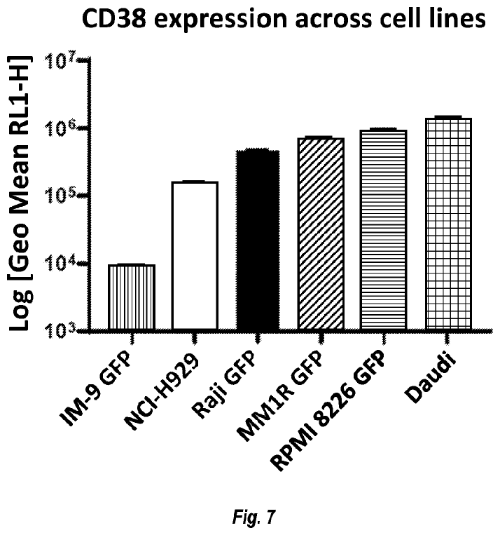

Flow cytometer (Intellicyte IQue Screener, Sartorius) was set up with RL-1 channel for anti-CD38-APC detection. Cells were identified using the FSC-H (forward scattering fluorescence height) vs SSC-H (side scattering fluorescence-height) scatter plot of all events. The cell population was then plotted with FCS-H vs FCS-A (area) to identify the singlets. The geocentric mean of the RL1-H channel histogram of the singlets for each tumor cell line was exported and plotted as representation of CD38 expression level using GraphPad Prism software. The results are shown in FIG. 7.

Bispecific Antibody-Mediated Tumor Cell Killing by Un-Stimulated Human T Cells:

The ability of the CD38/CD3 bispecific antibodies to redirect human T cells to kill tumor cells with CD38 antigens was assessed by subjecting equal amount of human T cells and tumor cells at a fixed E/T (effector cells/target cells) ratio to a concentration gradient of each CD38/CD3 bispecific antibody overnight and quantifying the apoptosis marker presence on the tumor cells thereafter. Freshly isolated human T cells were kept in RPMI media, supplemented with 10% FBS and 10 ng/mL IL-7 at approximately 2-5E+6 cells/mL at 37° C. static incubator overnight. Prior to the cytotoxicity assay setup, an assay of cytotoxic versus helper T cell ratio was conducted by quantifying the percentage of CD4(+) and CD8(+) subpopulations, respectively, in total CD3(+) T cell counts using a compensated three-color flow cytometry method (see description below).

RPMI8226, a CD38 high-expressing human multiple myeloma cell line, was labeled by transduction with pMYs-IRES, a retrovirus-derived vector with GFP and firefly luciferase genes and subsequent selection for a stable cell line of endogenous GFP and luciferase expression for fluorescence as well as luminescence detection. RPMI GFP-Luc cell and freshly isolated T cells of viability >90% on the day of the assay setup was each washed with RPMI media once and density adjusted to achieve an E/T ratio of 20:1 in each assay condition, with approximately 15,000 target cells used per data point. A serial 1:3 dilution of each CD38/CD3 bispecific antibody, as well as the mixture of the two parental mAb, was prepared in RPMI media and added to the RPMI/T cell mixtures in 96-well plates. A series of target cell and T cell only wells were also set up as the secondary antigen only and no-bispecific antibody controls. RPMI only and T cell only wells were also included to assess the intrinsic apoptosis baseline of each cell type.

The assay plates were incubated at 37° C. overnight. Plates were centrifuged to pellet the cells and 100 uL of the assay supernatants were transferred from each well into a new 96-well PCR tube plates to free at −80° C. for cytokine content analyses at separate time. The cell pellets were washed with FACS buffer once prior to Annexin V APC-conjugate staining and analysis by flowcytometry. The APC Annexin V staining was performed according to manufacturer's recommendation and procedure.

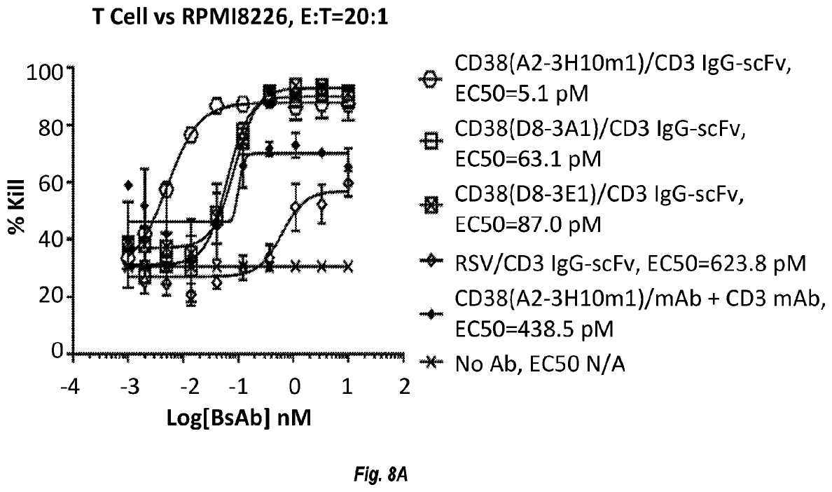

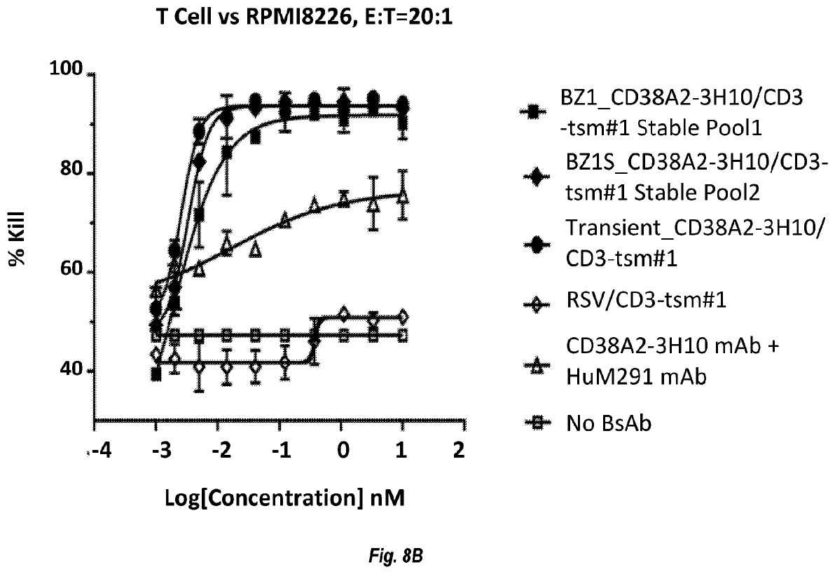

The assay plates were then subjected to FACS analysis on the Intellicyte IQue Plus Screener flow cytometer with BL-1 channel for GFP and RL-1 channel Annexin V detection. The RPMI GFP/Fluc target cells are distinguishable from T cells on the FSC-A (forward scattering-area) vs BL1-H (height) scatter plot, with the BL1-H high population being the target cells, the BL1-H low the effector cells. The RPMI cells were further gated on the RL1-H (Annexin V) vs BL1-H scatter plot, with the RL1-H high population being the Annexin V high expression subset. The % Kill of the target cells is defined as %[Total target cell count minus the Annexin V low subset count]/[Total target cell count] in each assay condition. The % Kill of RMPI is then plotted against the bispecific antibody or control mAb concentration gradient. The EC50 is defined as the concentration of the biological agent needed to produce 50% of the maximal observable effect for the defined experimental condition. The results are shown in FIGS. 8A and B.

Quantifying Sub-Populations of CD4+ and CD8+ T Cells in Un-Stimulated Human PBMCs:

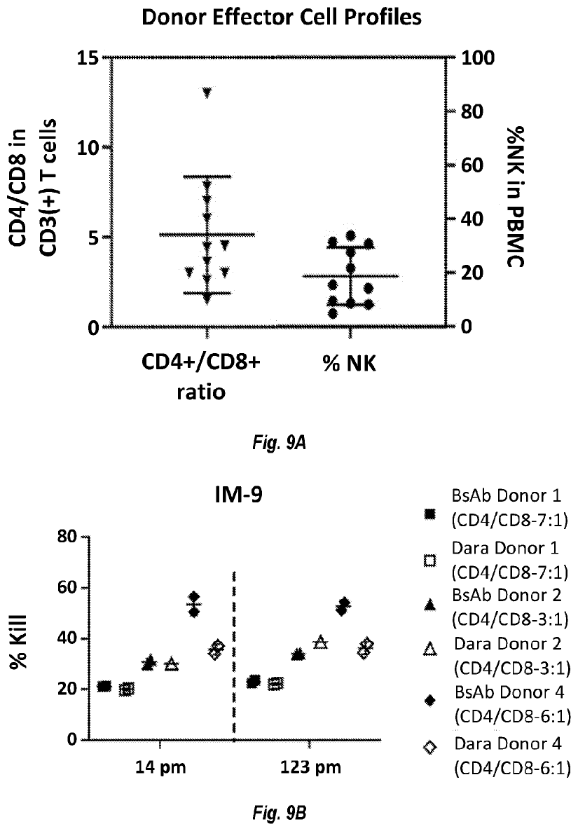

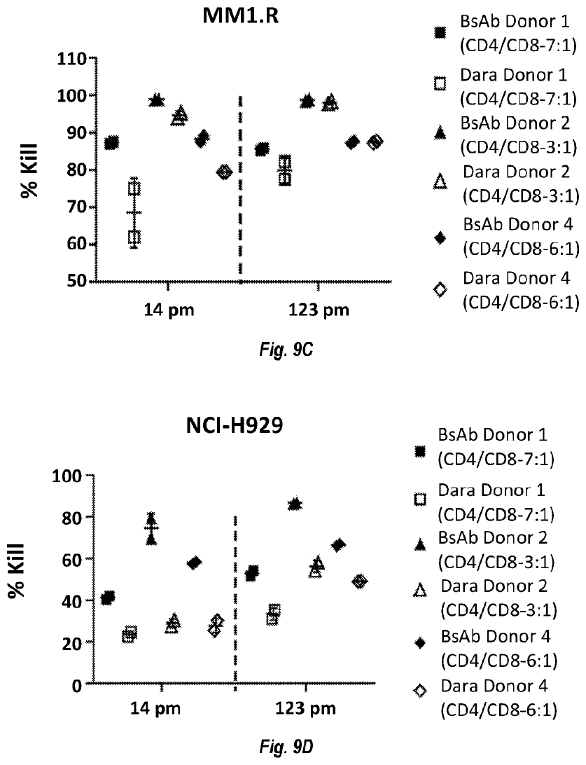

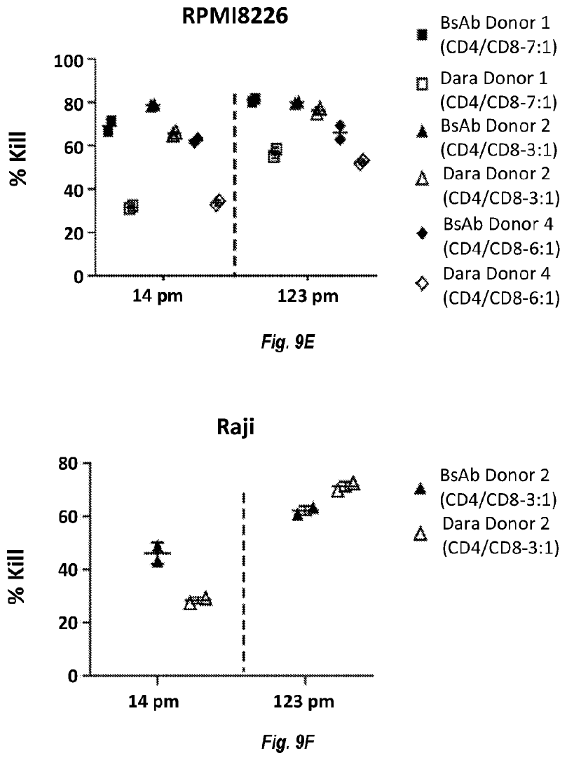

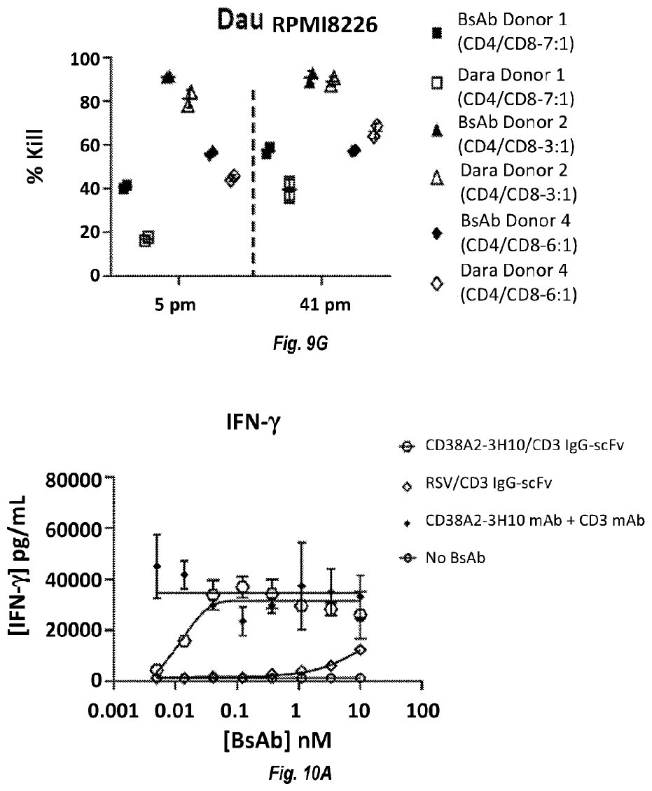

Prior to the RTCC assay described above, freshly isolated human PBMCs were kept in RPMI media, supplemented with 10% FBS and 10 ng/mL IL-7 at approximately 2-5E+6 cells/mL at 37° C. static incubator overnight. Prior to the cytotoxicity assay setup (described below), an assay of cytotoxic versus helper T cell ratio were conducted by quantifying the percentage of CD4(+) and CD8(+) subpopulations, respectively, in total CD3(+) T cell counts using a compensated three-color flowcytometry method. A parallel assay of NK cell content in isolated PBMCs by a compensated two-color flow cytometry method staining CD3(+) for T cell and CD56 (+) for NK was also performed. The results are shown in FIG. 9A.

Bispecific Antibody-Mediated Tumor Cell Killing by Un-Stimulated Human PBMC Cells:

The efficiencies of CD38/CD3 bispecific antibodies to redirect primary human T cells to kill CD38-expressing tumor cell lines were assessed by subjecting equal amount of freshly isolated human PBMCs and each CD38(+) tumor cell lines, at a fixed E/T ratio, to a concentration gradient of CD38/CD3 bispecific antibody in 96-well plates overnight at 37° C. Six CD38(+) tumor cell lines (RPMI 8226, MM.1R, IM-9, Raji, Daudi, and NCI-H929) were subjected to this in vitro efficacy assessment. To compare the potency of redirected T cell cytotoxicity (RTCC) mediated by CD38/CD3 bispecific antibody with that of antibody-dependent cell-mediated cytotoxicity (ADCC), an NK cell mediated CD38-targeting commercial mAb drug Darzalex-dependent tumor cell killing phenomenon, the same concentration gradient of Darzalex was also used treat similar PBMCs/CD38(+) tumor cells mixture in parallel. Apoptosis marker presence on the tumor cells were quantified the next day using a flow cytometry method.

The tumor cell lines (RPMI 8226, MM.1R, IM-9, and Raji) were labeled by transduction with pMYs-IRES, a retrovirus-derived vector with GFP and firefly luciferase genes and subsequent selection for a stable cell line of endogenous GFP and luciferase expression for fluorescence detection. These cells were cultured to maintain a good viability prior to assay setup. NCI-H929 and Daudi cells was labeled using CFSE the day before assay day and washed prior to assay setup.

The freshly isolated PBMCs, as well as the GFP-expressing and CD38 antigen level-characterized tumor cells of viability >90% on the day of the assay setup were washed with RPMI media once and density adjusted to achieve an E/T ratio of 10:1 for target cells RPMI8226, IM-9, NCI-H929, Daudi, and MA/11.R, and 40:1 for Raji cells, with approximately 15,000 target cells per data point. A serial 1:3 or 1:4 dilution of the anti-CD38/CD3 bispecific antibody or Darzalex (anti-CD38 mAb), was prepared in RPMI media and added to the E/T cell mixtures in each 96-well plates. A series of target cell and T cell only wells were also setup as the secondary antigen only and no-bispecific antibody controls. Tumor cells only and PBMCs only wells were also included to assess the intrinsic apoptosis baseline of each cell type. To address the edge effect of plate-based assays, only the inner rows and columns of the 96-well plates were used for setting the assays. The border rows and columns were filled with equal volumes of the RPMI media as the inner assay wells. The assay plates were than kept in at a 37° C. static incubator overnight.

Cells from the overnight incubation were pelleted by centrifugation. The cell pellets were washed with FACS buffer once and the centrifuged prior to supernatant removal. The cell pellets in each assay plate were then subjected to Annexin V detection and analysis. The Annexin V detection was performed using a commercially available FACS kit by BioLegend, Inc, following manufacturer recommended conditions and procedures.

The assay plates were then subjected to FACS analysis on the Intellicyte IQue Screener Plus flow cytometer with BL-1 channel for GFP and RL-1 channel Annexin V detection. Due to the constitutive expression of GFP, the tumor target cells can be distinguished from the effector cells on the FSC-A (forward scattering-area) vs BL1-H scatter plot, with the BL1-H high population being the target cells, the BL1-H low the effector cells. The GFP(+) tumor cells were further gated on the RL1-H (Annexin V) vs BL1-H scatter plot, with the RL1-H high population being the Annexin V high subset, and the RL1-H low population the Annexin V low subset. The % Kill of the target cells is defined as the percentage of the (Total Target Cell counts of the GFP high in the 0 concentration-treated condition−Annexin V low subset) over the Total Target Cell counts of the GFP high in the 0 concentration-treated condition in each assay condition. The % Kill of each assay well was plotted against the CD38/CD3 bispecific antibody or Darzalex concentration gradient.

The 5-parameter non-linear regression (5-PL) fitted dose-dependent response model with asymmetrical top/bottom fitting strategy (S factor) was used to fit these % Kill vs Log [Concentration] plots in GraphPad Prism. PMBCs from multiple donors were profiled against each of the six CD38(+) tumor cells in duplicates. The results are shown in FIGS. 9B-G.

### Example 11: Cytokine Release Assay

A multiplex MSD (Meso Scale Diagnostics) method was used to analyze cytokine release capability of CD38/CD3 heterodimeric (bispecific) antibodies. Unstimulated human T cells and RPMI8226 cells were mixed with various concentrations of CD38/CD3 bispecific antibodies or control antibodies in the redirected T cell cytotoxicity assays. Three cytokines were tested, including IFNγ, IL-2, and TNFα, in a T cell cytotoxicity which were assayed simultaneously for each redirected T cell cytotoxicity condition. The manufacturer recommended assay procedure was used in carrying out the assays with the following sample handling practice.

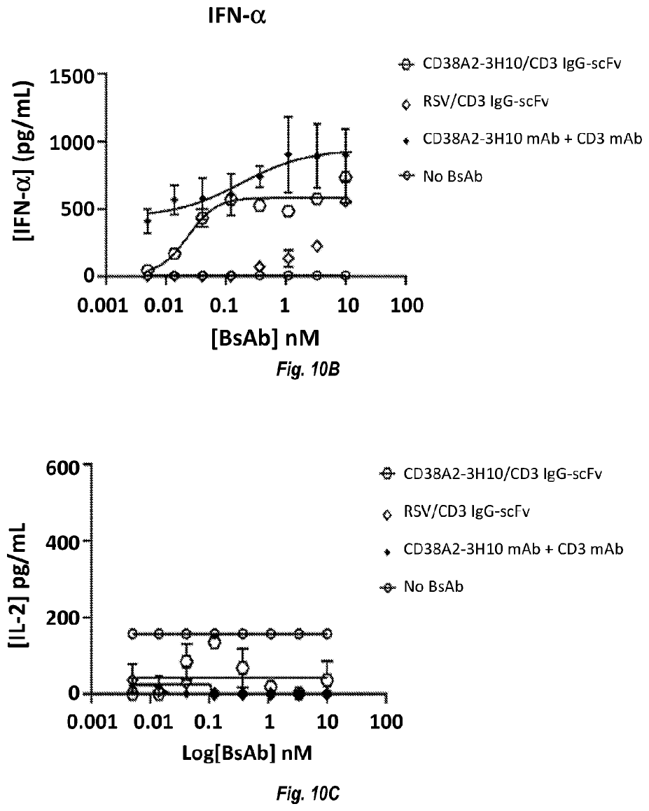

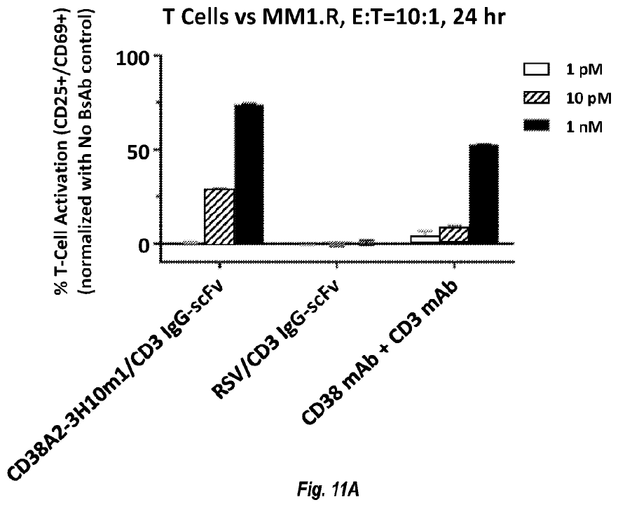

The previously frozen supernatants were thawed at room temperature and homogenized by inversion and brief centrifugation. For the series treated with bispecific antibody or control mAb concentration gradients, where tumor target cells RPMI killings were observed, the homogenized supernatants were diluted 50 times prior to MSD plate loading. For the series as cell-only controls, i.e. tumor target cells only, T cells only, or the two-cell mixtures, where less cell death was observed and less cytokines were expected, the supernatants were diluted 10 times prior to MSD plate loading. The results are shown in FIGS. 10A-C.

### Example 12: In Vitro T Cell Activation Assay

The ability of CD38/CD3 bispecific antibodies to activate previously unstimulated human T cells upon the stimulation of tumor cells that express CD38 or BCMA antigens was assessed by subjecting the mixture of human T cells and tumor cells at a fixed E/T (effector cells/target cells) ratio to each bispecific antibody at concentrations above or below the previous determined EC50 of the corresponding bispecific antibody in redirected T cell cytotoxicity (described above). In this T cell activation assay, the CD38(+) myeloma cell line MM1.R GFR/luc was used to present the TAA to the freshly isolated human T cells in the presence of each CD38/CD3 bispecific antibody, as well as parent mAb controls. Roughly 15,000 MM1.R cells and 150,000 primary human T cells per assay condition was incubated at 37° C. static incubator overnight in a 96-well plate in the presence of 1 pM, 10 pM, or 1 nM of each bispecific antibody or mAb control. T cell only was also included in the set-up in parallel with each of the corresponding bispecific antibody or control mAb condition as antigen-free control. Cell pellets from overnight incubation were pelleted by centrifugation and washed once with the RPMI media+10% FBS prior to FACS staining and analysis. Cell pellets were resuspended in FACS buffer and double stained with anti CD25-APC and anti CD69-APC-Cy7 antibodies (BioLegend, Inc.) according to manufacturer recommended assay titer and procedure. Proper compensation control for double-color FACS assay as well as no-Ab controls were also included in the assay design.

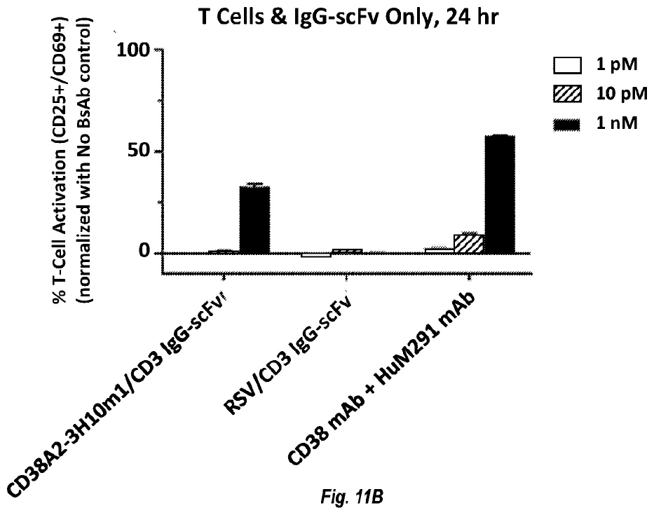

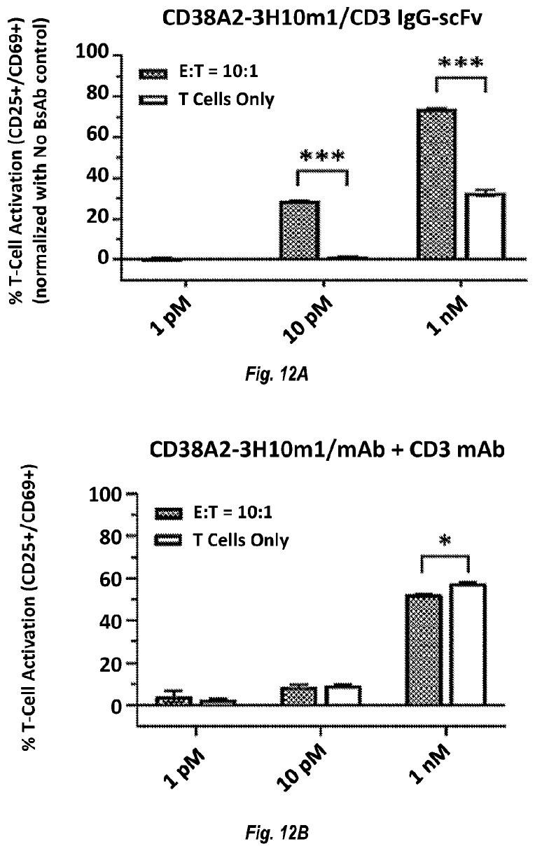

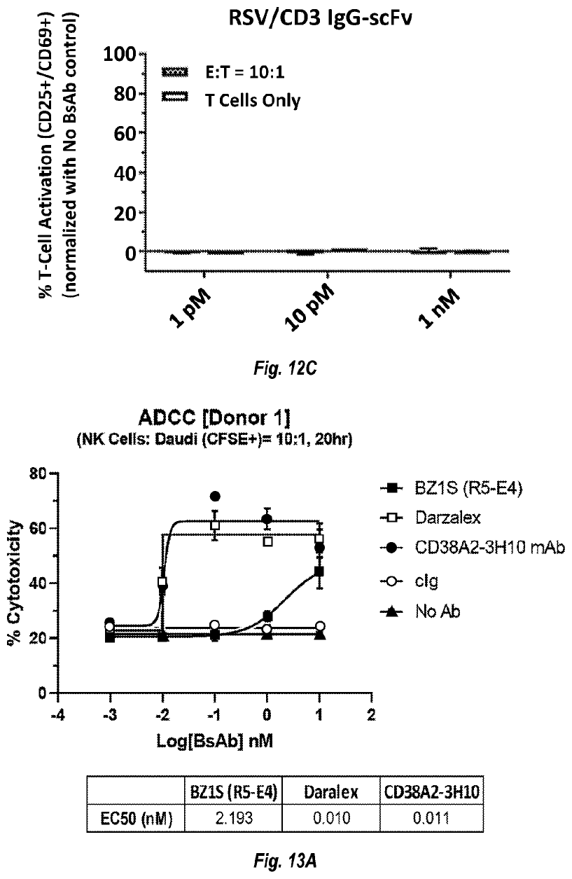

Prior to FACS analysis, the washed and double-stained cell pellets were resuspended in FACS buffer. Approximately ⅓ of (or 50,000-60,000) cells per assay condition were analyzed by the FACS instrument on BL-1 (GFP), RL-1 (CD25-APC), and RL-2 (CD69-APC-Cy7) channels. T cells were gated from the MA/11.R target cells on the FSC-A (forward scattering-area) vs BL1-H (height) scatter plot, with the BL1-H low population as the effector cells. The T cell population was then plotted on the RL-1 (CD25-APC) vs RL-2 (CD69-APC-Cy7) scatter plot after proper compensation was taken. The upper right quadrant where both CD25(+) and CD69(+) are highly expressed, was defined as the activated T cell population. The percentage of the CD25(+)/CD69(+) T cell in all detected T cell population was calculated as the % Activation for the corresponding assay condition. The % Activation per assay condition is then normalized by subtracting the level of activation observed in target cells and T cells only condition. The results are shown in FIGS. 11A and B, and FIGS. 12A-C. The bispecific antibody (CD38A2-3H10m1/CD3tsm #1 IgG-scFv) mediates TAA-dependent activation of previously unstimulated T cells in a dose-dependent manner (FIG. 11A). In the absence of tumor target, the bispecific antibody induces T cell activation only to a much less degree (FIG. 11B).

### Example 13: ADCC Assays

In this ADCC assay, Daudi cells were used as CD38-expressing target cells to perform the assay, which are labeled with CellTrace™ CFSE dye (Invitrogen) previous to assay set up.

The effector population in the ADCC assay consists of NK cells, which were prepared from peripheral blood mononuclear cells using a human NK cell enrichment kit (StemCell Technologies). Once prepared, the NK cells were either used straight or cultured in RPMI 1640+10% FBS containing IL-2 (50 U/mL) at 37° C. and 5% CO2 for assay set up the next day. The NK cells were washed before use by centrifugation at 500 g for 5 minutes then resuspended in RPMI 1640+10% FBS and counted using a hemocytometer.

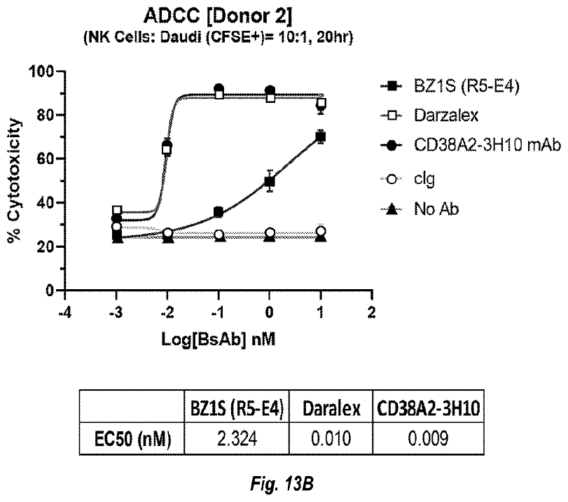

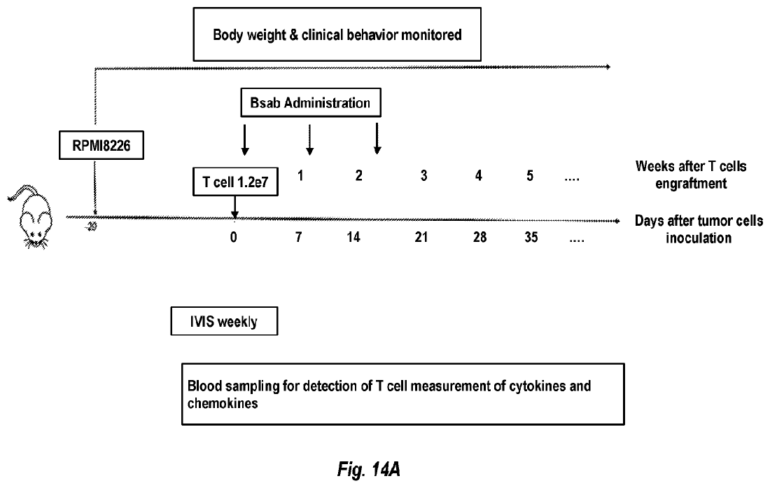

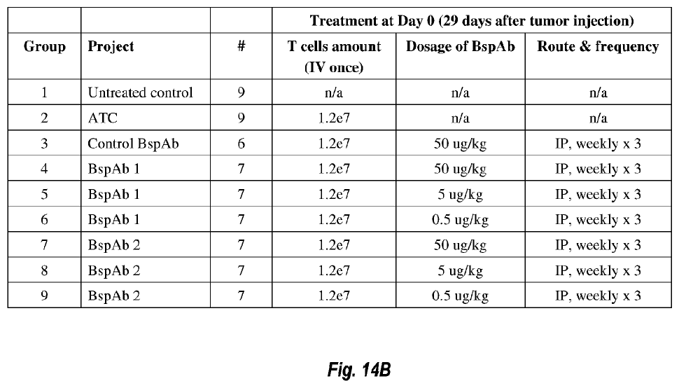

The bispecific antibody BZ1S (clone R5-E4), Darzalex, or CD38 3H10m1 mAb were added at 10 nM or indicated dilutions thereof to 1.5×10⁴ CFSE labeled Daudi cells in 120 μL RPMI 1640+10% FBS in the wells of a 96-well plate. After incubating 30 minutes at 37° C. and 5% CO2, 60 uL of 1.5×10⁵ NK cells were then added to give an effector to target ratio of 10:1. The cells were incubated at 37° C. and 5% CO2 for overnight, followed by staining using the LIVE/DEAD™ Fixable Yellow Dead Cell Stain Kit (Invitrogen) and APC Annexin V (Biolegend), and determining the cytotoxicity on the flow cytometry (iQue Screener). Controls consisted of cells treated as above except that no primary antibody was added, and cells treated as above except an irrelevant isotype-matched control antibody was used instead of a test antibody. FIGS. 13A and B show the results of the ADCC assay using PBMCs from two different donors.

### Example 14: In Vivo Xenograft Murine Model (RPMI8226)

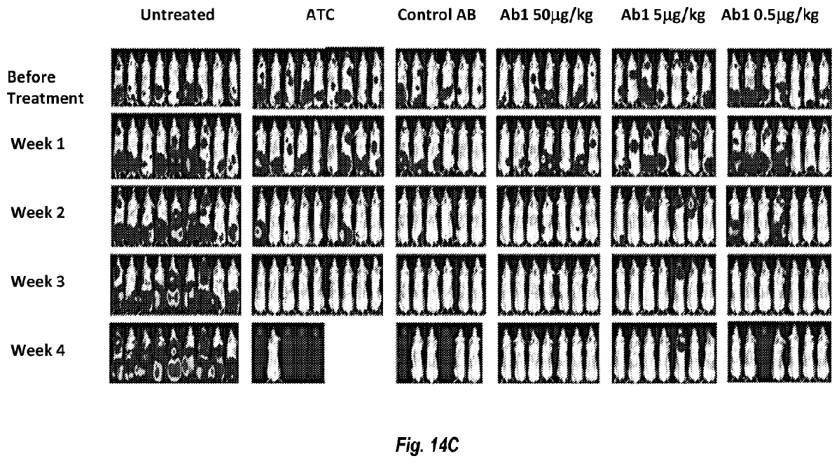

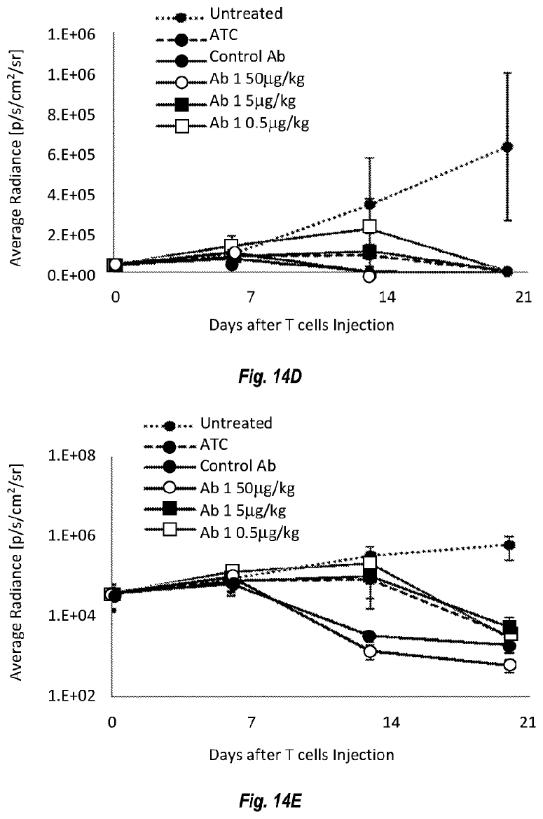

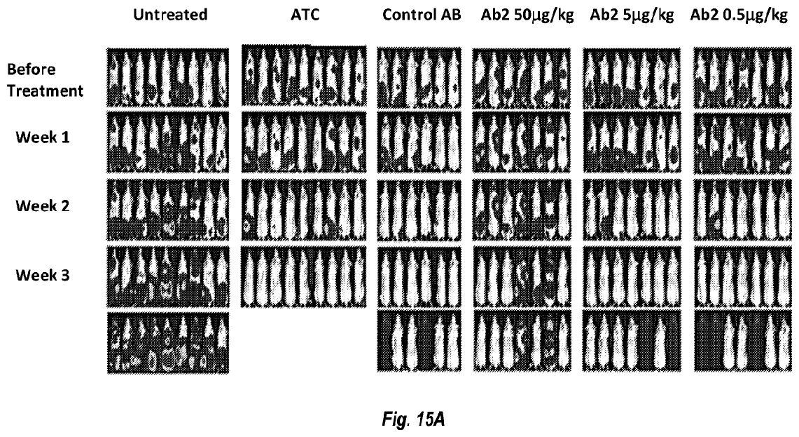

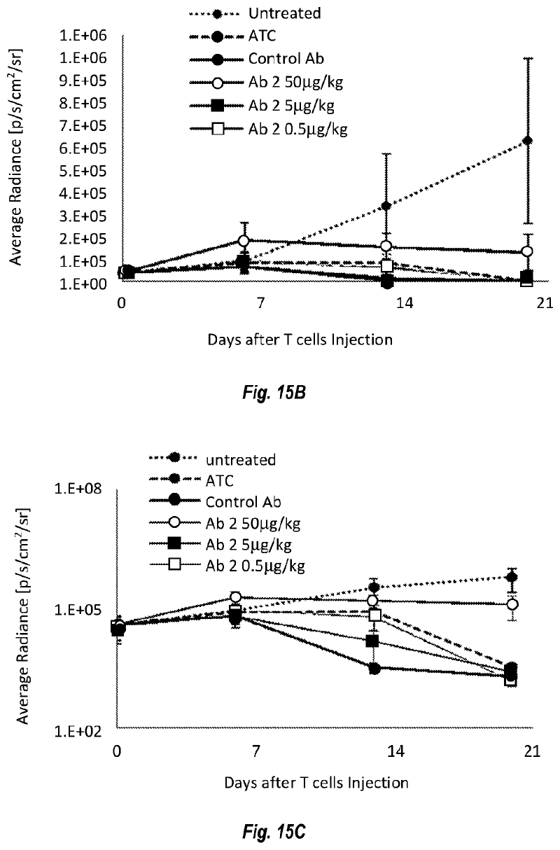

An in vivo xenograft murine model was used to measure the tumoricidal activity (RPMI8226) of a heterodimeric (bispecific) antibody in a bioluminescent xenograft animal model. The treatment schedule is shown in FIG. 14A and treatment cohorts are listed in the Table in FIG. 14B. The negative control mice were administered phosphate-buffered saline. Control mice were administered either activated T cells or a control RSV/CD3 bispecific antibody. The test mice were administered either 50 ug/kg, 5 ug/kg or 0.5 ug/kg of a CD38/CD3 bispecific antibody (Ab1) (FIGS. 14C and 15A). The results shown in FIG. 14C indicate that the CD38/CD3 bispecific antibody (Ab1) has anti-tumor efficacy and delays GVHD symptoms. The results shown in FIG. 15A indicate that the CD38/CD3 bispecific antibody (Ab2) does not have anti-tumor efficacy. The average bioluminescence signals from the treated mice is shown in FIGS. 14D-E (for Ab1) and FIGS. 15B-C (for Ab2).

FIG. 15A shows bioluminescent signals from the mouse xenograft model described in the schedule shown in FIGS. 14A and B. These mice were treated with a different CD38/CD3 bispecific antibody (Ab2) compared to the mice shown in FIG. 14C. FIGS. 15B-C show the average bioluminescence signals from the treated mice is shown in FIG. 15A.

### Example 15: In Vivo Xenograft Murine Model (RPMI8226 and T Cells)

Active T cells: Healthy donor blood was obtained from San Diego blood bank and PBMCs (Donor 8) were isolated with Histo-paque via density gradient centrifuge technique. PBMCs were activated by OKT3 50 ng/ml and cultured until injection.

RPMI 82226-GFP-Fluc cells: Human multiple myeloma cell line RPMI 8226 obtained from ATCC (CCL-155), and the retroviral vector PMY-MA005, yielding GFP and firefly luciferase genes was transduced. By limiting dilution, single cell clone was selected and a stable RPMI 8226-GFP-Fluc cell line was generated. The RPMI 8226-GFP-Fluc cell line was cultured in RPMI1640+10% FBS medium for xenogeneic mouse transplant multiple myeloma model. 8 week old female NSG immunodeficient mice were use (from Jackson Laboratories)

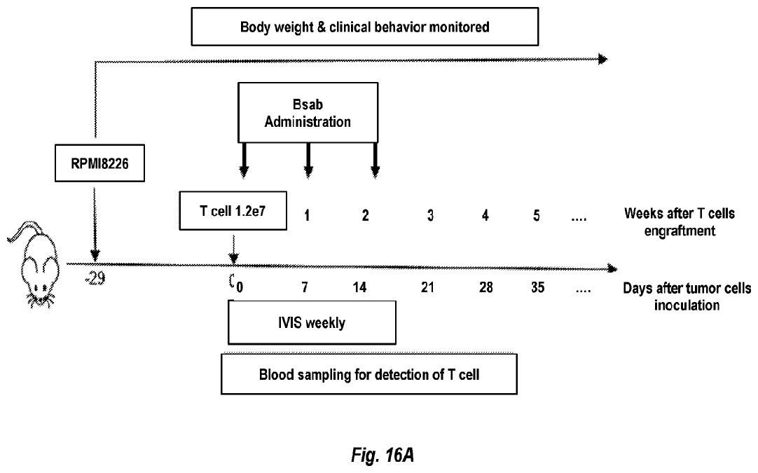

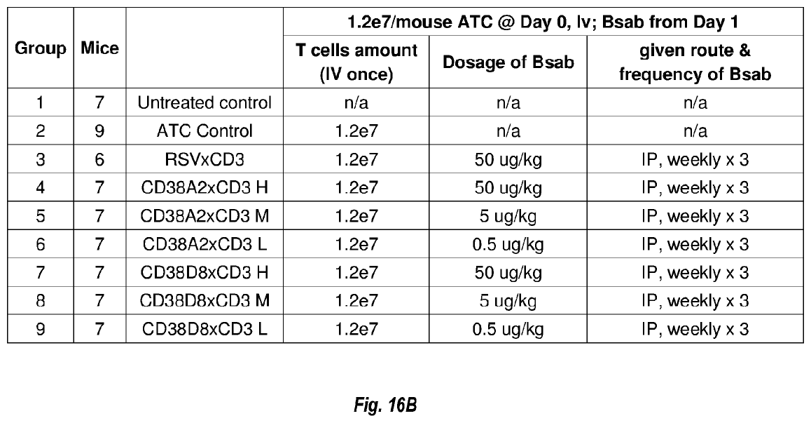

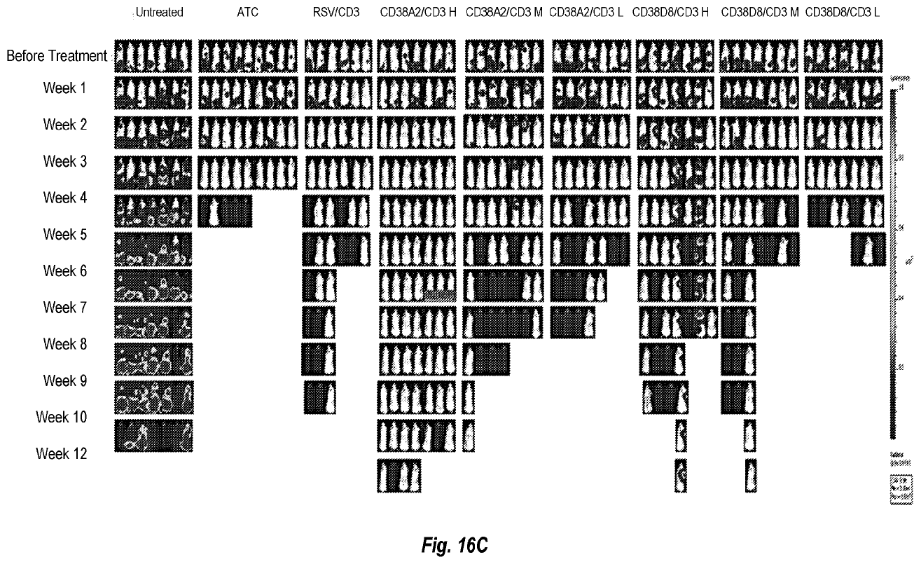

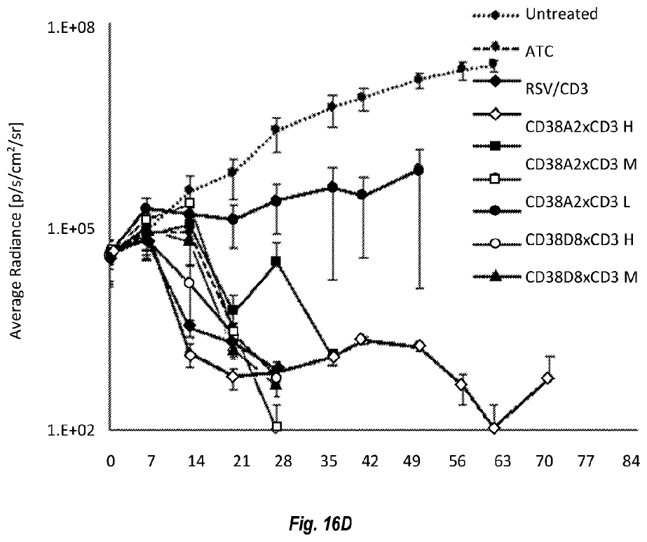

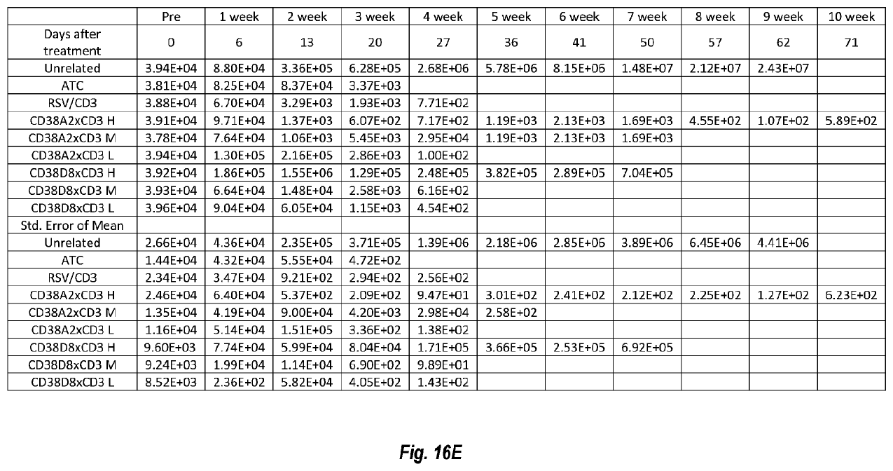

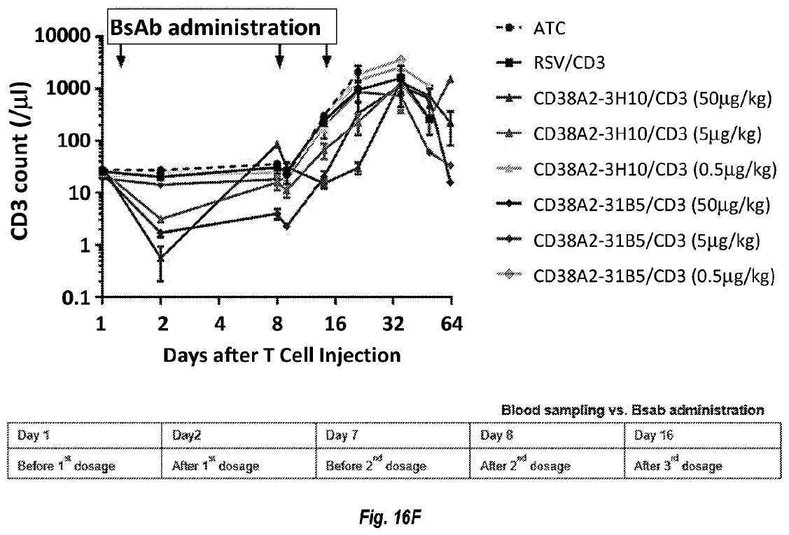

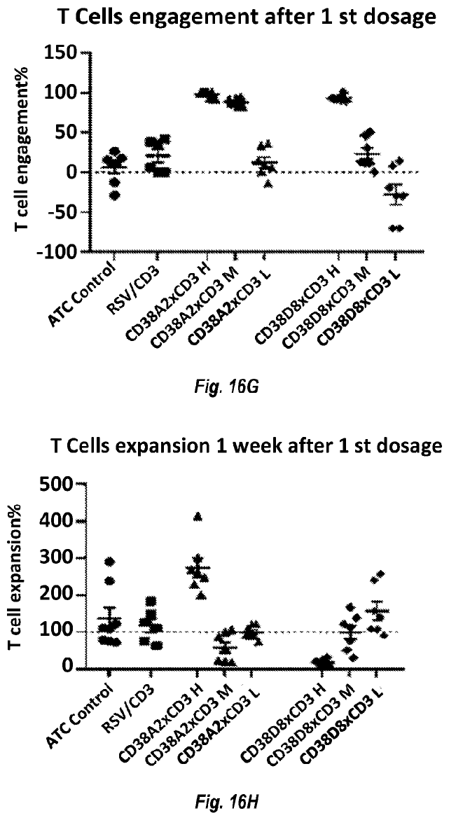

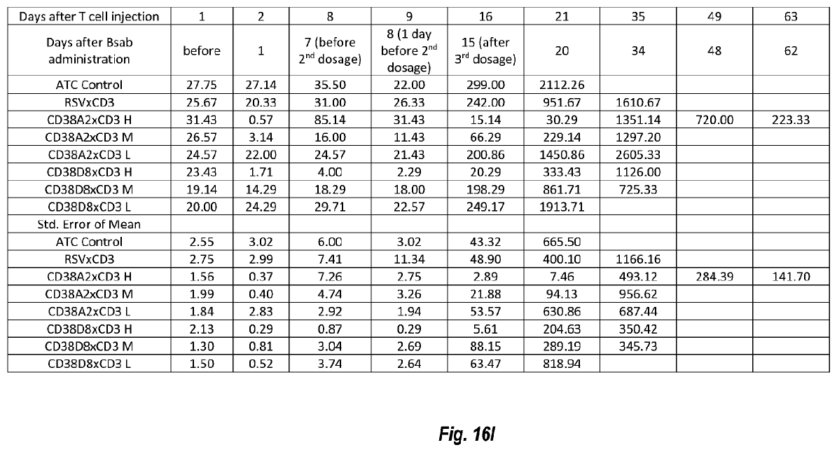

Systemic CD38+ human multiple myeloma xenograft Animal model with RPMI 8226-GFP-Fluc cells: The RPMI 8226-GFP-Fluc cells were cultured in RPMI-1640 medium supplemented with 10% fetal bovine serum in culture flasks. Xenogeneic MM was generated by intravenous inoculation of 1e7 RPMI8226-GFP-Fluc cells via the tail vein into unconditioned adult NSG. 1.2e7 of active T cells were injected in the animals via tail vein in a volume of 0.2 mL/mouse 29 days after tumor inoculation when the tumor could be detected with IVIS (FIGS. 16A-B). The testing bispecific antibodies and control antibody were given accordingly the day after ATC injection. The mice in the untreated group were not received ATC or any treatment. Tumor burden was evaluated weekly by IVIS.

Tumor Burden Evaluation by IVIS: IVIS is the imaging system using multispectral fluorescence and bioluminescence together. The mice in this experiment were evaluated for tumor burdens via luminescence using IVIS imaging system every week. General anesthesia was initiated using Patterson Veterinary's anesthesia instrument and Luciferin was administrated to the mouse at 150 mg/kg via intraperitoneal injection. The injected mice were placed in the Isoflurane induction chamber for 5 minutes before acquisition of bioluminescence signals. The imaging procedure and data analysis were performed following the IVIS manufacturer's instructions. The bioluminescent signal flux for each mouse as photons/s per cm² per steradian was analyzed and color scale was set at min=1e5 and max=1e7 by the Living Image software (PerkinElmer). IVIS imaging of the animals is shown in FIG. 16C and the graph of average radiance is shown in FIG. 16D.

Engraftment of T cells in mice: Peripheral blood was obtained via tail vein bleeding (see Tables shown in FIGS. 16E and 16I). The samples were examined for engraftment of human lymphocytes by staining with APC-anti-CD3. The final CD3+ cell count per microliter was calculated with dilution factor 20. In brief, whole blood was incubated with the relevant antibodies for 1 hour at room temperature in the dark, and erythrocytes were subsequently removed by Fixative-Free Lysing Solution (Life Technologies). Samples were analyzed using the Attune Nxt Flow Cytometer (Life Technologies) as described in the manufacturer's instructions.

Formula and calculation: The Attune Nxt Flow Cytometer was used to record the cell concentration as cell count per microliter. The blood sample was diluted 20-fold and the real concentration was the measured concentration multiplied by the dilution fold of 20.

Statistical analysis: Statistical analyses were performed using Microsoft Excel and GraphPad Prism version 7.0 (GraphPad Software). All measures of variance were presented as standard error of the mean (SEM). Results were assessed for normal Gaussian distribution and then analyzed by Log-Rank (Mantel-Cox) test as indicated. Data were significantly different when P≤0.05, ≤0.01, or ≤0.001 represented in the figures and tables as *, **, or ***, respectively.

The results in FIGS. 16F-I show that the CD38×CD3 bispecific antibody could significantly engage over 90% of T cell one day after bispecific antibody administration, which results in 3-fold expansion of T cell 7 days after antibody administration.

### Example 16: In Vivo Xenograft Murine Model (Raji and PBMCs)

This in vivo study was designed to examine the antitumor efficacy of anti-CD3/CD38 bispecific antibody in systemic CD38+ human Burkitt's lymphoma xenograft animal model in NSG mice.

The NSG mice were inoculated with Raji-15 cells, which is CD38+ human B lymphocyte cell line, intravenously and mixed with thawed frozen PBMCs together, followed by treatment with DPBS or anti-CD3/RSV bispecific antibody or anti-CD3/CD38 bispecific antibody.

The bispecific antibody used in this study was: CD38A2-3H10m1/CD3tsm #1-scFvg1LALA-knob-in-hole. The control bispecific antibody was: RSV/CD3tsm #1-scFvg1LALA-knob-in-hole.

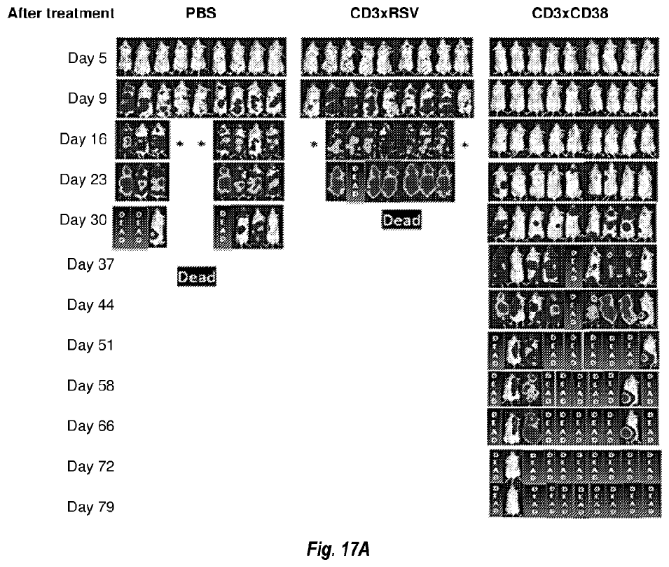

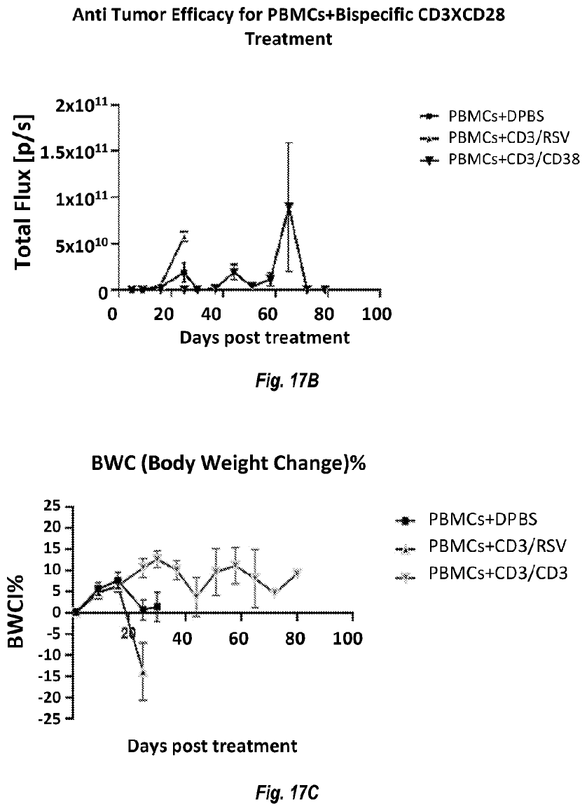

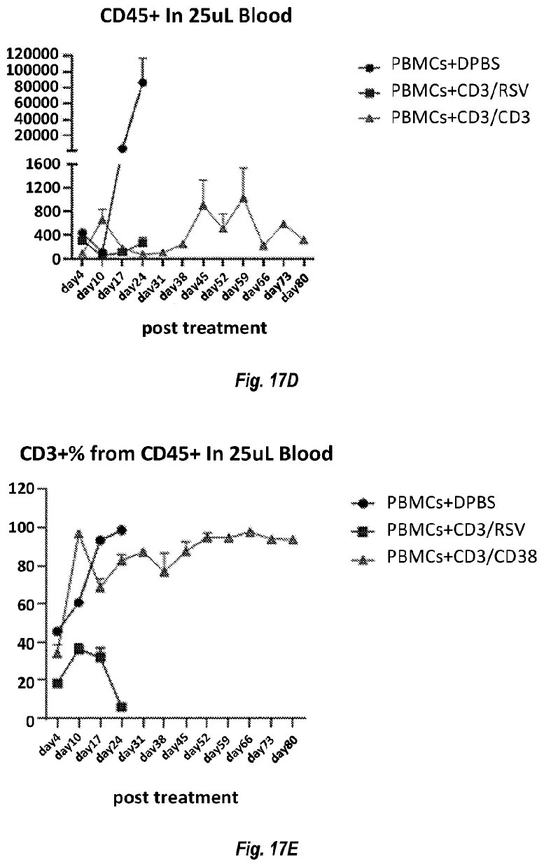

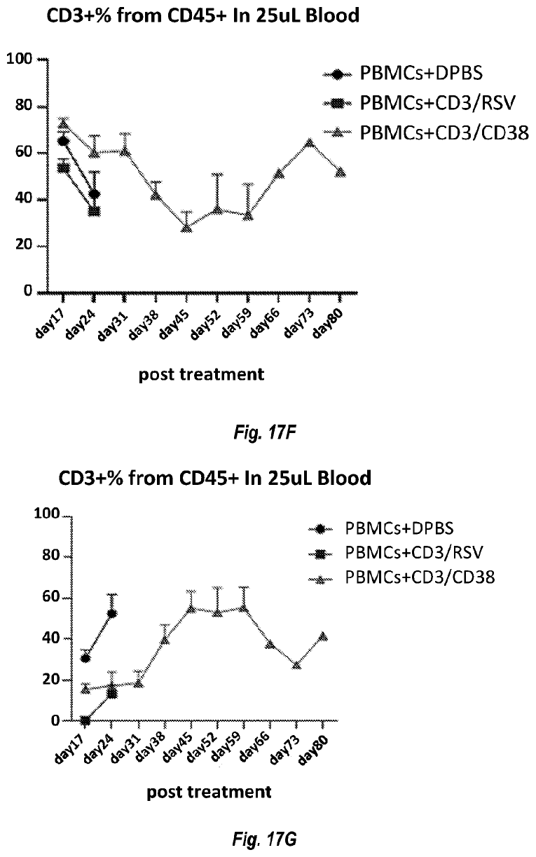

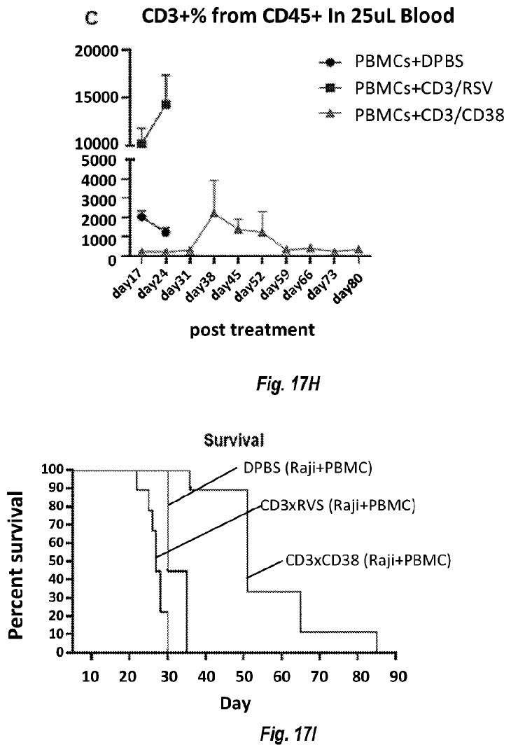

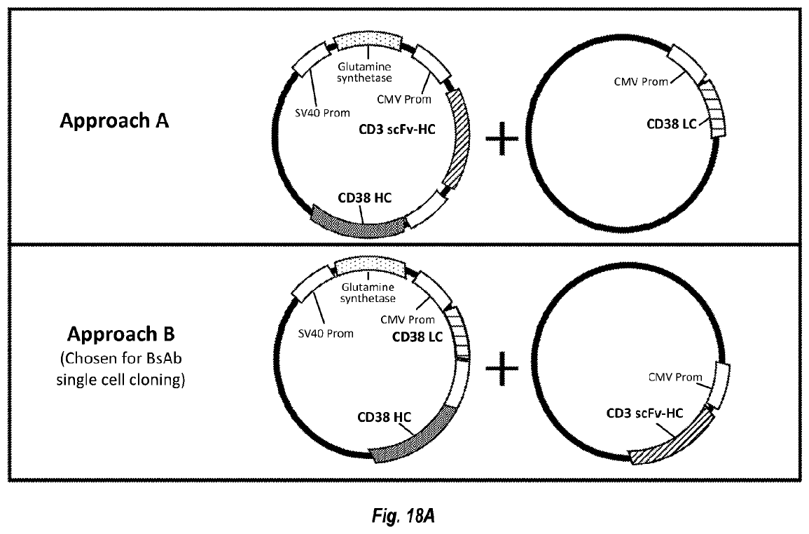

NSG mice were inoculated with 1×10⁵ Raji-15 cells and 5×10⁶ PBMCs. The animals (n=9) were treated with multiple doses (via intraperitoneal administration) of either 5 ug of anti-CD3/CD38 or control anti-CD3/RSV, or 200 uL DPBS. The animals were treated at 6 hours, 72 hours, 7 days, 10 days, 14 days, and 17 days (FIG. 17A). The body weight measurement and clinical observation were monitored regularly (FIG. 17D). If the animals became moribund or lost greater than 15% of their body weight due to the disease, they were euthanized. The tumor burden was monitored by IVIS weekly. Blood samples were taken via tail vein every week for the immune cells and tumor cells detection.

The engrafted human immune cells were stained with PE/Cy7 anti-CD3, APC/Cy7 anti-CD45, APC anti-CD38, PE anti-CD8a antibody, PerCP/Cyanine5.5 mouse anti-CD4 antibody and analyzed per 50 uL blood by flow cytometry.

The circulated CD45+ cell count (day 4 post treatment), CD3+% (day 4 post treatment), CD4+% (day 17 post treatment), and CD8+% immune cells (day 17 post treatment), were successfully detected. The total T cell counts (CD45+) were highest in mice that injected with PBMCs, followed by the PBMCs with control anti-CD3/RSV, and anti-CD3/CD38 had the least CD45+ number (FIG. 17E). CD3+% in total CD45+ population gradually increased along with the increase of CD45+ cells (FIG. 17F). CD4+% in total CD3+ population gradually decreased along with the increase of CD8+% cells (FIG. 17G). The percent CD8a+ in total CD3+ cells is shown in FIG. 17H. CD38 expression level in CD45+ population increased around 30 days after the treatment then dropped around 40 days (FIG. 17I).

The tumor burden in the mice treated with anti-CD3/CD38 bispecific antibody was eradicated until day 16 after the first treatment, and all nine mice survived after week 5 post treatment. In contrast, the tumor grew progressively in the mice that received DPBS treatment or received control anti-CD3/RSV bispecific antibody, until all the mice succumbed to tumors (survival days after T cells treatment as: DPBS group 32±2.64 days; anti-CD3/RSV group 27±2.5 days; anti-CD3/CD38 group 58±15.21 days). See FIGS. 17B and C. These results demonstrate that anti-CD3/CD38 bispecific antibody can effectively eradicate CD38-positive Burkitt's lymphoma and prolong the survival in vivo. (p<0.0001 by Logrank test). The survival graph is shown in FIG. 17J.

### Example 17: Stable Pool Production

Stable pool transfections were conducted using the LONZA GS Xceed™ Gene Expression System for CHOK1SV GS-KO cell lines which do not express glutamine synthetase. The GS Xceed System expression vector system carries the glutamine synthetase gene.

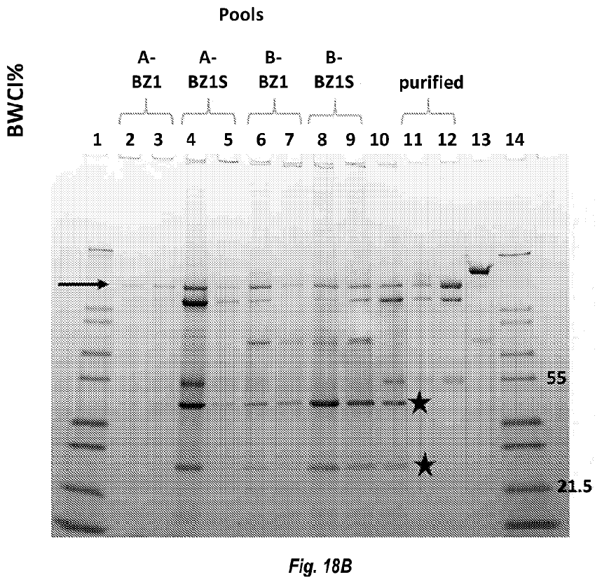

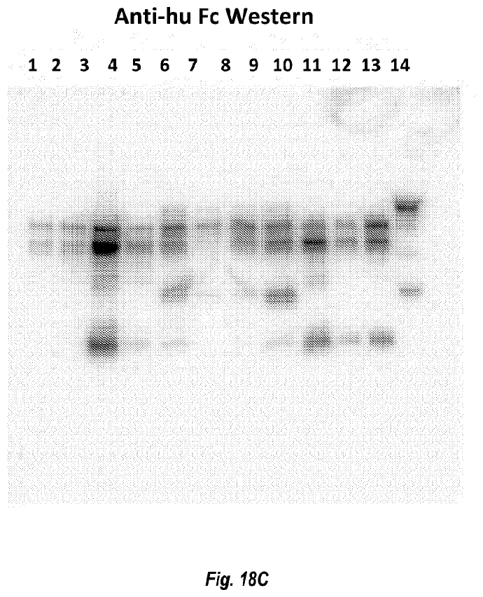

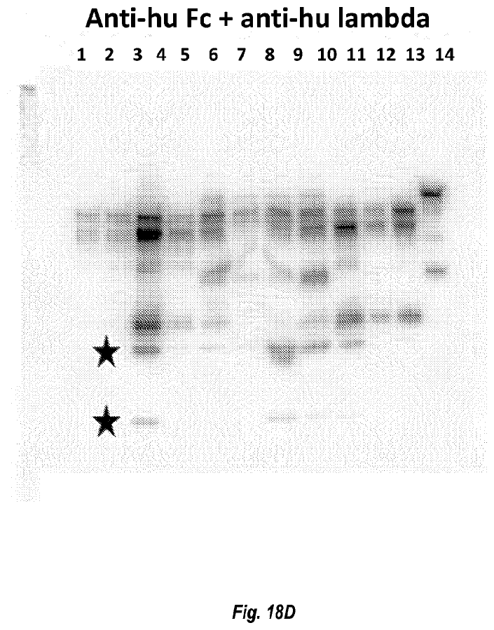

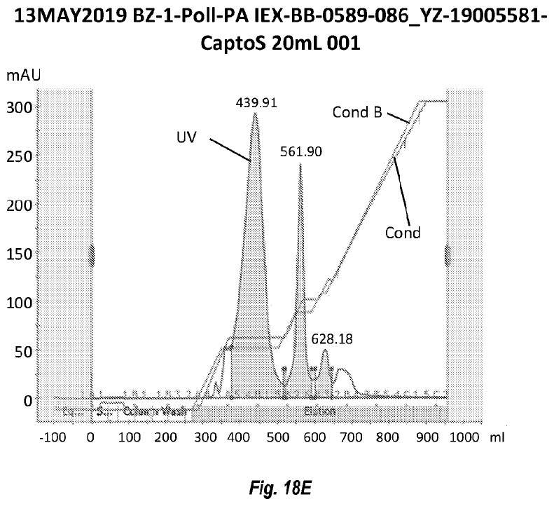

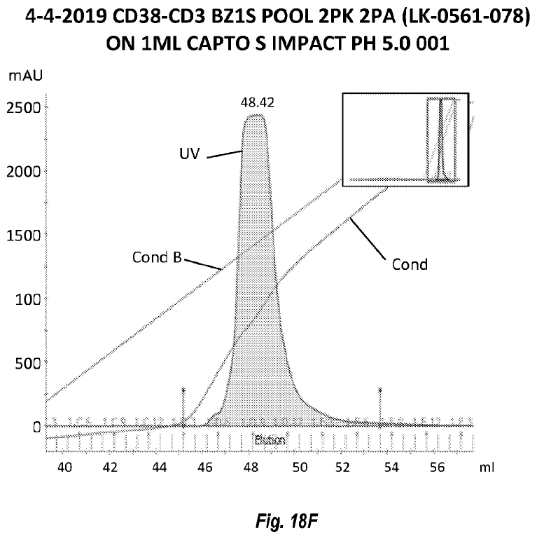

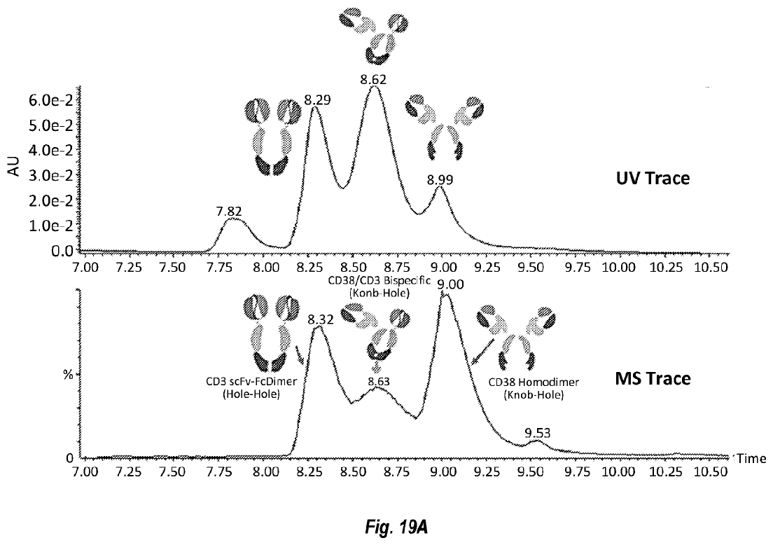

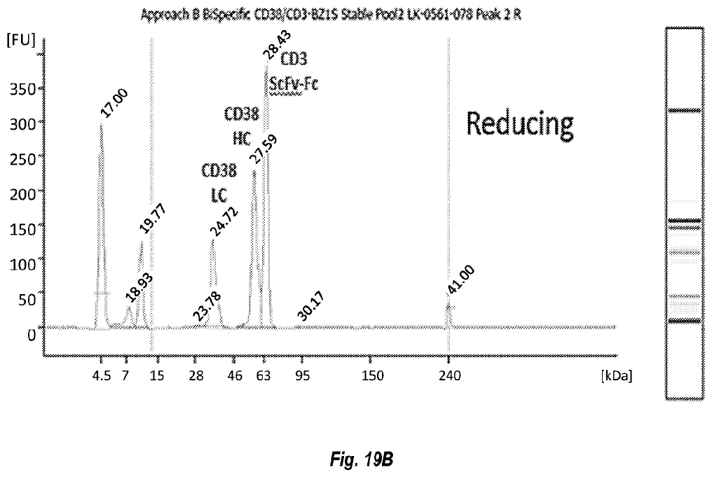

Two different cloning strategies were tested (see FIG. 18A). In approach A, a first DNA encoding the first polypeptide (CD3 scFv-HC) and a second DNA encoding the second polypeptide (CD38 HC) were cloned into a first expression vector that expresses glutamine synthetase, and a third DNA encoding the third polypeptide (CD38 LC) was cloned into a second expression vector. In approach B, a first DNA encoding the second polypeptide (CD38 HC) and a second DNA encoding the third polypeptide (CD38 LC) were cloned into a first expression vector that expresses glutamine synthetase, and a third DNA encoding the first polypeptide (CD3 scFv-HC) was cloned into a second expression vector.

The CHOK1SV GS-KO cells were transfected (according to manufacturer's instructions) with the LONZA expression vectors carrying DNA encoding the first, second and third polypeptides that comprise the heterodimeric antibodies described herein. L-methionine sulphoximine (MSX) was added to boost stringency selection. Standard cell selection, recovery and expansion were conducted, then the cells were cryopreserved, they were or grown, purified and analyzed. The cells were regularly monitored for viability for many weeks (e.g., >7 weeks). Viable cell density (VCD) and titer were also monitored. Titers were monitored using Octet.

In general, stable cell line and stable pool production included: gene synthesis and DGV construction (˜4 weeks); transfection and combine (˜2 days); selection and recovery (˜3-7 weeks); expansion (˜1-2 weeks); pool freezing (˜1 week); fed-batch (˜2 weeks); purification (˜1 week); and antibody analysis (˜1 week).

In general, single cell cloning process included: thawed pool (˜1-2 weeks); 5×96 well plates (˜1 day); imaged cells (˜2 weeks); tittered wells (˜2 days); expanded to 24-wells (˜1 week) and obtain titer and gel analyzed; expanded to 6-well (˜1 week) and obtained titer and gel analyzed; expanded and freeze (˜2-3 weeks); fed-batch (˜2 weeks); purification (˜2 weeks); analysis (˜2 weeks); top clone selection (˜1 week); RCBs at risk (˜6 weeks); top clones stability study (˜19 weeks).

The bispecific antibodies were isolated from the stable pool cells and analyzed by SDS-PAGE gel and Western blotting (FIGS. 18B, C and D).

The yield of the isolated bispecific antibodies (designated BZ1) was 150 mg/L, the recovery was 135 mg/L, and the % recovery was 90%. The isolated BZ1 bispecific antibody (112 mg) was loaded onto a 20 mL Capto™ S ImpAct column for purification. The results are shown in FIG. 18E. The final yield of the BZ1 bispecific antibody (isotype IgG1/LALA): concentration 3.64 mg/L; volume 12.0 mL; yield 43.68 mg; % recovery 39%; calculated PI 8.15; final buffer histidine pH 5.0.

The yield of the isolated bispecific antibodies (designated BZ1S) was 500 mg/L, the recovery was 343 mg/L, and the % recovery was 69%. The isolated BZ1S bispecific antibody (30 mg) was loaded onto a 1 mL Capto S ImpAct column for purification. The results are shown in FIG. 18F. The final yield of the BZ1S bispecific antibody (isotype IgG1/LALA): trans concentration 9.20 mg/L; volume 1.0 mL; % recovery 32%; calculated PI 8.15; final buffer histidine pH 5.0.

An LC-MS was conducted on the bispecific antibody and the UV and MS traced are shown in FIG. 18G. Schematics of the various homodimeric and heterodimeric antibody forms are included in the traces.

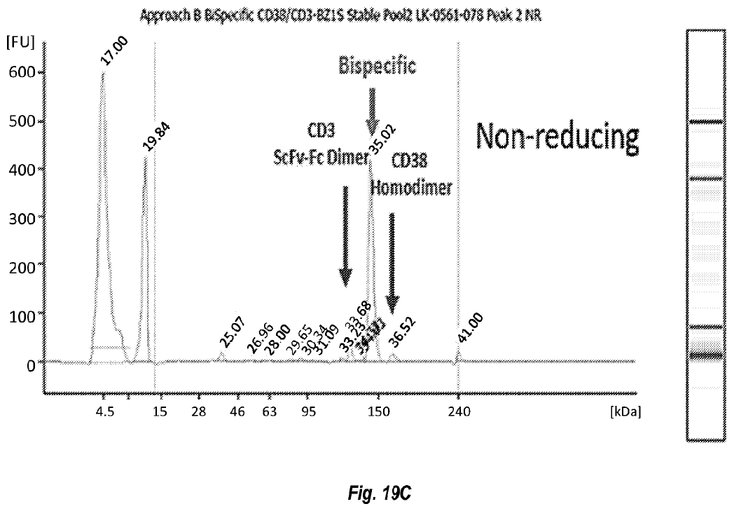

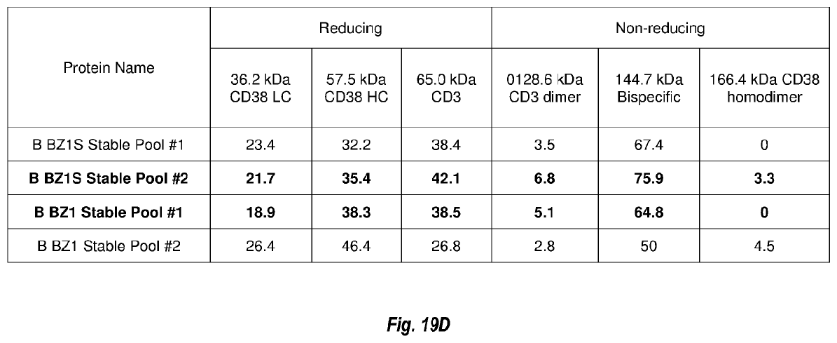

CE-SDS was conducted on the bispecific antibody under reducing and non-reducing conditions, and the results are shown in FIGS. 19B-C.

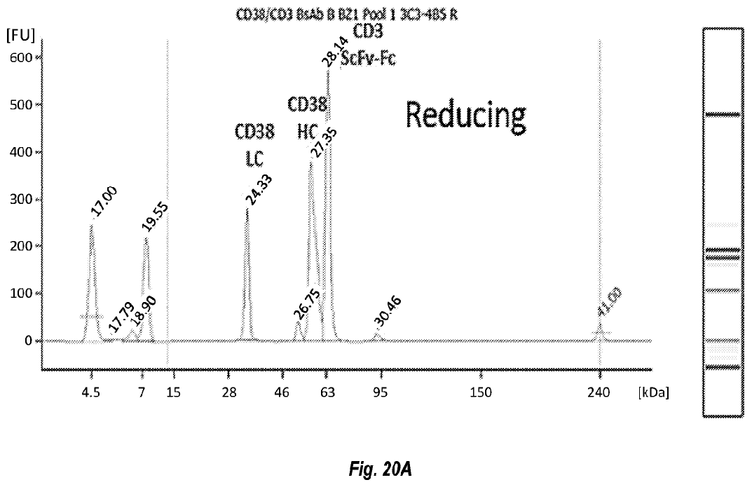

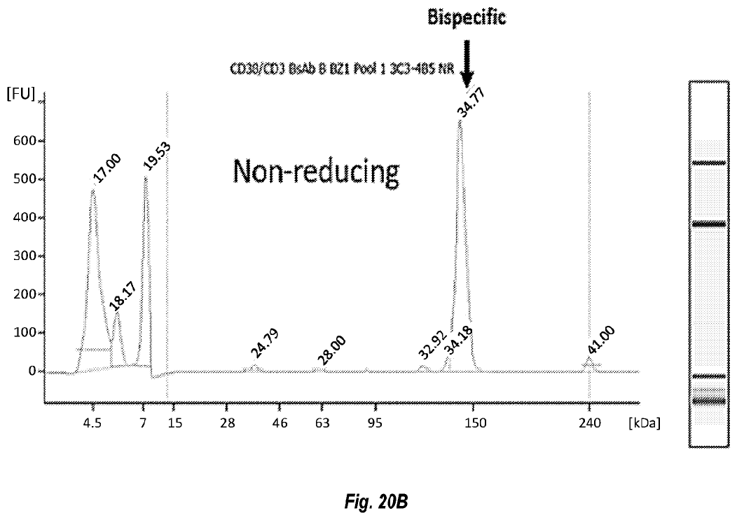

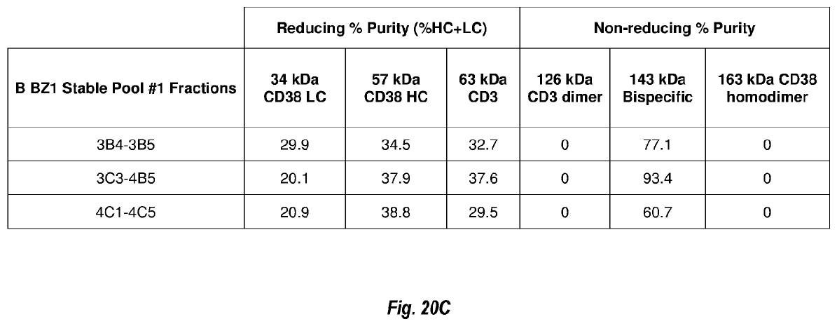

Ion exchange chromatography was conducted on the bispecific antibody BZ1 under reducing a non-reducing conditions, and the results are shown in FIGS. 20A-B.

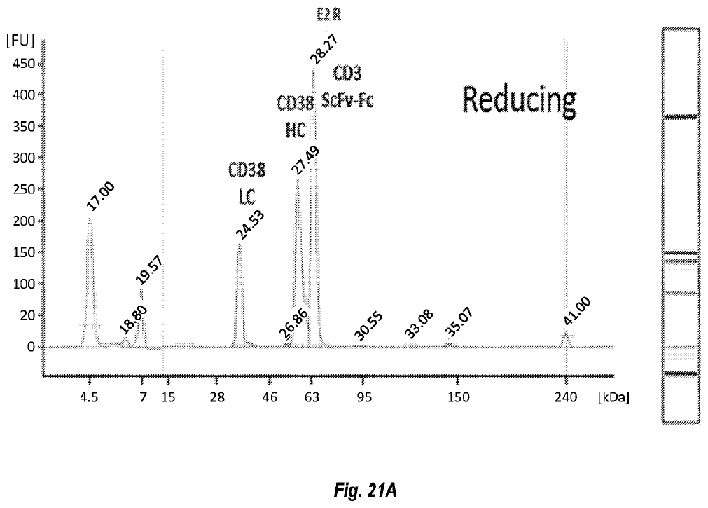

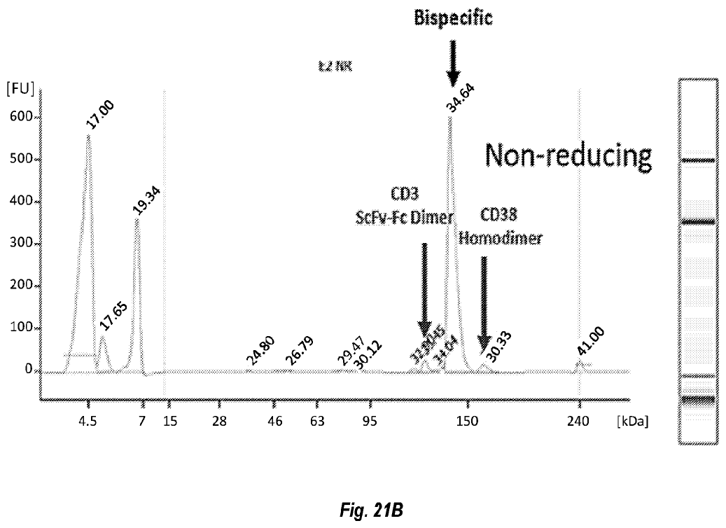

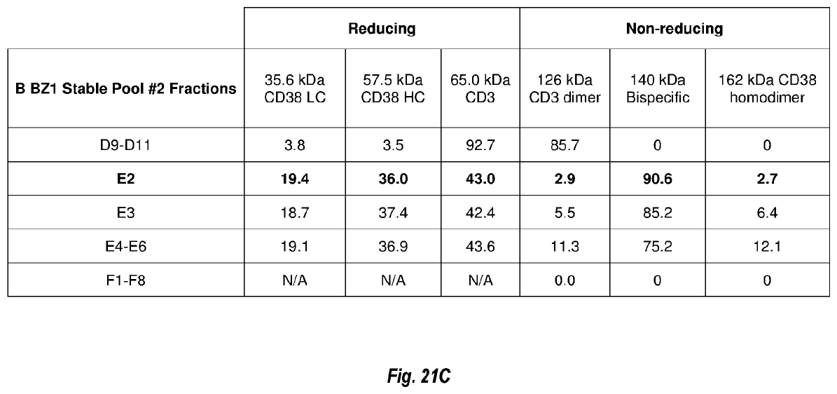

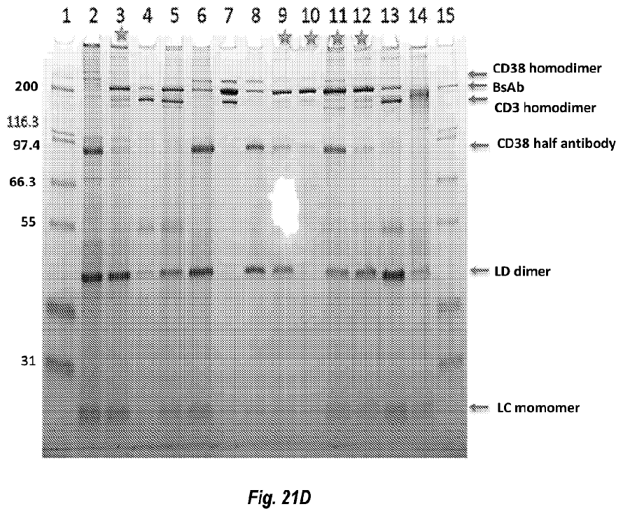

Ion exchange chromatography was conducted on the bispecific antibody BZ1S under reducing a non-reducing conditions, and the results are shown in FIGS. 21A-B.

What is claimed:

1. A heterodimeric antibody that binds a tumor-associated antigen and a T cell receptor, the antibody comprising:
a) a first polypeptide comprising an scFv and a first Fc region, wherein the scFv includes a first heavy chain variable region (VHa) and a first light chain variable region (VLa) joined together by a peptide linker, wherein the scFv is joined to the first Fc region by a first hinge region, and wherein the first heavy chain variable region (VHa) and a first light chain variable region (VLa) form a first antigen binding domain that binds the T cell receptor;
b) a second polypeptide comprising a second heavy chain variable region (VHb) and a second Fc region, wherein the second heavy chain variable region (VHb) is joined to the second Fc region by a second hinge region; and
c) a third polypeptide comprising a second light chain variable region (VLb), wherein the second heavy chain variable region (VHb) of the second polypeptide and the second light chain variable region (VLb) of the third polypeptide form a second antigen binding domain that binds the tumor-associated antigen, wherein the tumor-associated antigen comprises CD38, and
wherein the T cell receptor comprises CD3.

2. The heterodimeric antibody of claim 1, wherein
a) the first heavy chain variable region (VHa) of the first polypeptide comprises the CDR-H1, CDR-H2, and CDR-H3 sequences of a heavy chain variable region sequence selected from SEQ ID NOS:22, 28, 30, 32, 34, 36 and 38;
b) the first light chain variable region (VLa) of the first polypeptide comprises the CDR-L1, CDR-L2, and CDR-L3 sequences of a light chain variable region sequence selected from SEQ ID NOS:24, 29, 31, 33, 35, 37 and 39;
c) the second heavy chain variable region (VHb) of the second polypeptide comprises the CDR-H1, CDR-H2, and CDR-H3 sequences of a heavy chain variable region sequence selected from SEQ ID NOS:1, 6, 8, 10, 12, 14, 40, 42, 44, 46, 48, 50, 52, 54, 56, 58, 60, 62 and 64; and
d) the second light chain variable region (VLb) of the third polypeptide comprises the CDR-L1, CDR-L2, and CDR-L3 sequences of a light chain variable region sequence selected from SEQ ID NOS:16, 18, 20, 41, 43, 45, 47, 49, 51, 53, 55, 57, 59, 61, 63 and 65.

3. The heterodimeric antibody of claim 1 or 2, wherein
a) the first heavy chain variable region (VHa) of the first polypeptide comprises an amino acid sequence that is at least 95% identical to one of the sequences selected from SEQ ID NOS:22, 28, 30, 32, 34, 36 and 38;
b) the first light chain variable region (VLa) of the first polypeptide comprises an amino acid sequence that is at least 95% identical to one of the sequences selected from SEQ ID NOS:24, 29, 31, 33, 35, 37 and 39;
c) the second heavy chain variable region (VHb) of the second polypeptide comprises an amino acid sequence that is at least 95% identical to one of the sequences selected from SEQ ID NOS:1, 6, 8, 10, 12, 14, 40, 42, 44, 46, 48, 50, 52, 54, 56, 58, 60, 62 and 64; and
d) the second light chain variable region (VLb) of the third polypeptide comprises an amino acid sequence that is at least 95% identical to one of the sequences selected from SEQ ID NOS:16, 18, 20, 41, 43, 45, 47, 49, 51, 53, 55, 57, 59, 61, 63 and 65.

4. The heterodimeric antibody of claim 1 or 2, wherein
a) the first heavy chain variable region (VHa) of the first polypeptide comprises the amino acid sequence of any one of SEQ ID NOS:22, 28, 30, 32, 34, 36 and 38;
b) the first light chain variable region (VLa) of the first polypeptide comprises the amino acid sequence of any one of SEQ ID NOS:24, 29, 31, 33, 35, 37 and 39;
c) the second heavy chain variable region (VHb) of the second polypeptide comprises the amino acid sequence of any one of SEQ ID NOS:1, 6, 8, 10, 12, 14, 40, 42, 44, 46, 48, 50, 52, 54, 56, 58, 60, 62 and 64; and
d) the second light chain variable region (VLb) of the third polypeptide comprises the amino acid sequence of any one of SEQ ID NOS:16, 18, 20, 41, 43, 45, 47, 49, 51, 53, 55, 57, 59, 61, 63 and 65.

5. The heterodimeric antibody of any one of claims 1-4, wherein
a) first polypeptide comprises an amino acid sequence that is at least 95% identical to the amino acid sequence of SEQ ID NO:87, and a second polypeptide comprises an amino acid sequence that is at least 95% identical to the amino acid sequence of SEQ ID NO:89, and a third polypeptide comprises an amino acid sequence that is at least 95% identical to the amino acid sequence of SEQ ID NO:88 (e.g., BZ1);
b) the first polypeptide comprises an amino acid sequence that is at least 95% identical to the amino acid sequence of SEQ ID NO:90, and a second polypeptide comprises an amino acid sequence that is at least 95% identical to the amino acid sequence of SEQ ID NO:92, and a third polypeptide comprises an amino acid sequence that is at least 95% identical to the amino acid sequence of SEQ ID NO:91 (e.g., BZ1S);
c) the first polypeptide comprises an amino acid sequence that is at least 95% identical to the amino acid sequence of any one of SEQ ID NOs:71-77, and a second polypeptide comprises an amino acid sequence that is at least 95% identical to the amino acid sequence of SEQ ID NO:79, and a third polypeptide comprises an amino acid sequence that is at least 95% identical to the amino acid sequence of SEQ ID NO:85 (e.g., CD38 A2 series);
d) the first polypeptide comprises an amino acid sequence that is at least 95% identical to the amino acid sequence of any one of SEQ ID NOs:71-77, and a second polypeptide comprises an amino acid sequence that is at least 95% identical to the amino acid sequence of SEQ ID NO:78, and a third polypeptide comprises an amino acid sequence that is at least 95% identical to the amino acid sequence of SEQ ID NO:84 (e.g., CD38 A2-3H10m1 series);
e) the first polypeptide comprises an amino acid sequence that is at least 95% identical to the amino acid sequence of any one of SEQ ID NOs:71-77, and a second polypeptide comprises an amino acid sequence that is at least 95% identical to the amino acid sequence of any one of SEQ ID NOS:80-83, and a third polypeptide comprises an amino acid sequence that is at least 95% identical to the amino acid sequence of SEQ ID NO:86 (e.g., CD38 D8 series); or
f) the first polypeptide comprises an amino acid sequence that is at least 95% identical to the amino acid sequence of SEQ ID NO:100, and a second polypeptide comprises an amino acid sequence that is at least 95% identical to the amino acid sequence of SEQ ID NO:106, and a third polypeptide comprises an amino acid sequence that is at least 95% identical to the amino acid sequence of SEQ ID NO:86 (e.g., CD38 D8 bump-in-dent series).

6. The heterodimeric antibody of any one of the preceding claims, wherein the first and/or second Fc regions include a LALA mutation, and wherein the first and second Fc regions are mutated to form knob-in-hole or bump-into-dent structures.

7. The heterodimeric antibody of any one of the preceding claims, wherein the first Fc region of the first polypeptide comprises the amino acid sequences of SEQ ID NOS:26 and 27.

8. The heterodimeric antibody of any one of the preceding claims, wherein the second Fc region of the second polypeptide comprises the amino acid sequences of SEQ ID NOS:4 and 5.

9. One or more nucleic acids encoding the first, second, and third polypeptides of the heterodimeric antibody of any one of the preceding claims.

10. One or more vectors comprising the one or more nucleic acids of claim 9, optionally wherein the one or more vectors comprise a first and second vector, and
a) the first vector encodes the first and second polypeptides, and the second vector encodes the third polypeptide;
b) the first vector encodes the second and third polypeptides, and the second vector encodes the first polypeptide; or
c) the first vector encodes the first and third polypeptides, and the second vector encodes the second polypeptide.

11. A host cell, or a population of host cells, harboring the one or more nucleic acids of claim 9 or the one or more vectors of claim 10.

12. A method for preparing a heterodimeric antibody, comprising: culturing a population of host cells under conditions suitable for expressing the first, second and third polypeptides by the population of host cells, wherein individual host cells in the population of host cells harbor the one or more nucleic acids of claim 9 or the one or more vectors of claim 11, optionally wherein the one or more vectors comprise (i) a first vector encoding the first polypeptide and the second polypeptide and (ii) a second vector encoding the third polypeptide.

13. The method of claim 12, further comprising: isolating the first, second and third polypeptides from the population of cells.

14. The method of claim 12 or 13, further comprising: subjecting the first, second and third polypeptides to conditions suitable for associating the first, second and third polypeptides with each other to form the heterodimeric antibody.

15. A method for binding a tumor-associated antigen and a T cell receptor, comprising: contacting the heterodimeric antibody of any one of claims 1-8 with the tumor-associated antigen and the T cell receptor under conditions suitable for binding the heterodimeric antibody to the tumor-associated antigen and the T cell receptor.

16. The method of claim 15, wherein the tumor-associated antigen is expressed by a cell from a cancer of the prostate, breast, ovary, head and neck, bladder, skin, colorectal, anus, rectum, pancreas, lung (including non-small cell lung and small cell lung cancers), brain, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis, or testis, or from a leiomyoma, glioma, or glioblastoma.

17. The method of claim 15, wherein the tumor-associated antigen is expressed by a hematologic cancer.

18. The method of claim 17, wherein the hematologic cancer is a B chronic lymphocytic leukemia (B-CLL), B and T acute lymphocytic leukemia (LL), acute myeloid leukemia (AML), chronic lymphocytic leukemia (CLL), chronic myelogenous leukemia (CIVIL), hairy cell leukemia (HCL), myeloproliferative disorder/neoplasm (MPDS), myelodysplasia syndrome, non-Hodgkin's lymphoma (NHL) including Burkitt's lymphoma (BL), Waldenstrom's Macroglobulinemia, mantle cell lymphoma, AIDS-related lymphoma, Hodgkin's Lymphoma (HL), T cell lymphoma (TCL), multiple myeloma (MM), plasma cell myeloma, plamocytoma, giant cell myeloma, heavy-chain myeloma, or light chain or Bence-Jones myeloma.

19. The method of any one of claims 15-18, wherein the T cell receptor is expressed on an effector T cell and the tumor-associated antigen is expressed by a tumor or cancer cell.

20. The method of claim 19, wherein the effector T cell kills the tumor or cancer cell by mediating cytotoxic cell killing.

21. A method for treating a disease in a subject, comprising: administering to the subject a therapeutically effective amount of the heterodimeric antibody of any one of claims 1-8.

22. Use of the heterodimeric antibody of any one of claims 1-8 for the manufacture of a medicament for treating a disease.

23. The heterodimeric antibody of any one of claims 1-8, for use in treating a disease.

24. The method, use, or heterodimeric antibody for use of any one of claims 21-23, wherein the disease comprises a cancer of the prostate, breast, ovary, head and neck, bladder, skin, colorectal, anus, rectum, pancreas, lung (including non-small cell lung and small cell lung cancers), brain, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis, or testis, or from a leiomyoma, glioma, or glioblastoma.

25. The method, use, or heterodimeric antibody for use of any one of claims 21-23, wherein the disease comprises a hematologic cancer.

26. The method, use, or heterodimeric antibody for use of claim 25, wherein the hematologic cancer is B chronic lymphocytic leukemia (B-CLL), B and T acute lymphocytic leukemia (LL), acute myeloid leukemia (AML), chronic lymphocytic leukemia (CLL), chronic myelogenous leukemia (CML), hairy cell leukemia (HCL), myeloproliferative disorder/neoplasm (MPDS), myelodysplasia syndrome, non-Hodgkin's lymphoma (NHL) including Burkitt's lymphoma (BL), Waldenstrom's Macroglobulinemia, mantle cell lymphoma, AIDS-related lymphoma, Hodgkin's Lymphoma (HL), T cell lymphoma (TCL), multiple myeloma (MM), plasma cell myeloma, plamocytoma, giant cell myeloma, heavy-chain myeloma, or light chain or Bence-Jones myeloma.


SEQUENCE LISTING (131 sequences)

SEQ ID NO: 1 - protein, 118 aa, Artificial sequence
  2nd polypeptide  heavy chain - CD38A2-3H10m1g1LALA-K heavy chain variable sequence
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSS

SEQ ID NO: 2 - protein, 103 aa, Artificial sequence
  2nd polypeptide  heavy chain - CD38A2-3H10m1g1LALA-K heavy chain constant sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 3 - protein, 20 aa, Artificial sequence
  Hinge sequence
DKTHTCPPCPAPEAAGGPSV

SEQ ID NO: 4 - protein, 100 aa, Artificial sequence
  CH2 sequence
FLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 5 - protein, 107 aa, Artificial sequence
  CH3 sequence
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 118 aa, Artificial sequence
  2nd polypeptide - heavy chain - CD38A2g1LALA-K heavy chain variable sequence
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGQGTLVTVSS

SEQ ID NO: 7 - protein, 103 aa, Artificial sequence; identical to SEQ ID NO: 2
  2nd polypeptide - heavy chain - CD38A2g1LALA-K heavy chain constant sequence

SEQ ID NO: 8 - protein, 124 aa, Artificial sequence
  2nd polypeptide - heavy chain - CD38D8g1LALA-K heavy chain variable sequence
QVQLVESGGGVVQPGGSLRLSCAASGFIVSTNYVHWVRQAPGKGLEWVSGIYSDPYTSYAYSDSVKGRFTISRDMSKNTVYLQMNRLRAEDTAVYYCARETNTGFSNSWYLDFWGQGTLVTVSS

SEQ ID NO: 9 - protein, 103 aa, Artificial sequence; identical to SEQ ID NO: 2
  2nd polypeptide - heavy chain - CD38D8g1LALA-K heavy chain constant sequence

SEQ ID NO: 10 - protein, 124 aa, Artificial sequence
  2nd polypeptide - heavy chain - CD38D8-3A1g1LALA-K heavy chain variable sequence
QVQLVESGGGVVQPGGSLRLSCAASGFNVRTKYVHWVRQAPGKGLEWVSGIYSDPYTSYAYSDSVKGRFTISRDMSKNTVYLQMNRLRAEDTAVYYCARETNTGFSNSWYLDFWGQGTLVTVSS

SEQ ID NO: 11 - protein, 103 aa, Artificial sequence; identical to SEQ ID NO: 2
  2nd polypeptide - heavy chain - CD38D8-3A1g1LALA-K heavy chain constant sequence

SEQ ID NO: 12 - protein, 124 aa, Artificial sequence
  2nd polypeptide - heavy chain - CD38D8-3E1g1LALA-K heavy chain variable sequence
QVQLVESGGGVVQPGGSLRLSCAASKLNVSPKYVHWVRQAPGKGLEWVSGIYSDPYTSYAYSDSVKGRFTISRDMSKNTVYLQMNRLRAEDTAVYYCARETNTGFSNSWYLDFWGQGTLVTVSS

SEQ ID NO: 13 - protein, 103 aa, Artificial sequence; identical to SEQ ID NO: 2
  2nd polypeptide - heavy chain - CD38D8-3E1g1LALA-K heavy chain constant sequence

SEQ ID NO: 14 - protein, 124 aa, Artificial sequence
  2nd polypeptide - heavy chain - CD38D8-1B5g1LALA-K heavy chain variable sequence
QVQLVESGGGVVQPGGSLRLSCAASGLNARTNYVHWVRQAPGKGLEWVSGIYSDPYTSYAYSDSVKGRFTISRDMSKNTVYLQMNRLRAEDTAVYYCARETNTGFSNSWYLDFWGQGTLVTVSS

SEQ ID NO: 15 - protein, 103 aa, Artificial sequence; identical to SEQ ID NO: 2
  2nd polypeptide - heavy chain - CD38D8-1B5g1LALA-K heavy chain constant sequence

SEQ ID NO: 16 - protein, 110 aa, Artificial sequence
  3rd polypeptide  light chain - CD38A2-3H10m1 LC light chain variable sequence
QSALTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 17 - protein, 106 aa, Artificial sequence
  3rd polypeptide  light chain - CD38A2-3H10m1 LC light chain constant sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 110 aa, Artificial sequence
  3rd polypeptide  light chain - CD38A2 LC light chain variable sequence
QSALTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL

SEQ ID NO: 19 - protein, 106 aa, Artificial sequence; identical to SEQ ID NO: 17
  3rd polypeptide  light chain - CD38A2 LC light chain constant sequence

SEQ ID NO: 20 - protein, 110 aa, Artificial sequence
  3rd polypeptide  light chain - CD38D8 LC light chain variable sequence
QPVLTQPPSASGTPGQRVTISCSGSSSNIGRNIVNWYQQLPGTTPKLLIYSNNQRPSGVPDRFSGSKSGTSASLAISGLHSEDEADYYCATWDDSLNGWVFGGGTKLTVL

SEQ ID NO: 21 - protein, 106 aa, Artificial sequence; identical to SEQ ID NO: 17
  3rd polypeptide  light chain - CD38D8 LC light chain constant sequence

SEQ ID NO: 22 - protein, 120 aa, Artificial sequence
  1st polypeptide  scFv-Fc fusion - CD3-tsm#1 (M48I/K67R)  heavy chain variable sequence
EVQLVQSGAEVKKPGASVKVSCKASGYTFISYTMHWVRQAPGQGLEWIGYINPRSGYTHYNQKLKDRATLTADKSASTAYMELSSLRSEDTAVYYCARSAYYDYDGFAYWGQGTLVTVSS

SEQ ID NO: 23 - protein, 20 aa, Artificial sequence
  1st polypeptide  scFv-Fc fusion - linker sequence
GGGGSGGGGSGGGGSGGGGS

SEQ ID NO: 24 - protein, 106 aa, Artificial sequence [sequence omitted]

SEQ ID NO: 25 - protein, 25 aa, Artificial sequence
  1st polypeptide  scFv-Fc fusion - hinge sequence
GGSGGDKTHTCPPCPAPEAAGGPSV

SEQ ID NO: 26 - protein, 100 aa, Artificial sequence; identical to SEQ ID NO: 4
  1st polypeptide  scFv-Fc fusion - CH2 sequence

SEQ ID NO: 27 - protein, 107 aa, Artificial sequence
  1st polypeptide  scFv-Fc fusion - CH3 sequence
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 120 aa, Artificial sequence
  1st polypeptide  scFv-Fc fusion - CD3-tsm#2 (I30V/K67R)  heavy chain variable sequence
EVQLVQSGAEVKKPGASVKVSCKASGYTFVSYTMHWVRQAPGQGLEWMGYINPRSGYTHYNQKLKDRATLTADKSASTAYMELSSLRSEDTAVYYCARSAYYDYDGFAYWGQGTLVTVSS

SEQ ID NO: 29 - protein, 106 aa, Artificial sequence [sequence omitted]

SEQ ID NO: 30 - protein, 120 aa, Artificial sequence
  1st polypeptide  scFv-Fc fusion - CD3-tsm#3 (M48I)  heavy chain variable sequence
EVQLVQSGAEVKKPGASVKVSCKASGYTFISYTMHWVRQAPGQGLEWIGYINPRSGYTHYNQKLKDKATLTADKSASTAYMELSSLRSEDTAVYYCARSAYYDYDGFAYWGQGTLVTVSS

SEQ ID NO: 31 - protein, 106 aa, Artificial sequence [sequence omitted]

SEQ ID NO: 32 - protein, 120 aa, Artificial sequence
  1st polypeptide  scFv-Fc fusion - CD3-tsm#4 (I30V/M48I/K67R) heavy chain variable sequence
EVQLVQSGAEVKKPGASVKVSCKASGYTFVSYTMHWVRQAPGQGLEWIGYINPRSGYTHYNQKLKDRATLTADKSASTAYMELSSLRSEDTAVYYCARSAYYDYDGFAYWGQGTLVTVSS

SEQ ID NO: 33 - protein, 106 aa, Artificial sequence [sequence omitted]

SEQ ID NO: 34 - protein, 120 aa, Artificial sequence
  1st polypeptide  scFv-Fc fusion - CD3-tsm#5 (K67R)  heavy chain variable sequence
EVQLVQSGAEVKKPGASVKVSCKASGYTFISYTMHWVRQAPGQGLEWMGYINPRSGYTHYNQKLKDRATLTADKSASTAYMELSSLRSEDTAVYYCARSAYYDYDGFAYWGQGTLVTVSS

SEQ ID NO: 35 - protein, 106 aa, Artificial sequence [sequence omitted]

SEQ ID NO: 36 - protein, 120 aa, Artificial sequence
  1st polypeptide  scFv-Fc fusion - CD3-tsm#6 ( (I30V)  heavy chain variable sequence
EVQLVQSGAEVKKPGASVKVSCKASGYTFVSYTMHWVRQAPGQGLEWMGYINPRSGYTHYNQKLKDKATLTADKSASTAYMELSSLRSEDTAVYYCARSAYYDYDGFAYWGQGTLVTVSS

SEQ ID NO: 37 - protein, 106 aa, Artificial sequence [sequence omitted]

SEQ ID NO: 38 - protein, 120 aa, Artificial sequence
  1st polypeptide  scFv-Fc fusion - CD3-tsm#7 (WT)  heavy chain variable sequence
EVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSS

SEQ ID NO: 39 - protein, 106 aa, Artificial sequence [sequence omitted]

SEQ ID NO: 40 - protein, 118 aa, Artificial sequence
  Anti-CD38 A2-SV variant  heavy chain variable region - CD38-A2 wild type
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGRGTLVTVSS

SEQ ID NO: 41 - protein, 110 aa, Artificial sequence
  Anti-CD38 A2-SV variant  light chain variable region - CD38-A2 wild type
QAGLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL

SEQ ID NO: 42 - protein, 118 aa, Artificial sequence; identical to SEQ ID NO: 40
  Anti-CD38 A2-SV variant  heavy chain variable region - CD38-A2 SV mutant

SEQ ID NO: 43 - protein, 110 aa, Artificial sequence
  Anti-CD38 A2-SV variant  light chain variable region - CD38-A2 SV mutant
QSVLTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVL

SEQ ID NO: 44 - protein, 118 aa, Artificial sequence; identical to SEQ ID NO: 40
  Anti-CD38 3H10 variant  heavy chain variable region - A2 parental heavy chain

SEQ ID NO: 45 - protein, 110 aa, Artificial sequence; identical to SEQ ID NO: 41
  Anti-CD38 3H10 variant  light chain variable region - A2 parental light chain

SEQ ID NO: 46 - protein, 118 aa, Artificial sequence; identical to SEQ ID NO: 1
  Anti-CD38 3H10 variant  heavy chain variable region - 3H10m1

SEQ ID NO: 47 - protein, 110 aa, Artificial sequence
  Anti-CD38 3H10 variant  light chain variable region - 3H10m1
QAGLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 48 - protein, 118 aa, Artificial sequence
  Anti-CD38 3H10 variant  heavy chain variable region - 3G8m1
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTNWGQGTLVTVSS

SEQ ID NO: 49 - protein, 110 aa, Artificial sequence; identical to SEQ ID NO: 47
  Anti-CD38 3H10 variant  light chain variable region - 3G8m1

SEQ ID NO: 50 - protein, 118 aa, Artificial sequence
  Anti-CD38 3H10 variant  heavy chain variable region  3E3m1
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWGGEFTDWGQGTLVTVSS

SEQ ID NO: 51 - protein, 110 aa, Artificial sequence; identical to SEQ ID NO: 47
  Anti-CD38 3H10 variant  light chain variable region  3D3m1

SEQ ID NO: 52 - protein, 118 aa, Artificial sequence
  Anti-CD38 3H10 variant  heavy chain variable region  3G3
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREAWSGEFTDWGQGTLVTVSS

SEQ ID NO: 53 - protein, 110 aa, Artificial sequence; identical to SEQ ID NO: 41
  Anti-CD38 3H10 variant  light chain variable region  3G3

SEQ ID NO: 54 - protein, 118 aa, Artificial sequence
  Anti-CD38 3H10 variant  heavy chain variable region  3E11
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWGGEFTDWGQGTLVTVSS

SEQ ID NO: 55 - protein, 110 aa, Artificial sequence; identical to SEQ ID NO: 41
  Anti-CD38 3H10 variant  light chain variable region  3E11

SEQ ID NO: 56 - protein, 118 aa, Artificial sequence; identical to SEQ ID NO: 1
  Anti-CD38 3H10 variant  heavy chain variable region  3H10

SEQ ID NO: 57 - protein, 110 aa, Artificial sequence; identical to SEQ ID NO: 41
  Anti-CD38 3H10 variant  light chain variable region  3H10

SEQ ID NO: 58 - protein, 118 aa, Artificial sequence; identical to SEQ ID NO: 6
  Anti-CD38 3H10 variant  heavy chain variable region  3H10N

SEQ ID NO: 59 - protein, 110 aa, Artificial sequence
  Anti-CD38 3H10 variant  light chain variable region  3H10N
QSVLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 60 - protein, 118 aa, Artificial sequence
  Anti-CD38 3H10 variant  heavy chain variable region  3H10NS
QVQLVESGGGLVKPGGSLRLSCAASGFTFSDDYMSWIRQAPGKGLEWVASVSSGRPTTYYADSVRGRFTISRDNAKNSLYLQMNSLRAEDTAVYYCAREDWGGEFTDWGQGTLVTVSS

SEQ ID NO: 61 - protein, 110 aa, Artificial sequence; identical to SEQ ID NO: 59
  Anti-CD38 3H10 variant  light chain variable region  3H10NS

SEQ ID NO: 62 - protein, 118 aa, Artificial sequence; identical to SEQ ID NO: 40
  Anti-CD38 3H10 variant  heavy chain variable region  3E10

SEQ ID NO: 63 - protein, 110 aa, Artificial sequence; identical to SEQ ID NO: 47
  Anti-CD38 3H10 variant  light chain variable region  3E10

SEQ ID NO: 64 - protein, 118 aa, Artificial sequence; identical to SEQ ID NO: 1
  Anti-CD38 3H10 variant  heavy chain variable region  3H10m1g

SEQ ID NO: 65 - protein, 110 aa, Artificial sequence; identical to SEQ ID NO: 59
  Anti-CD38 3H10 variant  light chain variable region  3H10m1g

SEQ ID NO: 66 - protein, 330 aa, Artificial sequence
  Anti-CD38 IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 330 aa, Artificial sequence
  Anti-CD38 IgG1-SPPC heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 327 aa, Artificial sequence
  Anti-CD38 IgG4 heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - protein, 107 aa, Artificial sequence [sequence omitted]

SEQ ID NO: 70 - protein, 258 aa, Artificial sequence
  Antigen  CD38 protein
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 71 - protein, 478 aa, Artificial sequence
  1st polypeptide  scFv-Fc fusion - CD3-tsm#1 (M48I/K67R)  full length sequence
EVQLVQSGAEVKKPGASVKVSCKASGYTFISYTMHWVRQAPGQGLEWIGYINPRSGYTHYNQKLKDRATLTADKSASTAYMELSSLRSEDTAVYYCARSAYYDYDGFAYWGQGTLVTVSSGGGGSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 72 - protein, 478 aa, Artificial sequence
  1st polypeptide  scFv-Fc fusion - CD3-tsm#2 (I30V/K67R)  full length sequence
EVQLVQSGAEVKKPGASVKVSCKASGYTFVSYTMHWVRQAPGQGLEWMGYINPRSGYTHYNQKLKDRATLTADKSASTAYMELSSLRSEDTAVYYCARSAYYDYDGFAYWGQGTLVTVSSGGGGSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 73 - protein, 478 aa, Artificial sequence
  1st polypeptide  scFv-Fc fusion - CD3-tsm#3 (M48I)  full length sequence
EVQLVQSGAEVKKPGASVKVSCKASGYTFISYTMHWVRQAPGQGLEWIGYINPRSGYTHYNQKLKDKATLTADKSASTAYMELSSLRSEDTAVYYCARSAYYDYDGFAYWGQGTLVTVSSGGGGSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 478 aa, Artificial sequence
  1st polypeptide  scFv-Fc fusion - CD3-tsm#4 (I30V/M48I/K67R) full length sequence
EVQLVQSGAEVKKPGASVKVSCKASGYTFVSYTMHWVRQAPGQGLEWIGYINPRSGYTHYNQKLKDRATLTADKSASTAYMELSSLRSEDTAVYYCARSAYYDYDGFAYWGQGTLVTVSSGGGGSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 75 - protein, 478 aa, Artificial sequence
  1st polypeptide  scFv-Fc fusion - CD3-tsm#5 (K67R)  full length sequence
EVQLVQSGAEVKKPGASVKVSCKASGYTFISYTMHWVRQAPGQGLEWMGYINPRSGYTHYNQKLKDRATLTADKSASTAYMELSSLRSEDTAVYYCARSAYYDYDGFAYWGQGTLVTVSSGGGGSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 76 - protein, 478 aa, Artificial sequence
  1st polypeptide  scFv-Fc fusion - CD3-tsm#6 ( (I30V)  full length sequence
EVQLVQSGAEVKKPGASVKVSCKASGYTFVSYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 77 - protein, 478 aa, Artificial sequence
  1st polypeptide  scFv-Fc fusion - CD3-tsm#7 (WT)  full length sequence
EVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGSGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 78 - protein, 448 aa, Artificial sequence
  2nd polypeptide  heavy chain - CD38A2- 3H10m1g1LALA-K full length sequence
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 448 aa, Artificial sequence
  2nd polypeptide - heavy chain - CD38A2g1LALA-K full length sequence
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREDWGGEFTDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 454 aa, Artificial sequence
  2nd polypeptide - heavy chain - CD38D8g1LALA-K full length sequence
QVQLVESGGGVVQPGGSLRLSCAASGFIVSTNYVHWVRQAPGKGLEWVSGIYSDPYTSYAYSDSVKGRFTISRDMSKNTVYLQMNRLRAEDTAVYYCARETNTGFSNSWYLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 454 aa, Artificial sequence
  2nd polypeptide - heavy chain - CD38D8- 3A1g1LALA-K full length sequence
QVQLVESGGGVVQPGGSLRLSCAASGFNVRTKYVHWVRQAPGKGLEWVSGIYSDPYTSYAYSDSVKGRFTISRDMSKNTVYLQMNRLRAEDTAVYYCARETNTGFSNSWYLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 454 aa, Artificial sequence
  2nd polypeptide - heavy chain - CD38D8- 3E1g1LALA-K full length sequence
QVQLVESGGGVVQPGGSLRLSCAASKLNVSPKYVHWVRQAPGKGLEWVSGIYSDPYTSYAYSDSVKGRFTISRDMSKNTVYLQMNRLRAEDTAVYYCARETNTGFSNSWYLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 454 aa, Artificial sequence
  2nd polypeptide - heavy chain - CD38D8- 1B5g1LALA-K full length sequence
QVQLVESGGGVVQPGGSLRLSCAASGLNARTNYVHWVRQAPGKGLEWVSGIYSDPYTSYAYSDSVKGRFTISRDMSKNTVYLQMNRLRAEDTAVYYCARETNTGFSNSWYLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 216 aa, Artificial sequence
  3rd polypeptide  light chain - CD38A2-3H10m1 LC full length sequence
QSALTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 85 - protein, 216 aa, Artificial sequence
  3rd polypeptide  light chain - CD38A2 LC full length sequence
QSALTQPPSASGTSGQRVTISCSGSSSNIGINFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 86 - protein, 216 aa, Artificial sequence
  3rd polypeptide  light chain - CD38D8 LC full length sequence
QPVLTQPPSASGTPGQRVTISCSGSSSNIGRNIVNWYQQLPGTTPKLLIYSNNQRPSGVPDRFSGSKSGTSASLAISGLHSEDEADYYCATWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 87 - protein, 497 aa, Artificial sequence
  BZ1 heterodimeric antibody 1st polypeptide
MEWSWVFLFFLSVTTGVHSQVQLVQSGAEVKKPGASVKVSCKASGYTFISYTMHWVRQAPGQGLEWIGYINPRSGYTHYNQKLKDRATLTADKSASTAYMELSSLRSEDTAVYYCARSAYYDYDGFAYWGQGTLVTVSSGGGGSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 88 - protein, 236 aa, Artificial sequence
  BZ1 heterodimeric antibody 3rd polypeptide
MSVPTQVLGLLLLWLTDARCQSVLTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 89 - protein, 467 aa, Artificial sequence
  BZ1 heterodimeric antibody 2nd polypeptide
MEWSWVFLFFLSVTTGVHSQVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 497 aa, Artificial sequence; identical to SEQ ID NO: 87
  BZ1S heterodimeric antibody 1st polypeptide

SEQ ID NO: 91 - protein, 236 aa, Artificial sequence; identical to SEQ ID NO: 88
  BZ1S heterodimeric antibody 3rd polypeptide

SEQ ID NO: 92 - protein, 467 aa, Artificial sequence
  BZ1S heterodimeric antibody 2nd polypeptide
MEWSWVFLFFLSVTTGVHSQVQLVESGGGLVKPGGSLRLSCAASGFTFSDDYMSWIRQAPGKGLEWVASVSSGRPTTYYADSVRGRFTISRDNAKNSLYLQMNSLRAEDTAVYYCAREGWSGEFTDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 94 - protein, 120 aa, Artificial sequence; identical to SEQ ID NO: 38
  1st polypeptide scFv g4-Fc-fusion  CD3 with dent - heavy chain variable sequence

SEQ ID NO: 95 - protein, 15 aa, Artificial sequence
  1st polypeptide scFv g4-Fc-fusion  CD3 with dent - linker sequence
GGGGSGGGGSGGGGS

SEQ ID NO: 96 - protein, 106 aa, Artificial sequence [sequence omitted]

SEQ ID NO: 97 - protein, 26 aa, Artificial sequence
  1st polypeptide scFv g4-Fc-fusion  CD3 with dent - hinge sequence
GGGGSSKYGPPCPPCPAPEFLGGPSV

SEQ ID NO: 98 - protein, 100 aa, Artificial sequence
  1st polypeptide scFv g4-Fc-fusion  CD3 with dent - CH2 sequence
FLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 99 - protein, 107 aa, Artificial sequence
  1st polypeptide scFv g4-Fc-fusion  CD3 with dent - CH3 sequence (with dent)
GQPREPQVYTLPPSQEEMTKNQVSLTCLVKGFYPSDIAVEWESNGQPENNYKTTPPVLDSDGSFLLYSRLTVDKSRWQEGNVFSCSVMHEALHNHYTQKSLSLSLGK

SEQ ID NO: 100 - protein, 474 aa, Artificial sequence
  1st polypeptide scFv g4-Fc-fusion  CD3 with dent - full-length sequence
EVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSRLTVDKSRWQEGNVFSCSVMHEALHNHYTQKSLSLSLGK

SEQ ID NO: 101 - protein, 124 aa, Artificial sequence; identical to SEQ ID NO: 8
  2nd polypeptide IgG4 heavy chain  CD38D8- 1B5g1LALA-K (with bump) heavy chain variable sequence

SEQ ID NO: 102 - protein, 103 aa, Artificial sequence
  2nd polypeptide IgG4 heavy chain  CD38D8- 1B5g1LALA-K (with bump) heavy chain constant sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYG

SEQ ID NO: 103 - protein, 17 aa, Artificial sequence
  2nd polypeptide IgG4 heavy chain  CD38D8- 1B5g1LALA-K (with bump) hinge sequence
PPCPPCPAPEFLGGPSV

SEQ ID NO: 104 - protein, 100 aa, Artificial sequence; identical to SEQ ID NO: 98
  2nd polypeptide IgG4 heavy chain  CD38D8- 1B5g1LALA-K (with bump) CH2 sequence

SEQ ID NO: 105 - protein, 107 aa, Artificial sequence
  2nd polypeptide IgG4 heavy chain  CD38D8- 1B5g1LALA-K (with bump) CH3 sequence with bump
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 451 aa, Artificial sequence
  2nd polypeptide IgG4 heavy chain  CD38D8- 1B5g1LALA-K (with bump) full length sequence
QVQLVESGGGVVQPGGSLRLSCAASGFIVSTNYVHWVRQAPGKGLEWVSGIYSDPYTSYAYSDSVKGRFTISRDMSKNTVYLQMNRLRAEDTAVYYCARETNTGFSNSWYLDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 301 aa, Macaca fascicularis
MANCEFSPVSGDKPCCRLSRRAQVCLGVCLLVLLILVVVVAVVLPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSGI

SEQ ID NO: 108 - protein, 304 aa, Mus musculus
MANYEFSQVSGDRPGCRLSRKAQIGLGVGLLVLIALVVGIVVILLRPRSLLVWTGEPTTKHFSDIFLGRCLIYTQILRPEMRDQNCQEILSTFKGAFVSKNPCNITREDYAPLVKLVTQTIPCNKTLFWSKSKHLAHQYTWIQGKMFTLEDTLLGYIADDLRWCGDPSTSDMNYVSCPHWSENCPNNPITVFWKVISQKFAEDACGVVQVMLNGSLREPFYKNSTFGSVEVFSLDPNKVHKLQAWVMHDIEGASSNACSSSSLNELKMIVQKRNMIFACVDNYRPARFLQCVKNPEHPSCRLNT

SEQ ID NO: 109 - protein, 303 aa, Rattus norvegicus
MANYEFSQVSEDRPGCRLTRKAQIGLGVGLLLLVALVVVVVIVLWPRSPLVWKGKPTTKHFADIILGRCLIYTQILRPEMRDQDCKKILSTFKRGFISKNPCNITNEDYAPLVKLVTQTIPCNKTLFWSKSKHLAHQYTWIQGKMFTLEDTLLGYIADDLRWCGDPSTSDMNYDSCPHWSENCPNNPVAVFWNVISQKFAEDACGVVQVMLNGSLSEPFYRNSTFGSVEVFNLDPNKVHKLQAWVMHDIKGTSSNACSSPSINELKSIVNKRNMIFACQDNYRPVRFLQCVKNPEHPSCRLNV

SEQ ID NO: 110 - protein, 5 aa, Artificial sequence
  Hinge linker sequence
GGSGG

SEQ ID NO: 111 - protein, 12 aa, Artificial Sequence
  Synthetic peptide linker
  misc_feature (3)..(12): “Ser Gly” may or may not be present
SGSGSGSGSGSG

SEQ ID NO: 112 - protein, 18 aa, Artificial Sequence
  Synthetic peptide linker
  misc_feature (4)..(18): “Ser Gly Gly” may or may not be present
SGGSGGSGGSGGSGGSGG

SEQ ID NO: 113 - protein, 24 aa, Artificial Sequence
  Synthetic peptide linker
  misc_feature (5)..(24): “Ser Gly Gly Gly” may or may not be present
SGGGSGGGSGGGSGGGSGGGSGGG

SEQ ID NO: 114 - protein, 18 aa, Artificial Sequence
  Synthetic peptide linker
  misc_feature (4)..(18): “Ser Ser Gly” may or may not be present
SSGSSGSSGSSGSSGSSG

SEQ ID NO: 115 - protein, 12 aa, Artificial Sequence
  Synthetic peptide linker
  misc_feature (3)..(12): “Gly Ser” may or may not be present
GSGSGSGSGSGS

SEQ ID NO: 116 - protein, 18 aa, Artificial Sequence
  Synthetic peptide linker
  misc_feature (4)..(18): “Gly Gly Gly” may or may not be present
GGGGGGGGGGGGGGGGGG

SEQ ID NO: 117 - protein, 30 aa, Artificial Sequence
  Synthetic peptide linker
  misc_feature (6)..(30): “Gly Ser Gly Gly Ser” may or may not be present
GSGGSGSGGSGSGGSGSGGSGSGGSGSGGS

SEQ ID NO: 118 - protein, 18 aa, Artificial Sequence
  Synthetic peptide linker
  misc_feature (4)..(18): “Gly Ser Gly” may or may not be present
GSGGSGGSGGSGGSGGSG

SEQ ID NO: 119 - protein, 30 aa, Artificial Sequence
  Synthetic peptide linker
  misc_feature (6)..(30): “Gly Gly Gly Gly Ser” may or may not be present
GGGGSGGGGSGGGGSGGGGSGGGGSGGGGS

SEQ ID NO: 120 - protein, 24 aa, Artificial Sequence
  Synthetic peptide linker
  misc_feature (5)..(24): “Gly Gly Gly Ser” may or may not be present
GGGSGGGSGGGSGGGSGGGSGGGS

SEQ ID NO: 121 - protein, 42 aa, Artificial Sequence
  Synthetic peptide linker
  misc_feature (8)..(42): “Gly Gly Gly Gly Ser Gly Ser” may or may not be present
GGGGSGSGGGGSGSGGGGSGSGGGGSGSGGGGSGSGGGGSGS

SEQ ID NO: 122 - protein, 48 aa, Artificial Sequence
  Synthetic peptide linker
  misc_feature (9)..(48): “Gly Gly Gly Gly Ser Gly Gly Ser” may or may not be present
GGGGSGGSGGGGSGGSGGGGSGGSGGGGSGGSGGGGSGGSGGGGSGGS

SEQ ID NO: 123 - protein, 18 aa, Artificial Sequence
  Synthetic peptide linker
  misc_feature (4)..(18): “Gly Gly Ser” may or may not be present
GGSGGSGGSGGSGGSGGS

SEQ ID NO: 124 - protein, 11 aa, Artificial sequence
  Synthetic peptide linker
TSGSGGSGGSV

SEQ ID NO: 125 - protein, 19 aa, Artificial sequence
  Mouse Ig gamma leader peptide sequence
MEWSWVFLFFLSVTTGVHS

SEQ ID NO: 126 - protein, 20 aa, Artificial sequence
  Exemplary signal peptide
MSVPTQVLGLLLLWLTDARC

SEQ ID NO: 127 - protein, 10 aa, Artificial sequence
  Exemplary hinge region sequence
EPKSCDKTHT

SEQ ID NO: 128 - protein, 4 aa, Artificial sequence
  Exemplary hinge region sequence
  misc_feature (3)..(3): Xaa can be Pro, Arg, or Ser
CPXC

SEQ ID NO: 129 - protein, 9 aa, Artificial sequence
  Exemplary hinge region sequence
PAPELLGGP

SEQ ID NO: 130 - protein, 12 aa, Artificial sequence
  Exemplary hinge region sequence
SVFLFPPKPKDT

SEQ ID NO: 131 - protein, 23 aa, Artificial sequence
  Exemplary hinge region sequence
EPKSCDKTHTCPPCPAPELLGGP